(19) United States
(12) Patent Application Publication
(10) Pub. No.: US 2002/0057050 A1
(43) Pub. Date: May 16, 2002

(54) Organic light emitting diode devices using aromatic amine compounds with high and tunable glass transition temperatures

(76) Inventor: Xiaobo Shi, Poughquag, NY (US)

Correspondence Address: PATENT DEPARTMENT, SKADDEN, ARPS, SLATE, MEAGHER & FLOM LLP, FOUR TIMES SQUARE, NEW YORK, NY, 10036, US

(21) Appl. No.: 09/894,744

(22) Filed: Jun. 28, 2001

Related U.S. Application Data
(60) Provisional application No. 60/214,796, filed on Jun. 28, 2000.

(51) Int. Cl. G03G 15/02; H01J 1/62; H01J 63/04; C07D 209/82; C07D 223/18

(52) U.S. Cl. 313/504; 430/58.15; 430/58.6; 430/58.5; 430/58.65; 540/588; 548/440; 549/74; 564/433

(57) ABSTRACT

The present invention relates to a novel class of thermostable hole-injection and hole-transport compounds having tunable glass transition temperatures and ionization potentials for use in organic light emitting diode (“OLED”) devices. In particular, the compounds of the present invention comprise a trityl aniline core structure with various substituents attached to the nitrogen group, the structures of which allow for the adjustment of the glass transition temperatures and ionization potentials of the compounds. The present invention also relates to microdisplay devices comprising the compounds of the present invention in the hole-injection/hole-transport layers.

### CROSS-REFERENCE TO RELATED APPLICATIONS

This application claims priority to United States Provisional Patent Application No. 60/214,796, filed Jun. 28, 2000.

### STATEMENT REGARDING FEDERALLY SPONSORED RESEARCH OR DEVELOPMENT

This research was sponsored by U.S. Government DUAP contract number F33615-98-2-5156.

### BACKGROUND OF THE INVENTION

Organic electroluminescence (EL) in the form of organic light emitting diode (“OLED”) devices, a new generation of high-resolution display technology resulting from optoelectronic science, is moving from a simple curiosity in the laboratory to the reality of commercial use. OLED devices comprise an anode, a cathode and an electroluminescent medium made up of extremely thin layers (typically less than 1.0 micrometer in combined thickness) separating the anode and the cathode. A basic two-layer light emitting diode comprises one organic layer that is specifically chosen to inject and transport holes and a second organic layer that is specifically chosen to inject and transport electrons. The interface between the two layers provides an efficient site for the recombination of the injected hole-electron pair, which results in electroluminescence. The electroluminescent medium can comprise additional layers, including, but not limited to, an emitter layer between the hole- injection and transport and the electron-injection and transport layers in which recombination of holes and electrons occurs. Since light emission is directly related to current density through the organic electroluminescent medium, the thin layers coupled with increased charge injection and transport efficiencies have allowed acceptable light emission levels (e.g., brightness levels capable of being visually detected in ambient light) to be achieved with low applied voltages in ranges compatible with integrated circuit drivers, such as field effect transistors.

OLED devices made by vacuum sublimation exhibit the best performance. Lifetimes in the range of 5,000 to 30,000 hours at a starting level of brightness of several hundred cd/m² have been reported for room temperature operations at relatively low current density. A high luminance display at relatively high temperatures, eg., between 100° C. and 150° C., consumes a non-negligible amount of power, which will in turn generate a significant amount of heat that will affect the storage and operation of the optoelectronic devices. In addition, many processing steps, such as direct patterning of color filters directly on top of the OLED devices and sealing of the devices, are performed at elevated temperatures (for example, above 130° C.). Under such operating and processing conditions, the excessive heat generated can accelerate the degradation of the optoelectronic devices due to the low thermal tolerance of organic molecular solids comprising the OLED.

Analysis of the thermal behavior of OLED devices indicates that hole-transport and hole-injection compounds have tended to be an unstable part of the electroluminescent medium of OLEDs and may prevent the devices from being operated at relatively high temperatures. These materials are thought to undergo a morphological change when exposed to elevated temperatures or when stored for long periods of time. Since efficient operation of the hole-injection and hole-transport layers depends on their amorphous nature, morphological changes may lead to degradation in performance of the OLED. The temperature at which morphological changes occur and an amorphous material becomes crystalline is the glass transition temperature (“T_{g}”) of the material and is closely correlated with OLED device degradation.

The glass transition temperatures of hole-injection and hole-transport compounds have generally been below 100° C. At temperatures greater than 100° C., conventional hole-transport materials, such as NPB and TPD, begin to undergo a phase transition from amorphous to polycrystalline, which significantly reduces hole mobility and electroluminescence quantum yield, and ultimately leads to device failure. Of the known materials used to fabricate OLED devices, hole-transport materials have the lowest T_{g} values. For example, the T_{g} of _{α}-NPB is 96° C., compared to 175° C. for the electron-transport material, Alq₃. Therefore, new molecular design strategies for the preparation of hole-injection and hole-transport materials that are thermally and electrochemically stable and that have high glass transition temperatures are critical for production of OLED devices with high brightness and long lifetime.

In the development of novel hole-injection and hole-transport materials, it is necessary to consider several factors, which include, but are not limited to, the following. First, the energy levels of the highest occupied molecular orbital and the lowest unoccupied molecular orbital of the material should closely match those of the materials in the adjacent layers. Second, as discussed above, the materials should possess high glass transition temperatures and thermal stability. Third, the hole-injection and hole-transport materials should not undergo a morphology change at a temperature lower than their T_{g} values. Fourth, the materials should have good hole mobility at the interfaces with adjacent layers. Fifth, the materials should be robust in order to minimize the morphology change due to recrystallization or rearrangement during the storage and operation of OLED display devices. Sixth, the materials should be easily fabricated into OLED and other optoelectronic active devices (ideally by vapor deposition) to form amorphous and uniform films.

Tertiary aromatic amines have been widely used as hole-injection and hole-transport materials in OLED display devices. Some tertiary aromatic amines have been found to possess one or more of the aforementioned characteristics and to function adequately as hole-injection and hole-transport materials due to their suitable ionization potentials and good hole mobility. In general, tertiary aromatic amines are fairly robust and are somewhat thermally, photochemically, and electrochemically stable. The structures of some commonly-used hole-injection and hole-transport materials are shown in Table I below, along with their T_{g}s and ionization potentials (“IP”).

TABLE I

| Material | T_{g} (° C.) | IP (eV) |
|---|---|---|
| 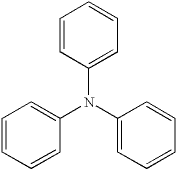 | 43 | ˜4.80 |
| 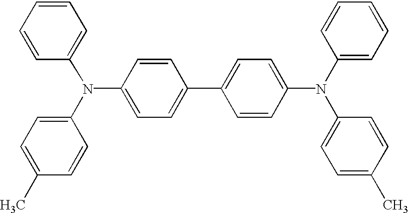 | 63 | ˜5.15 |
| 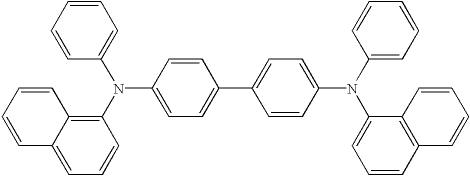 | 95 | ˜5.20 |
| [chemical structure] | 150 | — |
| 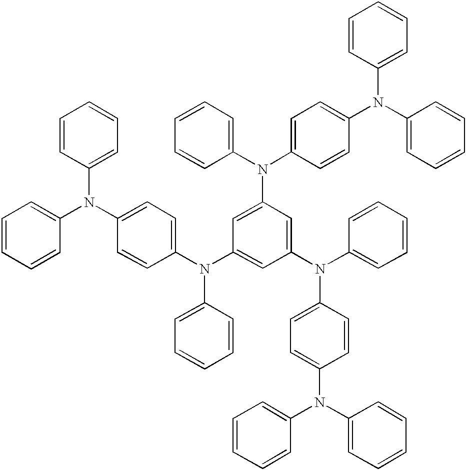 | 108 | ˜5.30 |
| 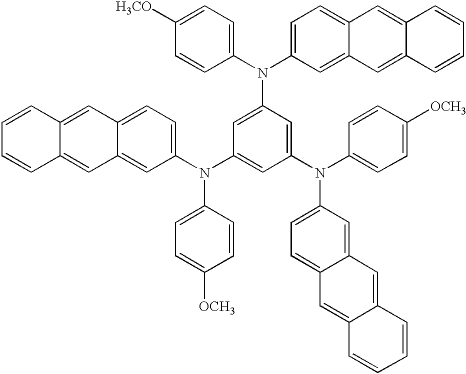 | 123 | ˜5.16 |
| 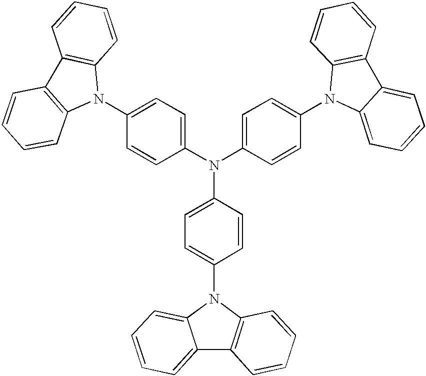 | 150 | ˜5.70 |
| 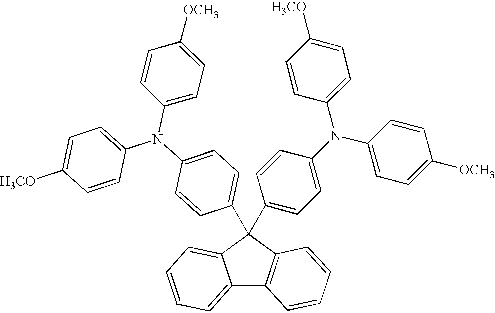 | 103 | ˜5.42 |
| 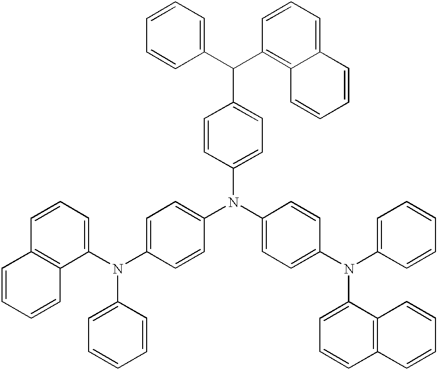 | 113 | ˜5.10 |
| 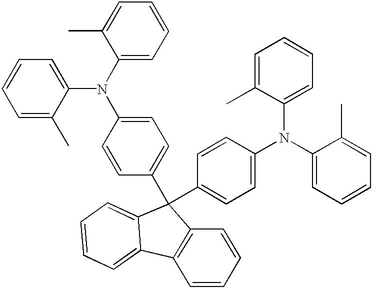 | 113 | ˜5.80 |

The simplest aromatic amine, triphenyl amine (“TPA”), has been used as a hole-transport material in OLEDs. However, simple aromatic amines do not form stable amorphous films. TPD, with a T_{g} of 63° C., easily undergoes crystallization during operation and storage in an inert atmosphere of an OLED device comprising the compound, as evidenced by X-ray diffraction and scanning electron microscopy. A substitution of the methyl phenyl group of TPD with a naphthyl group produces α-NPB, which has a reported T_{g} value as high as 96° C. Devices fabricated using α-NPB as hole-injection and/or hole-transport materials perform better and are more thermostable than devices fabricated using TPD. However, hole-injection and/or hole-transport materials with a T_{g} value of less than 100° C. do not permit OLED devices to be operated and stored at temperatures above 100° C. Higher storage, processing and operating temperatures of OLED display devices demand the development of new organic hole-injection and hole-transport materials with higher T_{g} values and better thermal stability.

In order to meet these needs, a number of organic aromatic amine materials with higher T_{g} values have been developed. These include linear oligomers and “starburst” tertiary aromatic amine compounds. The structures of a few illustrative compounds are shown in Table I above and include: “starburst” molecules ρ-DPA-TDAB (T_{g}=108° C.), and 1-TNATA (T_{g}=113° C.); “starburst” molecules TDAB-8 (T_{g}=123° C.) and TCTA (T_{g}=150° C.), which comprise rigid peripheral anthrecenyl and carbazolyl groups, respectively; and the linear oligomer TPPE, which has a higher T_{g} (150° C.) than many “starburst” molecules. Devices that incorporate these molecules in the hole-injection and hole-transport layers exhibit improved thermal stability when compared to devices that incorporate TPD in these layers.

A third family of molecules that can be used as hole-injection and hole-transport materials in OLEDs includes fluorene-based molecules (examples include ρ-methoxy FTPD and o-methyl FTPD). As reported by Okutsu et al. ((1997) IEEE Trans. Electron. Devices 44:1302-06), subtle changes in the molecular structure, such as altering the chemical nature of the substituents on the periphery of the molecule, can cause dramatic variation in T_{g} and ionization potential. By varying the chemical nature and positions of the substituents, molecules with T_{g} values ranging from 80° C. to 118° C. and ionization potentials ranging from 5.40eV to 5.80eV were prepared and evaluated. However, reported T_{g} values were still relatively low.

Another family of hole transport materials comprises spiro-bifluorene-based aromatic amine compounds. Spiro-bifluorene is used as the core structure, and various aromatic amines are attached to it at one or more of the 2-, 2′-, 9- and 9′-positions to afford hole transport materials, such as SBF-TPD (T_{g}=133° C.) and SBF-NPB (T_{g}=147° C.), with higher T_{g} values and improved thermal stability (structures not shown in Table I).

Discussion or citation of a reference herein shall not be construed as an admission that such reference is prior art to the present invention.

### BRIEF SUMMARY OF THE INVENTION

In a first embodiment, the present invention relates to a hole-injection or hole-transport compound of formula 1:

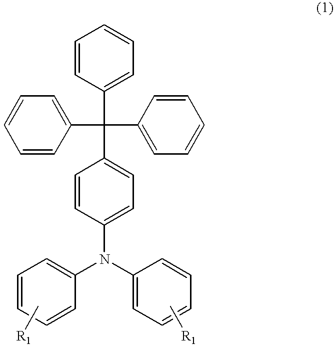

wherein each R₁ is independently selected from the group consisting of hydrogen, C₁-C₆ straight or branched chain alkyl, alkoxy, —CN, —Cl, —F, —CF₃, —SR, —SiR, vinyl that is unsubstituted or substituted at terminal carbon position(s) with C₁-C₆ alkyl or an aromatic group, aryl that is unsubstituted or substituted at para-, meta- or ortho- positions with a C₁-C₆ alkyl, C₁-C₈ alkoxy or —SR,

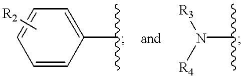

R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C₈ alkoxy or —SR, and

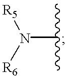

R₃, R₄, R₅ and R₆ are each independently selected from the group consisting of:

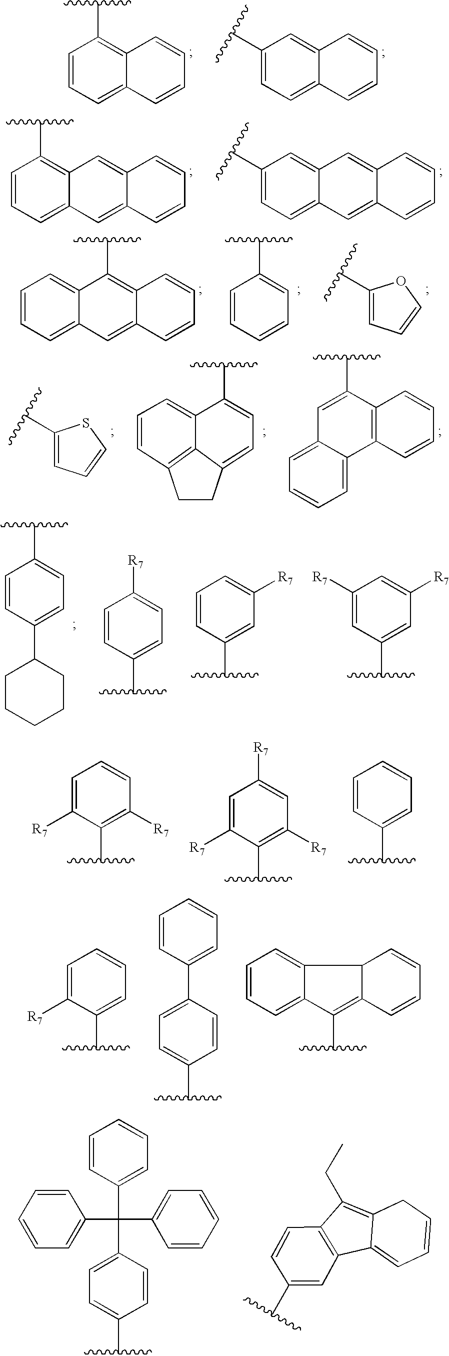

or R₃ and R₄ taken together with the nitrogen atom to which they are attached or R₅ and R₆ taken together with the nitrogen atom to which they are attached are independently selected from the group consisting of:

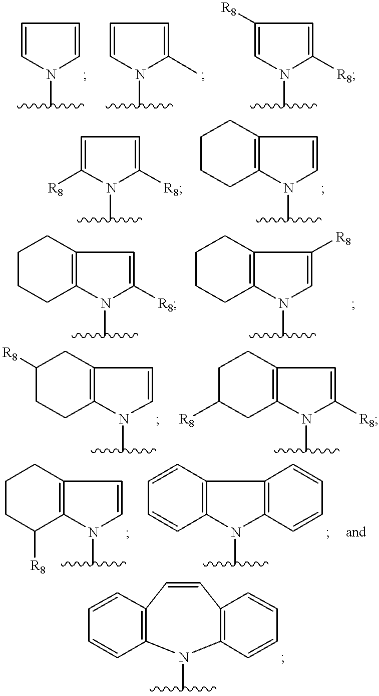

R₇ and R₈ are each independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

In a second embodiment, the present invention relates to a hole-injection or hole-transport compound of formula 2:

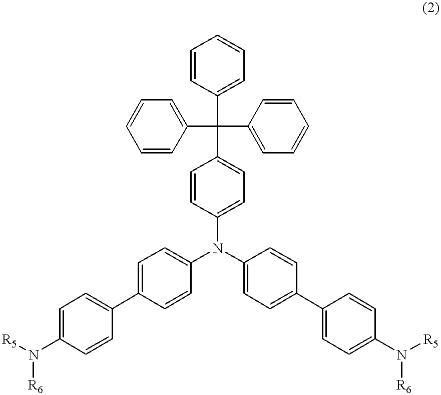

wherein R₅ and R₆ are each independently selected from the group consisting of

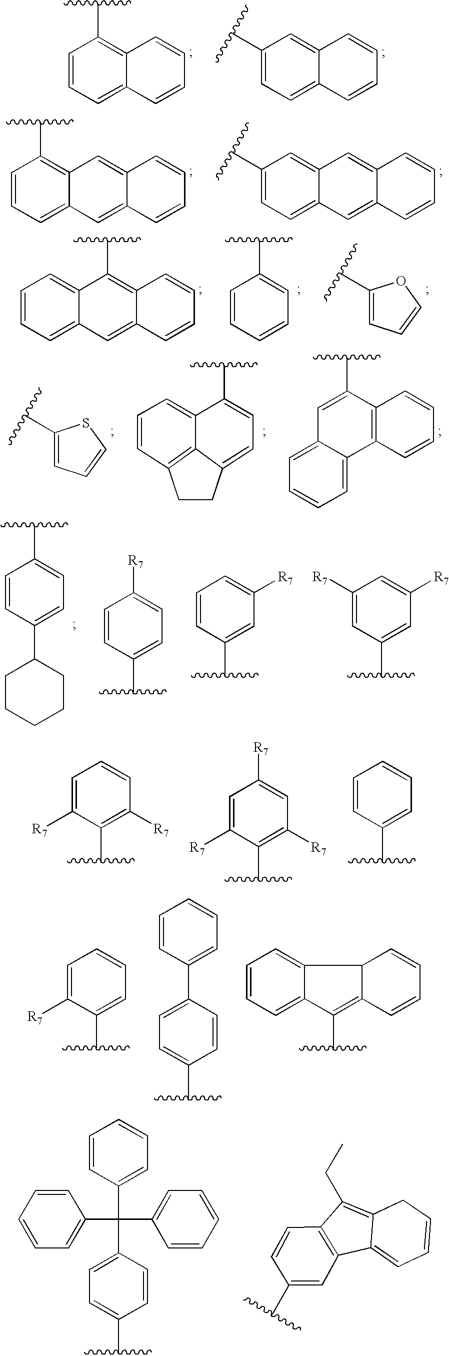

or R₅ and R₆ taken together with the nitrogen atom to which they are attached are independently selected from the group consisting of:

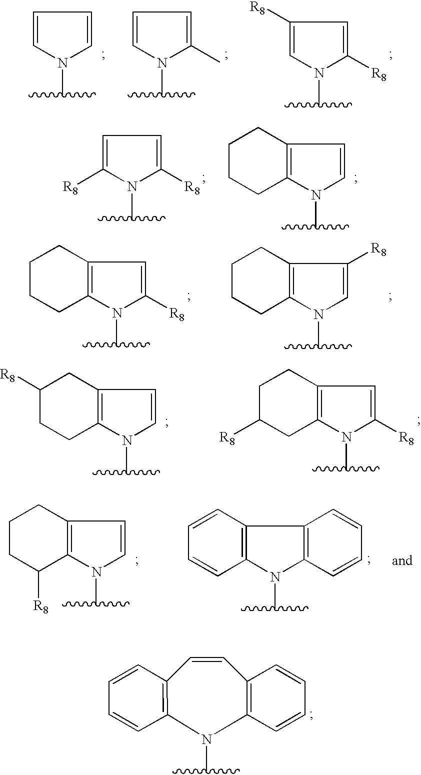

R₇ and R₈ are each independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

In a third embodiment, the present invention relates to a hole-injection or hole-transport compound of formula 3:

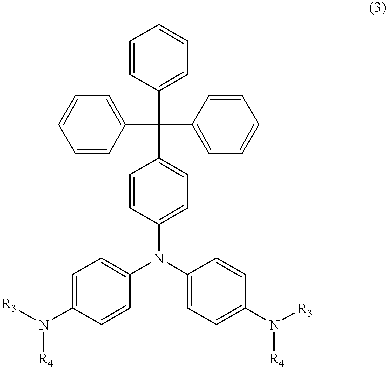

wherein R₃ and R4 are each independently selected from the group consisting of:

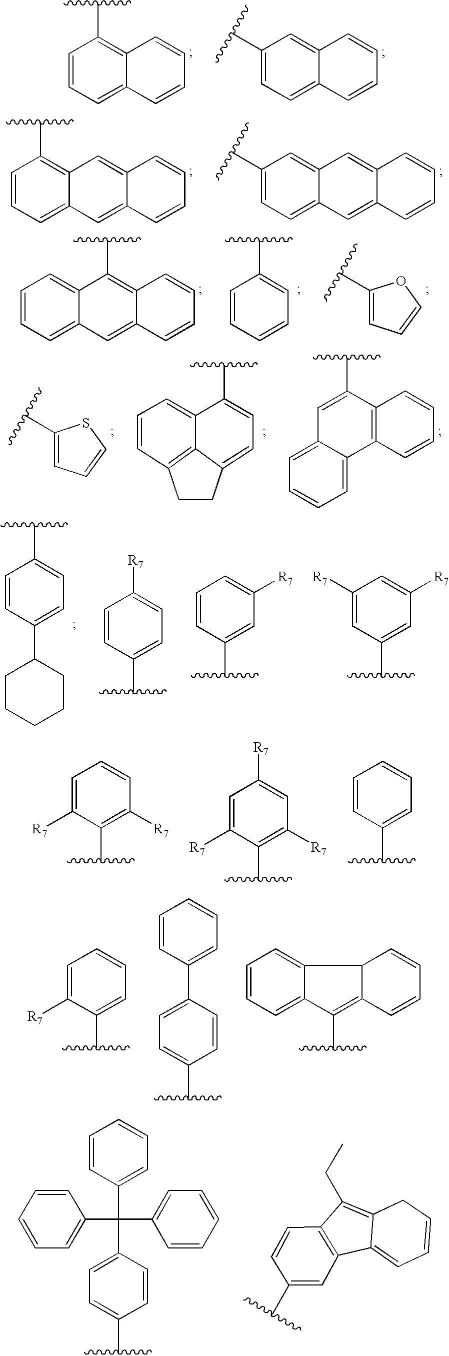

or R₃ and R₄ taken together with the nitrogen atom to which they are attached are independently selected from the group consisting of:

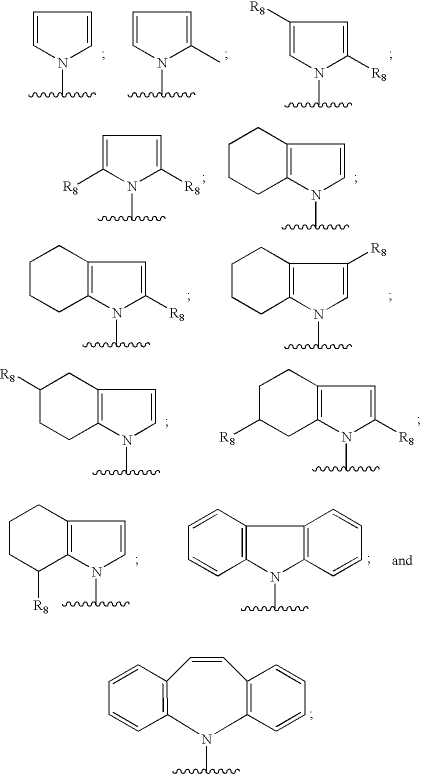

R₇ and R₈ are each independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

In a fourth embodiment, the present invention relates to a hole-injection or hole-transport compound of formula 4:

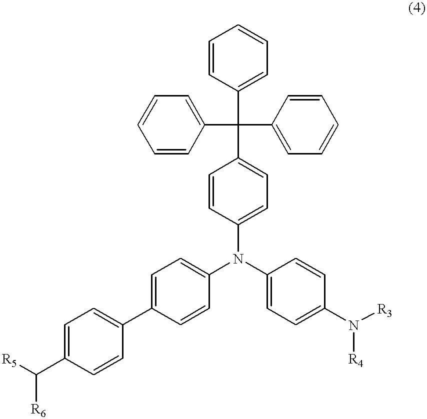

wherein R₃, R₄, R₅ and R₆ are each independently selected from the group consisting of

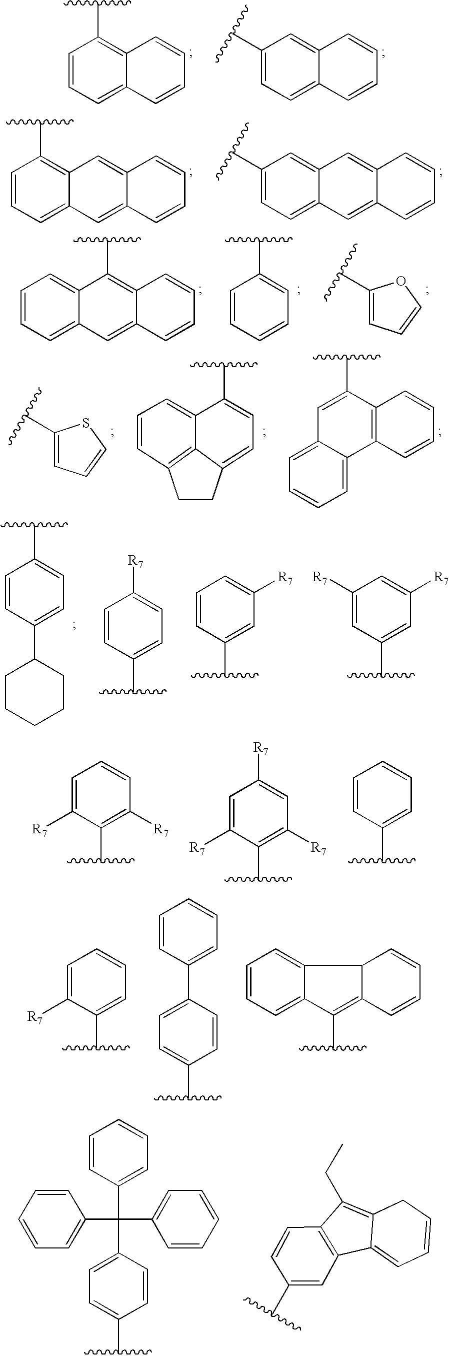

or R₃ and R₄ taken together with the nitrogen to which they are attached or R₅ and R₆ taken together with the nitrogen atom to which they are attached are selected from the group consisting of:

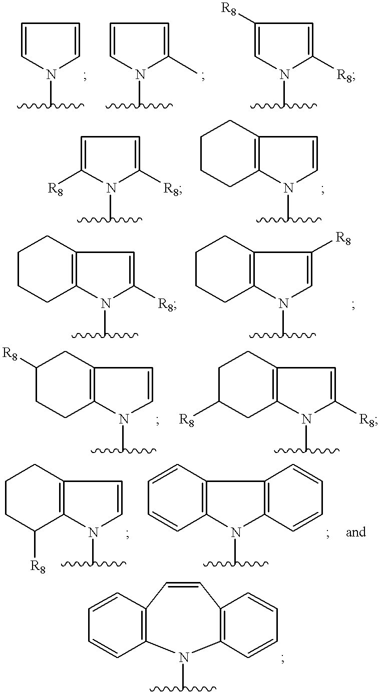

R₇ and R₈ are each independently selected from the group consisting of —OR₉, C₁-C₄ alkyl, aryl, —SCH₃, —CF₃, —Cl, —Br, —NO₂, —SR, —SiR and —OOR₉;

R is C₁-C₆ straight or branched chain alkyl; and

R₉ is selected from the group consisting of C₁-C₆ alkyl and aryl.

In a fifth embodiment, the present invention relates to an organic light emitting diode device comprising: (a) a cathode; (b) an anode; and (c) at least two organic layers between the anode and the cathode, wherein the at least two organic layers comprise a first organic layer formed from at least one electron-injection/electron-transport material and a second organic layer formed from at least one hole-injection/hole-transport material, wherein the electron-injection/electron-transport material is adjacent to the cathode and the hole-injection/hole-transport material is adjacent to the anode, the at least one hole-injection/hole-transport material comprising a compound of formula 1, wherein the substituent groups are as recited above.

In a sixth embodiment, the present invention relates to An organic light emitting diode device comprising: (a) a cathode; (b) an anode; (c) a layer formed from at least one electron-injection/electron-transport material that is adjacent to the cathode; (d) a hole-injection layer that is adjacent to the anode; and (e) at least one hole-transport layer that is adjacent to the hole-injection layer, wherein at least one of the hole-injection and hole-transport layers comprises a compound of formula 1, wherein the substituents are as recited above.

In a seventh embodiment, the present invention relates to a microdisplay device, comprising: (a) at least one bottom electrode that is an anode; (b) at least one top electrode that is a cathode; and (c) at least two organic layers between the at least one bottom electrode and the at least one top electrode, wherein the at least two organic layers comprise a first organic layer formed from at least one electron-injection/electron-transport material that is adjacent to the at least one cathode and a second organic layer formed from at least one hole-injection/hole-transport material that is adjacent to the at least one anode, the at least one hole-injection/hole-transport material comprising a compound of formula 1, wherein the substituents are as recited above.

In an eighth embodiment, the present invention relates to an organic light emitting diode device comprising: (a) a cathode; (b) an anode; and (c) at least two organic layers between the anode and the cathode, wherein the at least two organic layers comprise a first organic layer formed from at least one electron-injection/electron-transport material and a second organic layer formed from at least one hole-injection/hole-transport material, wherein the electron-injection/electron-transport material is adjacent to the cathode and the hole-injection/hole-transport material is adjacent to the anode, the at least one hole-injection/hole-transport material comprising a compound of formula 2, wherein the substituents are as recited above.

In ninth embodiment, the present invention relates to an organic light emitting diode device comprising: (a) a cathode; (b) an anode; and (c) at least two organic layers between the anode and the cathode, wherein the at least two organic layers comprise a first organic layer formed from at least one electron-injection/electron-transport material and a second organic layer formed from at least one hole-injection/hole-transport material, wherein the electron-injection/electron-transport material is adjacent to the cathode and the hole-injection/hole-transport material is adjacent to the anode, the at least one hole-injection/hole-transport material comprising a compound of formula 3, wherein the substituents are as recited above.

In a tenth embodiment, the present invention relates to an organic light emitting diode device comprising: (a) a cathode; (b) an anode; and (c) at least two organic layers between the anode and the cathode, wherein the at least two organic layers comprise a first organic layer formed from at least one electron-injection/electron-transport material and a second organic layer formed from at least one hole-injection/hole-transport material, wherein the electron-injection/electron-transport material is adjacent to the cathode and the hole-injection/hole-transport material is adjacent to the anode, the at least one hole-injection/hole-transport material comprising a compound of formula 4, wherein the substituents are as recited above.

In an eleventh embodiment, the present invention relates to a hole-injection or hole-transport compound of formula 1, wherein

R₃, R₄, R₅ and R₆ are each independently selected from the group consisting of:

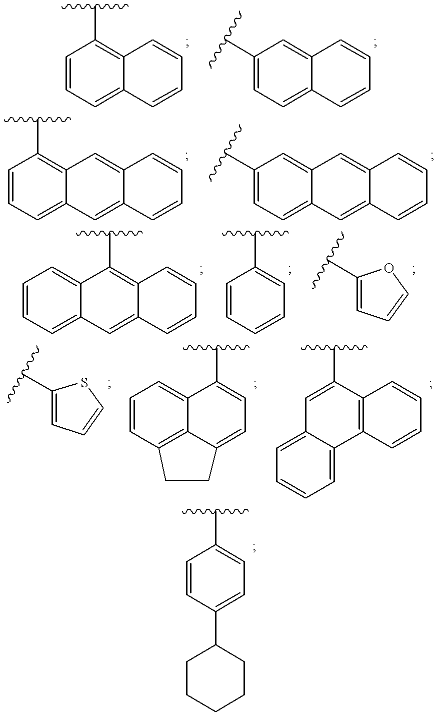

or R₃ and R₄ taken together with the nitrogen atom to which they are attached or R₅ and R₆ taken together with the nitrogen atom to which they are attached are independently selected from the group consisting of:

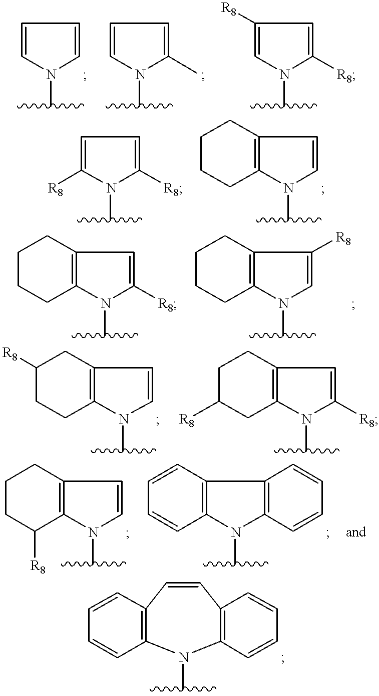

and all other substituents are as above.

In a twelfth embodiment, the present invention relates to a hole-injection or hole-transport compound of formula 2:

wherein R₅ and R₆ are each independently selected from the group consisting of

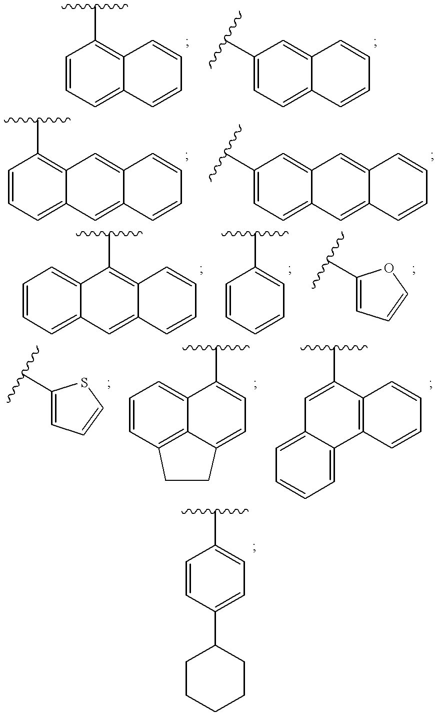

or R₅ and R₆ taken together with the nitrogen atom to which they are attached are independently selected from the group consisting of:

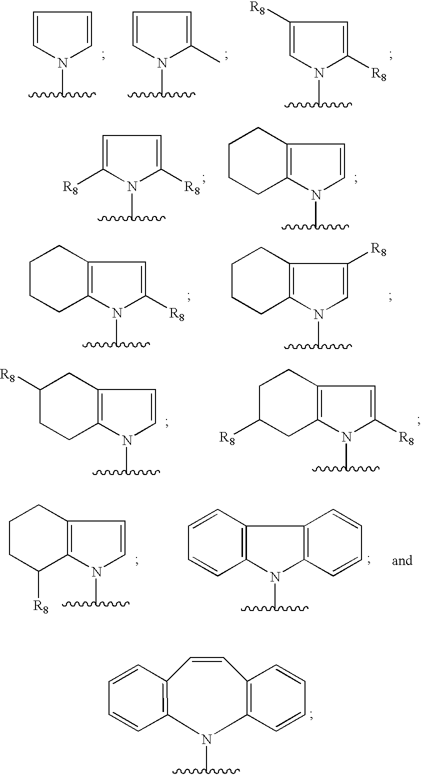

and all other substituents are as above.

In a thirteenth embodiment, the present invention relates to a hole-injection or hole-transport compound of formula 3:

wherein R₃ and R₄ are each independently selected from the group consisting of:

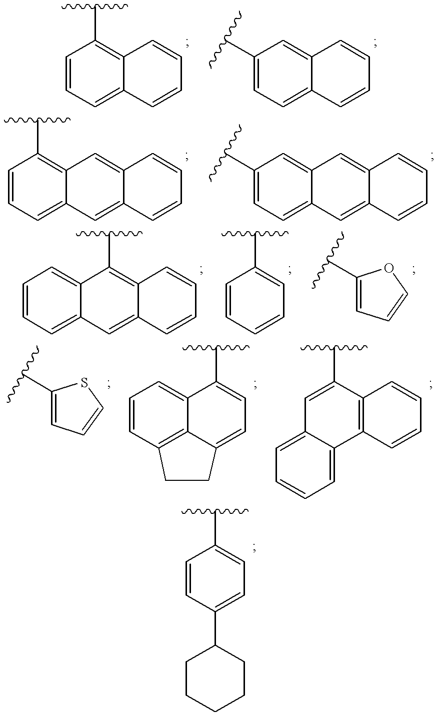

or R₃ and R₄ taken together with the nitrogen atom to which they are attached are independently selected from the group consisting of:

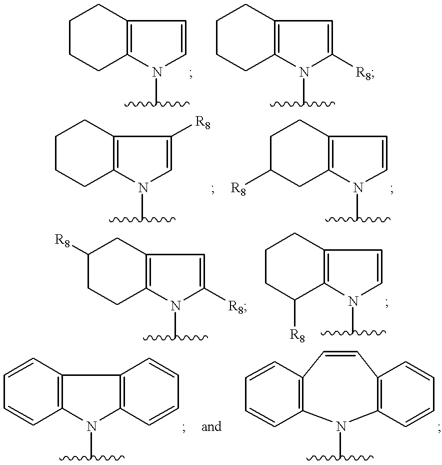

and all other substituents are as above.

In a fourteenth embodiment, the present invention relates to a hole-injection or hole-transport compound of formula 4:

wherein R₃, R_{4,} R₅ and R₆ are each independently selected from the group consisting of

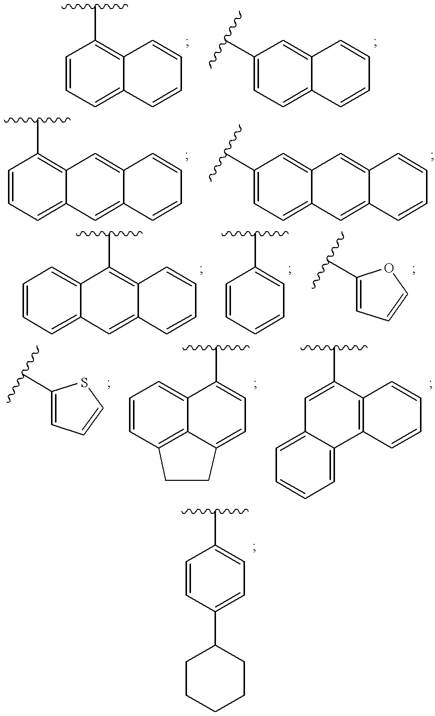

or R₃ and R₄ taken together with the nitrogen to which they are attached or R₅ and R₆ taken together with the nitrogen atom to which they are attached are selected from the group consisting of:

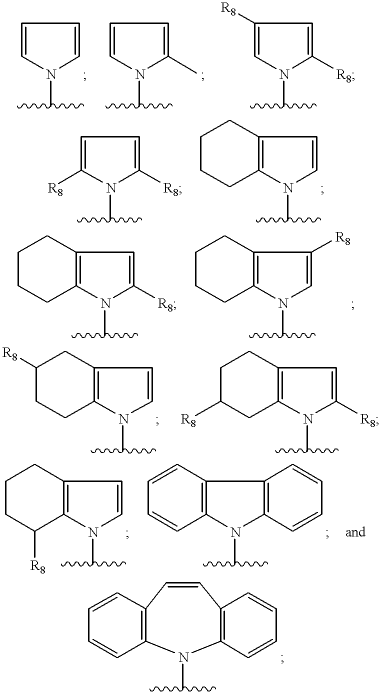

and all other substituents are as above.

### BRIEF DESCRIPTION OF THE SEVERAL VIEWS OF THE DRAWING

FIG. 1 shows an OLED stack according to the present invention.

FIG. 2 shows an OLED stack comprising a bottom anode and a top cathode on a substrate.

FIG. 3 shows an OLED stack comprising a bottom cathode and a top anode on a substrate.

FIG. 4 shows a preferred OLED stack.

### DETAILED DESCRIPTION OF THE INVENTION

OLEDs can be fabricated by any method known to those skilled in the art. In one embodiment, OLEDs are formed by vapor deposition of each layer. In a preferred embodiment, OLEDs are formed by thermal vacuum vapor deposition.

“Bottom electrode,” as used herein, means an electrode that is deposited directly onto the substrate.

“Top electrode,” as used herein, means an electrode that is deposited at the end of the OLED that is distal to the substrate.

“Hole-injection layer,” as used herein, is a layer into which holes are injected from an anode when a voltage is applied across an OLED.

“Hole-transport layer,” as used herein, is a layer having high hole mobility and high affinity for holes that is between the anode and the emitter layer. It will be evident to those of skill in the art that the hole-injection layer and the hole-transport layer can be a single layer, or they can be distinct layers comprising different chemical compounds. A compound of formula I is useful both in both hole-injection and hole-transport layers.

“Electron-injection layer,” as used herein, is a layer into which electrons are injected from a cathode when a voltage is applied across an OLED.

“Electron-transport layer,” as used herein, is a layer having high electron mobility and high affinity for electrons that is between the cathode and the emitter layer. It will be evident to those of skill in the art that the electron-injection layer and the electron-transport layer can be a single layer, or they can be distinct layers comprising different chemical compounds.

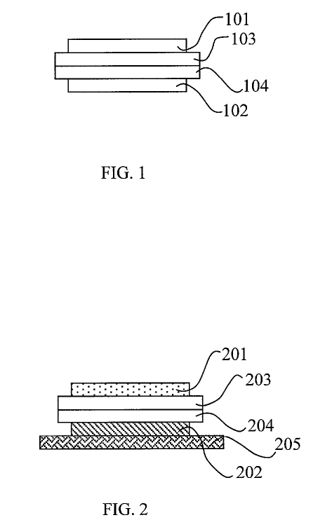

In one embodiment, shown in FIG. 1, an OLED comprises a bottom electrode 102, which is either an anode or a cathode, a top electrode 101, which is a cathode if the bottom electrode is an anode and which is an anode if the bottom electrode is a cathode, and an electroluminescent medium having at least two layers 103, 104, one comprising at least one hole-injection/hole-transport material that is adjacent to the anode and the other comprising at least one electron-injection/electron-transport layer that is adjacent to the cathode.

In another embodiment shown in FIG. 2, the top electrode is the cathode 201 and the bottom electrode, which is deposited directly onto the substrate 205, is the anode 202. Between the cathode and the anode are an electron-injection/electron-transport layer 203 adjacent to the cathode 201 and a hole-injection/hole-transport layer 204 adjacent to the anode 202.

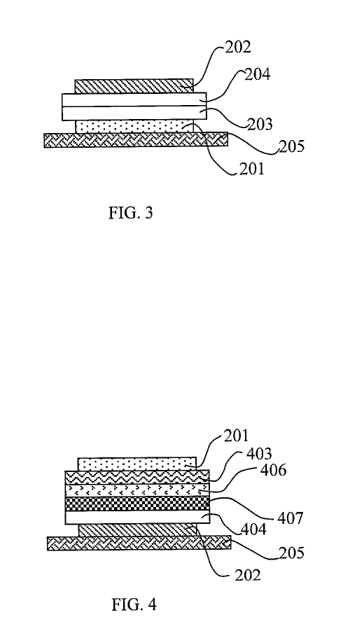

In another embodiment shown in FIG. 3, the top electrode is the anode 202 and the bottom electrode, which is deposited directly onto the substrate 205, is the cathode 201. Between the cathode and the anode are a hole-injection/hole-transport layer 204 adjacent to the anode 202 and an electron-injection/electron-transport layer 203 adjacent to the cathode 201.

In yet another embodiment shown in FIG. 4, the top electrode is the cathode 201 and the bottom electrode, which is deposited directly onto the substrate 205, is the anode 202. The OLED further comprises an electron-transport layer 403 adjacent to the cathode 201, a hole-injection/hole-transport layer comprising a hole-injection layer 404 adjacent to the anode 202 and at least one hole-transport layer 407 adjacent to the hole-injection layer 404. Between the electron-transport layer 403 and the hole-transport layer 407, the OLED further comprises an emitter layer 406 wherein holes and electrons recombine to produce light.

In yet another embodiment, the OLED comprises a hole-injection layer adjacent to the anode and at least two hole-transport layers, a first hole-transport layer adjacent to the hole-injection layer and a second hole-transport layer adjacent to the first hole-transport layer.

In one embodiment, the hole-injection layer and the at least two hole-transport layers are deposited separately. In another embodiment, at least two of the layers are inter-deposited.

In other embodiments, the OLED comprises an electron-injection layer and at least one electron-transport layer.

In yet another embodiment, the electroluminescent medium comprises a hole-injection/hole-transport layer adjacent to the anode, an electron-injection/electron-transport layer adjacent to the cathode, and an emitter layer between the hole-injection/hole-transport layer and the electron-injection/electron-transport layer.

In yet another embodiment, the OLED can further comprise an additional layer adjacent to the top electrode. In a preferred embodiment, the layer comprises indium tin oxide.

Other OLED structures will be evident to those skilled in the art.

In one embodiment, a typical OLED is formed by starting with a semi-transparent bottom electrode deposited on a glass substrate. In one embodiment, the electrode is an anode. In another embodiment, the electrode is a cathode. In another embodiment, the top electrode is semi-transparent.

An anode is typically about 800 Å thick and can have one layer comprising a metal having a high work function, a metal oxide and mixtures thereof. Preferably, the anode comprises a material selected from the group consisting of a conducting or semiconducting metal oxide or mixed metal oxide such as indium zinc tin oxide, indium zinc oxide, ruthenium dioxide, molybdenum oxide, nickel oxide or indium tin oxide, a metal having a high work function, such as gold or platinum, and a mixture of a metal oxide and a metal having a high work function. In one embodiment, the anode further comprises a thin layer (approximately 5-15 Å thick) of dielectric material between the anode and the first hole-injection/hole-transport layer. Examples of such dielectric materials include, but are not limited to, lithium fluoride, cesium fluoride, silicon oxide and silicon dioxide. In another embodiment, the anode comprises a thin layer of an organic conducting material adjacent to the hole-injection/hole-transport layer. Such organic conducting materials include, but are not limited to, polyaniline, PEDOT-PSS, and a conducting or semi-conducting organic salt thereof.

A semi-transparent cathode is typically between 70 and 150 Å thick. In one embodiment, the cathode comprises a single layer of one or more metals, at least one of which has a low work function. Such metals include, but are not limited to, lithium, aluminum, magnesium, calcium, samarium, cesium and mixtures thereof. Preferably, the low work function metal is mixed with a binder metal, such as silver or indium. In another embodiment, the cathode further comprises a layer of dielectric material adjacent to the electron-injection/electron-transport layer, the dielectric material including, but not limited to, lithium fluoride, cesium fluoride, lithium chloride and cesium chloride. Preferably, the dielectric material is lithium fluoride or cesium fluoride. In preferred embodiments, the cathode comprises either aluminum and lithium fluoride, a mixture of magnesium and silver, or a mixture of lithium and aluminum. In yet another embodiment, the cathode comprises magnesium, silver and lithium fluoride.

In one embodiment, the hole-injection/hole-transport layer is about 750 Å thick. In a preferred embodiment, the hole-injection/hole-transport material comprises a compound of formula 1. In a particularly preferred embodiment, the hole-injection/hole-transport layer comprises bis(N,N′-1-naphthyl-phenyl-amino-biphenyl)-trityl aniline (“TTA-DNPB”).

In one embodiment, an OLED comprises an emitter layer between the electron-injection/electron-transport layer and the hole-injection/hole-transport layer in which electrons from the electron-injection/electron-transport layer and holes from the hole-injection/hole-transport layer recombine. Depending on the composition of the emitter layer, OLEDs emit visible light of different colors. Emitter layers typically comprise at least one host compound, either alone or together with at least one dopant compound. Examples of host compounds include, but are not limited to, ALQ, IDE-120 and IDE-140 (Idemitsu Kosan Co., Ltd., Tokyo, Japan). Examples of dopant compounds include, but are not limited to, Coumarin 6, Coumarin 485, Coumarin, 487, Coumarin 490, Coumarin 498, Coumarin 500, Coumarin 503, Coumarin 504, Coumarin 504T, Coumarin 510, Coumarin 515, Coumarin 519, Coumarin 521, Coumarin 521T, Coumarin 522B, Coumarin 523, Coumarin 525, Coumarin 535, Coumarin 540A, Coumarin 545, quinacridone derivatives such as diethyl pentyl quinacridone and dimethyl quinacridone, distyrylamine derivatives, such as IDE-102, IDE-1O5 (Idemitsu Kosan Co., Ltd., Tokyo, Japan), rubrene, DCJTB, pyrromethane 546, and mixtures thereof. The structure of DCJTB is shown below:

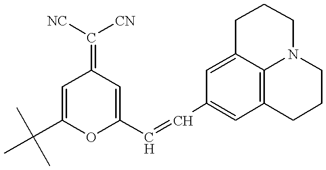

An emitter layer may be between 200-400 Å thick.

The electron-injection/electron-transport layer is typically about 350 Å thick and comprises a compound such as ALQ, or a suitable oxadiazole derivative. In a preferred embodiment, the electron-injection/electron-transport layer is ALQ.

In one preferred embodiment, an OLED of the present invention is a down-emitter that emits green light and comprises an anode comprising indium tin oxide, a hole-injection layer adjacent to the anode comprising a compound of formula 1, a hole-transport layer adjacent to the hole-injection layer comprising a compound of formula 1, an emitter layer adjacent to the hole-transport layer comprising ALQ and a compound selected from the group consisting of Coumarin 6, Coumarin 485, Coumarin, 487, Coumarin 490, Coumarin 498, Coumarin 500, Coumarin 503, Coumarin 504, Coumarin 504T, Coumarin 510, Coumarin 515, Coumarin 519, Coumarin 521, Coumarin 521T, Coumarin 522B, Coumarin 523, Coumarin 525, Coumarin 535, Coumarin 540A, Coumarin 545, and mixtures thereof, an electron-transport layer adjacent to the emitter layer comprising ALQ, and a cathode comprising either lithium fluoride and aluminum or magnesium and silver.

In another preferred embodiment, an OLED of the present invention is an up-emitter that emits green light and comprises an anode comprising molybdenum oxide, a hole-injection layer adjacent to the anode comprising a compound of formula 1, a hole-transport layer adjacent to the hole-injection layer comprising a compound of formula 1, an emitter layer adjacent to the hole-transport layer comprising ALQ and a compound selected from the group consisting of Coumarin 6, Coumarin 485, Coumarin, 487, Coumarin 490, Coumarin 498, Coumarin 500, Coumarin 503, Coumarin 504, Coumarin 504T, Coumarin 510, Coumarin 515, Coumarin 519, Coumarin 521, Coumarin 521T, Coumarin 522B, Coumarin 523, Coumarin 525, Coumarin 535, Coumarin 540A, Coumarin 545, and mixtures thereof, an electron-transport layer adjacent to the emitter layer comprising ALQ, and a cathode comprising lithium fluoride, magnesium and silver.

In yet another preferred embodiment, an OLED of the present invention emits white or blue light and comprises an anode comprising indium tin oxide, a hole-injection layer adjacent to the anode comprising a compound of formula 1, a hole-transport layer adjacent to the hole-injection layer comprising a compound of formula 1, an emitter layer adjacent to the hole-transport layer comprising DCJTB, IDE-102 and IDE-120, an electron-transport layer adjacent to the emitter layer comprising ALQ, and a cathode comprising lithium fluoride and aluminum.

In a preferred embodiment of the present invention, the OLED display device is a microdisplay. A microdisplay is a display device that is not viewable by the unaided eye, and therefore requires the use of an optic. Preferably, the sub-pixel size of a microdisplay device is less than about 15 microns, more preferably less than about 5 microns, and most preferably between about 2 microns and about 3 microns.

The multi-layered OLED devices of the invention allow for a “staircase” change in the energy difference of electrons and holes as they travel from the electrodes through each layer toward the emitter layer, where they recombine to emit light. Typically, the anode and cathode of an OLED have an energy difference of about 1.6-1.8 eV. A typical band gap of electrons and holes in the emitter layer is about 2.7 eV-2.9 eV, so that radiation emission resulting from recombination is in the visible light region (1.75 to 3 eV). In the present invention, the increase in energy difference of holes and electrons from the anode and cathode to the emitter layer is accomplished incrementally as the electrons and holes travel through the layers between the electrodes and the emitter layer. The energy difference is increased in increments of about 0.2-0.3 eV per layer to achieve the resulting band gap of 2.7 eV-2.9 eV in the emitter layer. A staircase change in energy provides for a lower operating voltage and better efficiency of operation of the OLED device, resulting in a higher quantum yield of luminescence for a given current density.

### HOLE-INJECTION AND HOLE-TRANSPORT MATERIALS

The present invention relates to a novel family of organic aromatic amine materials with high and tunable T_{g} values and tunable ionization potentials, which are useful as hole-transport and hole-injection materials in OLED display devices. The organic aromatic amine materials of the present invention are also useful as optoelectronic active elements in devices including, but not limited to, photocells, organic charge transfer devices, electrode surface modifications, fuel cells, electrochromic devices and optical limiting devices. In particular, the present invention relates to hole-injection and hole-transport materials comprising a compound of formula 1:

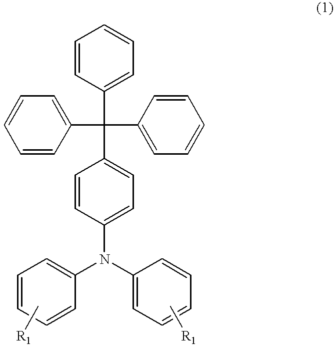

wherein each R₁ is independently selected from the group consisting of hydrogen, C₁-C₆ straight or branched chain alkyl, alkoxy, —CN, —Cl, —F, —CF₃, —SR, —SiR, vinyl that is unsubstituted or substituted at terminal carbon position(s) with C₁-C₆ alkyl or an aromatic group, aryl that is unsubstituted or substituted at para-, meta- or ortho- positions with a C₁-C₆ alkyl, C₁-C₈ alkoxy or —SR,

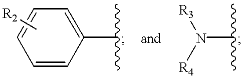

R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C₈ alkoxy or —SR, and

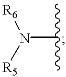

R₃, R_{4,} R₅ and R₆ are each independently selected from the group consisting of:

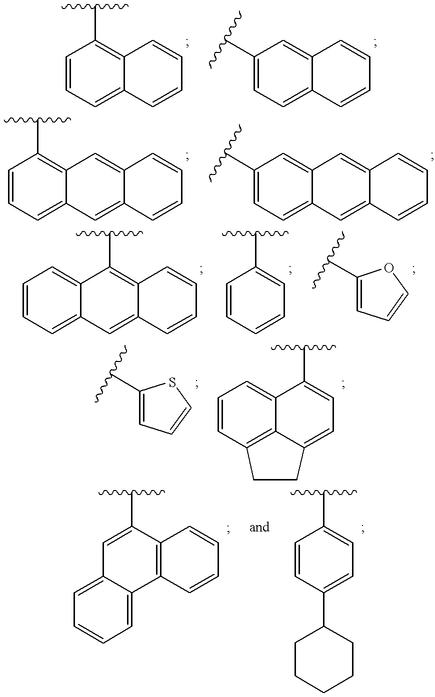

or R₃ and R₄ taken together with the nitrogen atom to which they are attached or R₅ and R₆ taken together with the nitrogen atom to which they are attached are independently selected from the group consisting of:

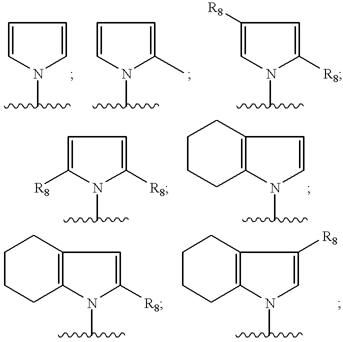

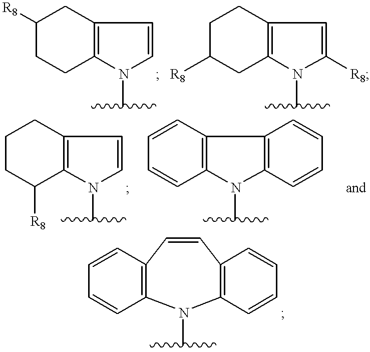

each R₈ is independently selected from the group consisting of —OR9, C₁-C4 alkyl aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

In one embodiment, the present invention relates to hole-injection and hole-transport materials comprising a compound of formula 1, wherein each R₁ is:

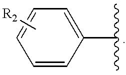

In another embodiment, the present invention relates to hole-injection and hole-transport materials comprising a compound of formula 1, wherein one of R₁ is:

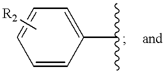

the other of R₁ is:

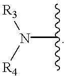

In yet another embodiment, the present invention relates to hole-injection and hole-transport materials comprising a compound of formula 1, wherein each of R₁ is:

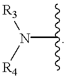

In yet another embodiment, the present invention relates to hole-injection and hole-transport materials comprising a compound of formula 1, wherein R₃ and R₄ taken together with the nitrogen atom to which they are attached or R₅ and R₆ taken together with the nitrogen atom to which they are attached are independently selected from the group consisting of:

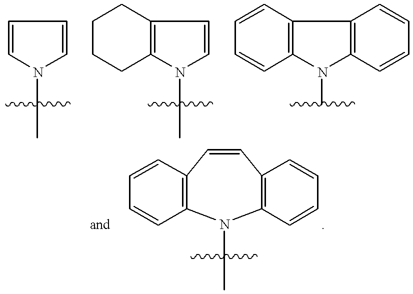

Additional compounds for this embodiment include compounds of formula 1, wherein R₃, R_{4,} R₅ and R₆ are each independently selected from the group consisting of:

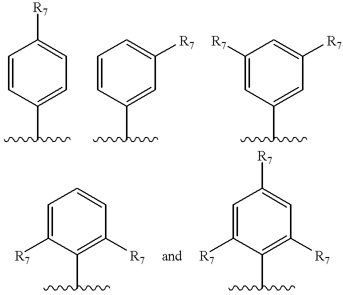

wherein each R₇ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Further additional compounds for this embodiment include compounds of formula 1, wherein R₃, R₄, R₅ and R₆ are each independently selected from the group consisting of:

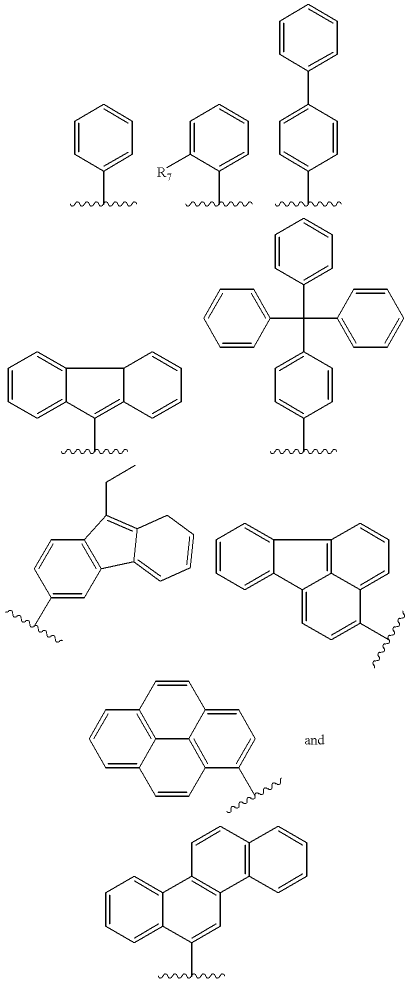

wherein each R₇ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

In a preferred embodiment, the present invention relates to hole-injection and hole-transport materials comprising a compound of formula 2:

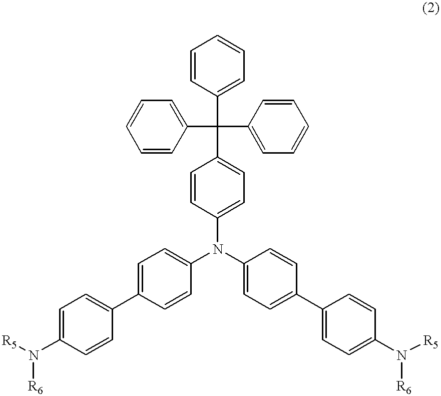

wherein R₅ and R₆ are each independently selected from the group consisting of

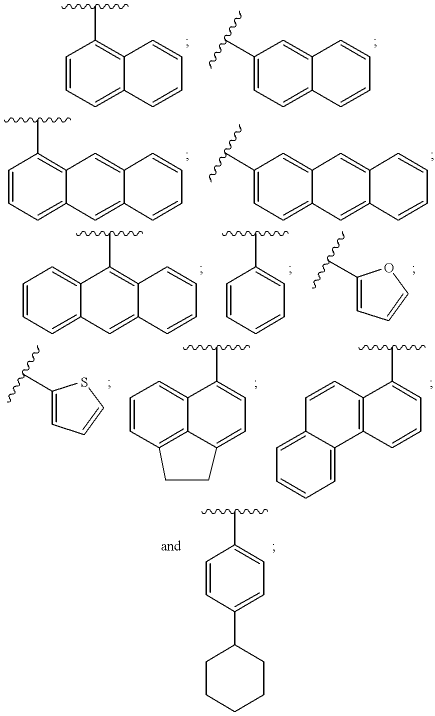

or R₅ and R₆ taken together with the nitrogen atom to which they are attached are independently selected from the group consisting of:

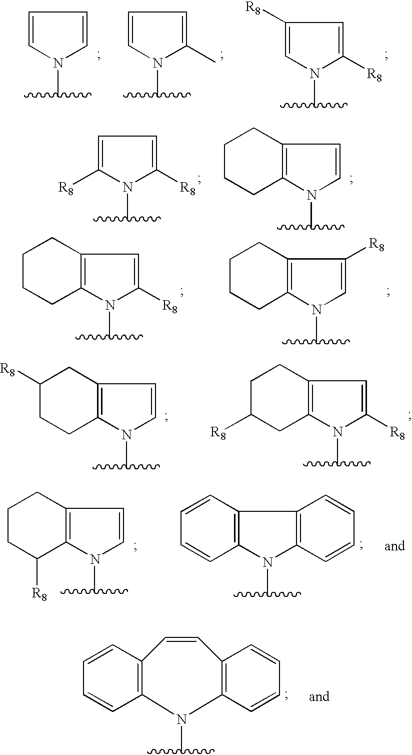

each R₈ is independently selected from the group consisting of —OR9, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Additional compounds for this embodiment include compounds of formula 2, wherein R₅ and R₆ are each independently selected from the group consisting of:

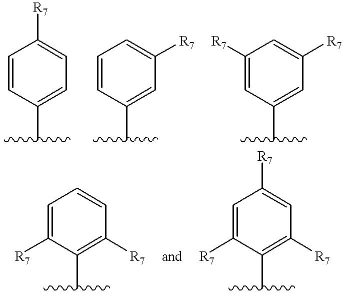

wherein each R₇ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Further additional compounds for this embodiment include compounds of formula 2, wherein R₅ and R₆ are each independently selected from the group consisting of:

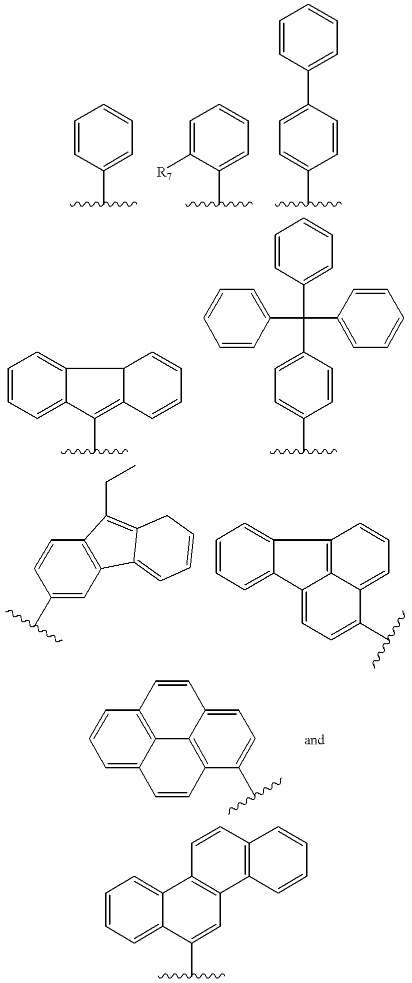

wherein each R₇ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

In another preferred embodiment, the present invention relates to hole-injection and hole-transport materials comprising a compound of formula 3:

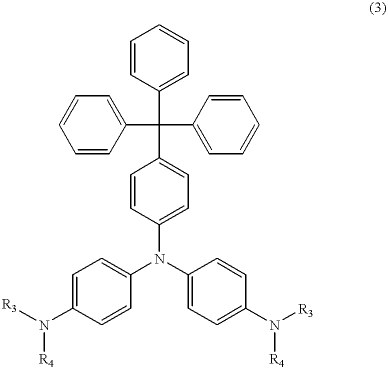

wherein R₃ and R₄ are each independently selected from the group consisting of:

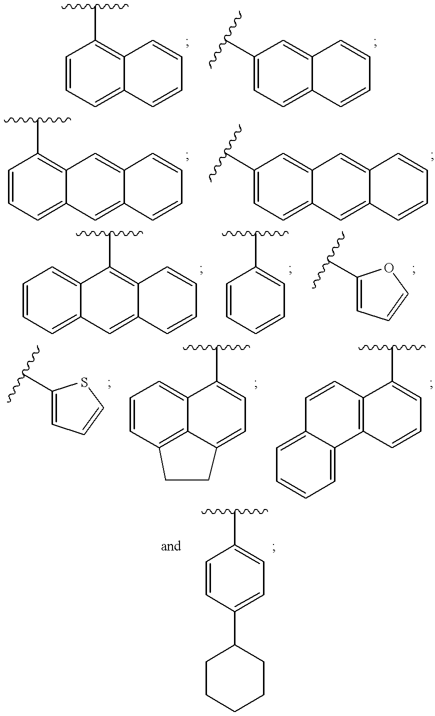

or R₃ and R₄ taken together with the nitrogen atom to which they are attached are selected from the group consisting of

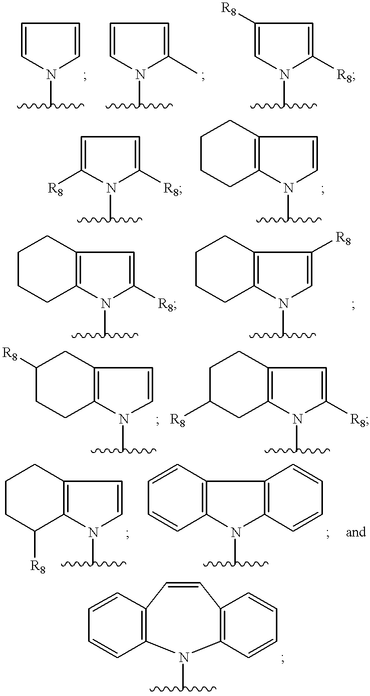

each R₈ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Additional compounds for this embodiment include compounds of formula 3, wherein R₃ and R₄ are each independently selected from the group consisting of:

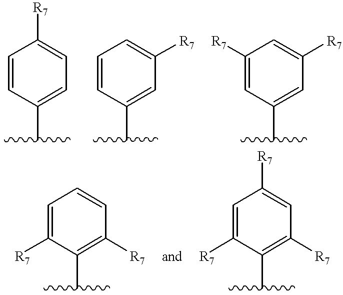

wherein each R₇ is independently selected from the group consisting of —OR₉, C₁-C₄ alkyl, aryl, —SCH₃, —CF₃, —Cl, —Br, —NO₂, —SR, SiR and —COOR₉;

R is C₁-C₆ straight or branched chain alkyl; and

R₉ is selected from the group consisting of C₁-C₆ alkyl and aryl.

Further additional compounds for this embodiment include compounds of formula 3, wherein R₃ and R₄ are each independently selected from the group consisting of:

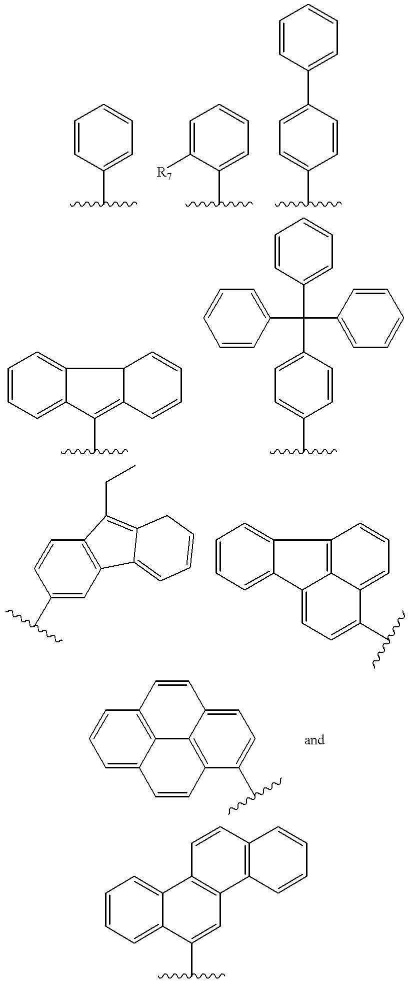

wherein each R₇ is independently selected from the group consisting of —OR₉, C₁-C₄ alkyl, aryl, —SCH₃, —CF₃, —Cl, —Br, —NO₂, —SR, SiR and —COOR₉;

R is C₁-C₆ straight or branched chain alkyl; and

R₉ is selected from the group consisting of C₁-C₆ alkyl and aryl.

In yet another preferred embodiment, the present invention relates to hole-injection and hole-transport materials comprising a compound of formula 4:

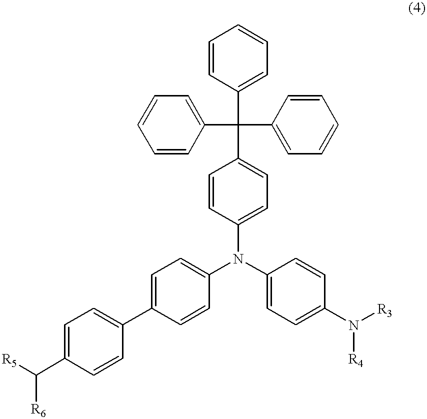

R₃, R₄, R₅ and R₆ are each independently selected from the group consisting of:

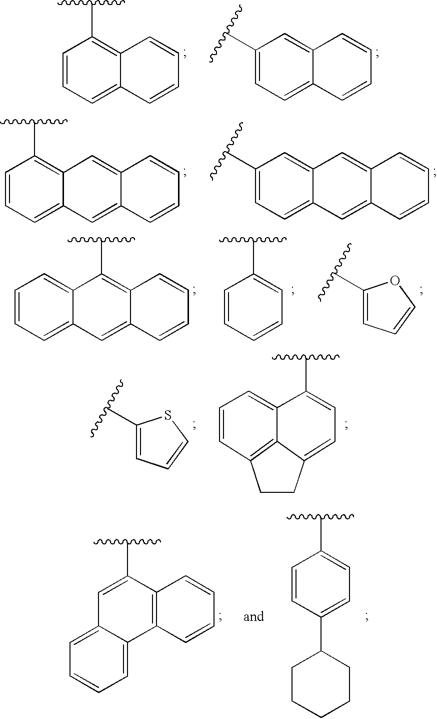

or R₃ and R₄ taken together with the nitrogen atom to which they are attached, or R₅ and R₆ taken together with the nitrogen atom to which they are attached are each independently selected from the group consisting of:

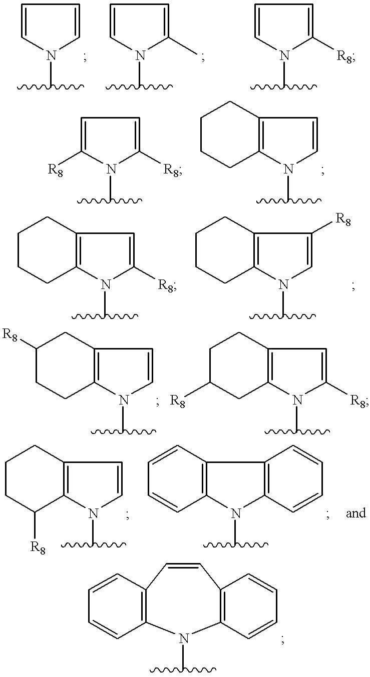

each R₈ is independently selected from the group consisting of

—OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Additional compounds for this embodiment include compounds of formula 4, wherein R₃, R_{4,} R₅ and R₆ are each independently selected from the group consisting of:

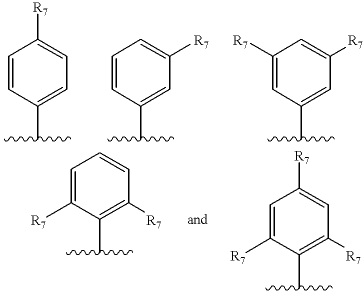

wherein each R₇ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Additional compounds for this embodiment include compounds of formula 4, wherein R₃, R₄, R₅ and R₆ are each independently selected from the group consisting of:

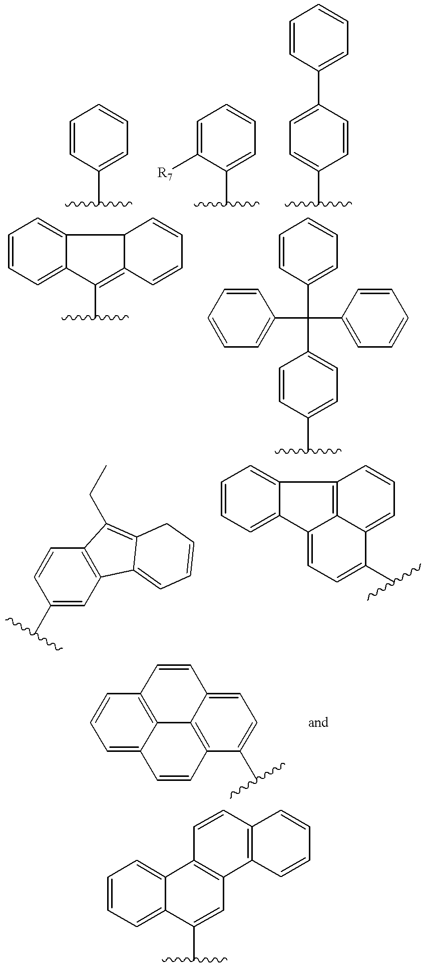

wherein each R₇ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

In one embodiment, the compound of formula 1 has the structure:

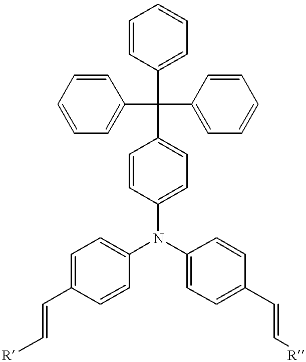

wherein each R′ and R″ is independently selected from the group consisting of hydrogen, C₁-C₆ alkyl, unsubstituted C₆-C₁₈ aryl, C₆-C₁₈ aryl that is substituted with C₁-C₆ alkyl, C₁-C₆ alkoxy or C₁-C₆ dialkyl amine, and C₅-C₁₈ aromatic or non-aromatic nitrogen-, oxygen- or sulfur-containing heterocyclic group.

The present invention also relates to OLEDs having hole-injection and hole-transport materials comprising a compound of formula 1.

In a preferred embodiment, the present invention relates to OLEDs having hole-injection and hole-transport materials comprising a compound of formula 2.

In another preferred embodiment, the present invention relates to OLEDs having hole-injection and hole-transport materials comprising a compound of formula 3.

In yet another preferred embodiment, the present invention relates to OLEDs having hole-injection and hole-transport materials comprising a compound of formula 4.

The organic aromatic amines of the present invention possess a Y-shaped molecular geometry. In contrast to the aforementioned four families of hole-injection and hole-transport materials (i.e., linear oligomer, starburst molecules, fluorene-based and spiro-bifluorene based aromatic amines), a Y-shaped molecular architecture may combine the advantages of linear oligomer with those of starburst molecules. Possible advantages and special features of this Y-shaped molecular architecture system include, but are not limited to: the availability of a wide selection of possible novel hole-injection and hole-transport materials having this geometry; high and tunable T_{g} values; suitable and tunable ionization potential values; suitable and tunable mobility when used as element(s) in optoelectronic devices; good thermal, photochemical and electrochemical stability; desirable band gap to provide better energy level matching with adjacent layers; materials are amenable to vapor deposition in order to form amorphous and robust thin films; and desirable molecular architecture to prevent the formation of inefficient exciplexes in OLED devices.

The T_{g} and ionization potentials of the compounds of formula 1 can be tuned by changing the chemical nature of the substituents (R₁-R₇). For example, functional groups on aryl rings attached to nitrogen atoms may be chosen to be electron donating groups, such as alkyl groups and phenyl groups, or they may be chosen to be electron withdrawing groups, such as fluorine containing groups (e.g., trifluoromethane). The compounds of formula 1 that are used as hole-injection and hole-transport materials in OLED devices typically possess three nitrogen atom centers, which may be separated from each other by phenyl or biphenyl groups. The nature of these linkages affects the charge transfer between the nitrogen centers, thus changing the band gap of the molecules. For example, if the nitrogen centers are separated by biphenyl groups, the effective charge transfer between the centers is more difficult, and therefore, the band gap of the hole-injection/hole-transport molecules is increased.

It is important to develop new optoelectronic active organic aromatic amine materials with high and tunable T_{g} values, good thermal stability, desirable band gap, and suitable ionization potentials. First, these new materials can be used as thermally stable hole-transport or hole-injection materials in OLED display devices. The incorporation of such hole-injection and hole-transport materials into display devices allows for storage and operation of OLED devices at elevated temperatures. For example, the Y-shaped compound BPA-DNPB has a T_{g} value of 140° C., which is 45 degrees higher than the 96° C. T_{g} value for NPB. The Y-shaped compound TTA-BCA has a T_{g} value of 171° C., which is 75 degrees higher than the 96° C. T_{g} value for NPB. Second, the incorporation of the compounds of formula 1 into OLED devices enables the direct patterning of color filters or color changing media on top of the devices at high temperatures. Third, the incorporation of the compounds of formula 1 into hole-injection and hole-transport layers of OLEDs allows them to be sealed at relatively high temperatures. Fourth, in some embodiments, the Y-shaped molecular geometry having three nitrogen centers and two biphenyl linkers assures the formation of high quality amorphous thin films and prevents the formation of a crystalline phase in these materials. Finally, the favorable hole-transport properties of the compounds of formula 1 allows them to be used in other devices, including, but not limited to, photocells, fuel cells, charge transfer devices, electrochromic devices and optical limiting devices.

Other Y-shaped compounds useful as hole-injection and hole-transport materials in OLED devices of the present invention include compounds of formula 5:

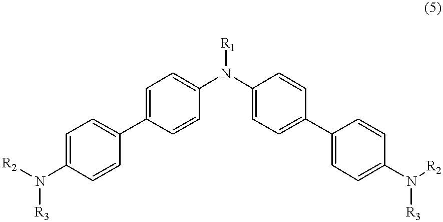

wherein R₁ is selected from the group consisting of:

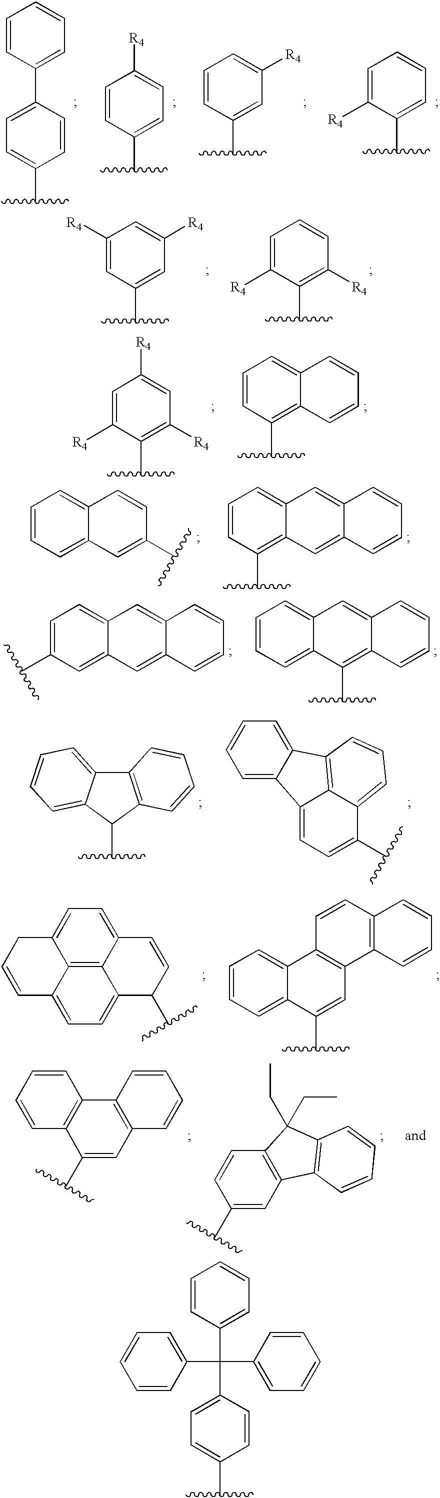

R₂ and R₃ are each independently selected from the group consisting of

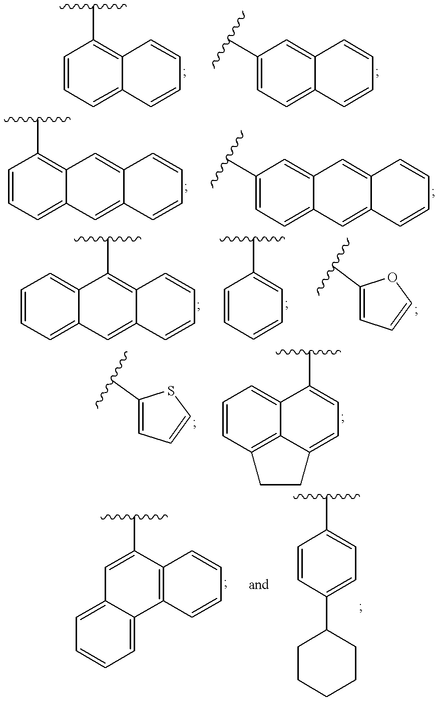

or R₂ and R₃ taken together with the nitrogen to which they are attached are selected from the group consisting of:

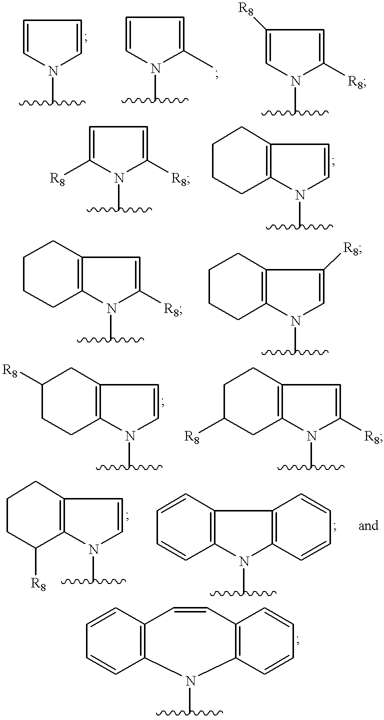

each R₄ is independently selected from the group consisting of —OR₉, C₁-C₄ alkyl, aryl, —SCH₃, —CF₃, —Cl, —Br, —NO₂, —SR, —SiR and —COOR₉;

each R₈ is independently selected from the group consisting of —OR₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Additional compounds that are useful in OLED devices of the present invention include compounds of formula 5 wherein R₂ and R₃ are independently selected from the group consisting of:

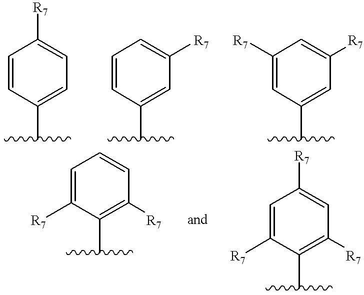

wherein each R₇ is independently selected from the group consisting of R₉, C₁-C₄ alkyl, aryl, —SCH₃, —CF₃, —Cl, —Br, —NO₂, —SR, —SiR and —COOR₉;

R is C₁-C₆ straight or branched chain alkyl; and

R₉ is selected from the group consisting of C₁-C₆ alkyl and aryl.

Further additional compounds that are useful in OLED devices of the present invention include compounds of formula 5 wherein R₂ and R₃ are independently selected from the group consisting of:

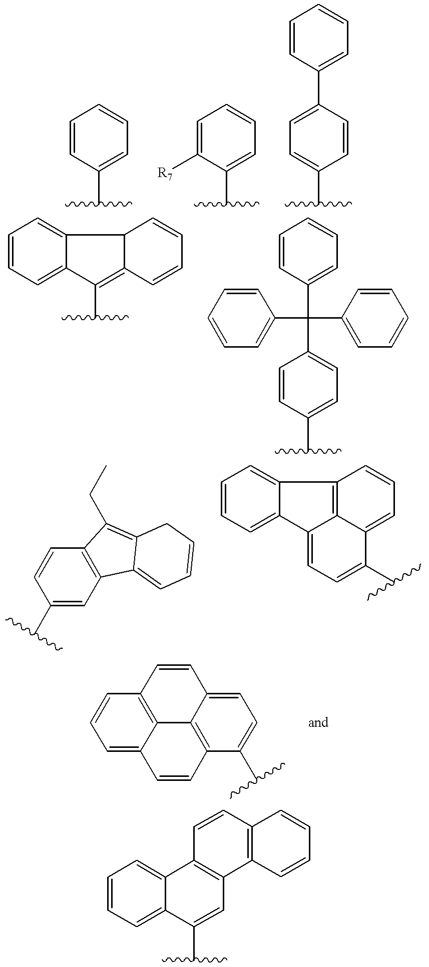

Y-shaped hole-injection and hole-transport compounds useful in OLED devices of the present invention include, but are not limited to, the structures shown in Table II:

TABLE II

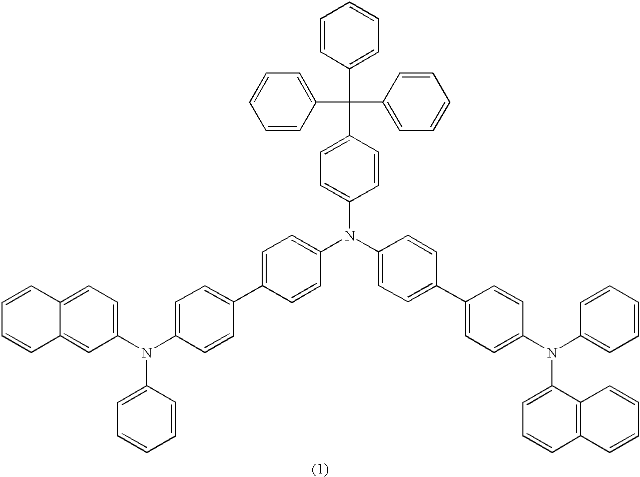

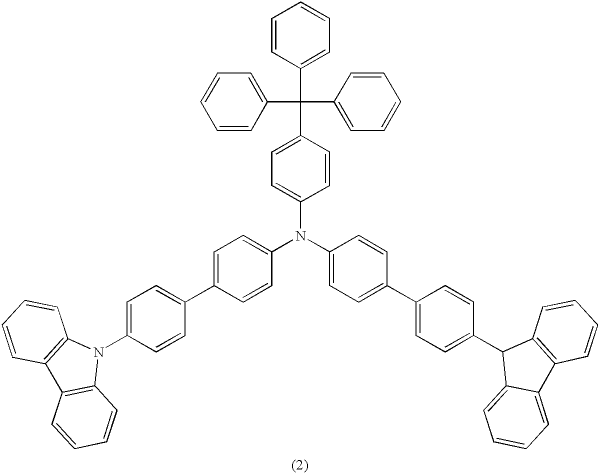

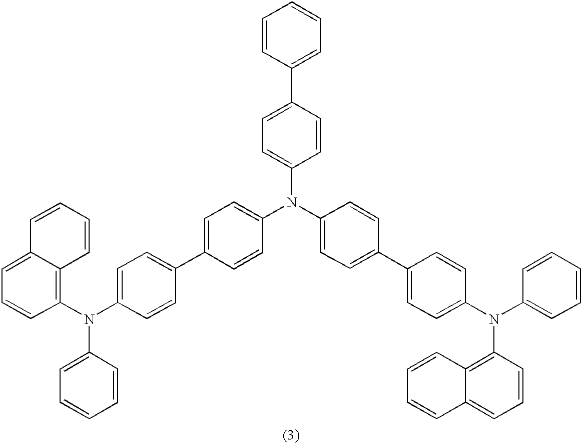

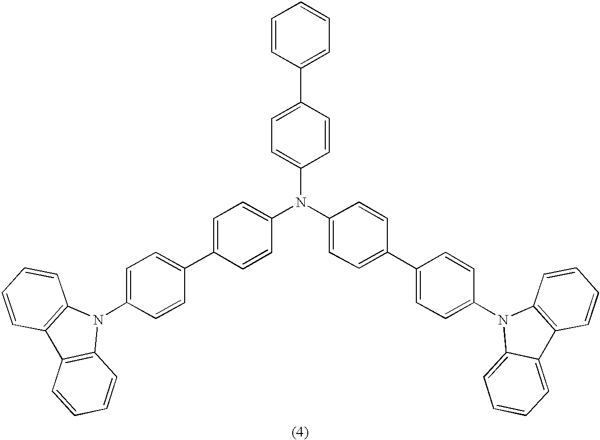

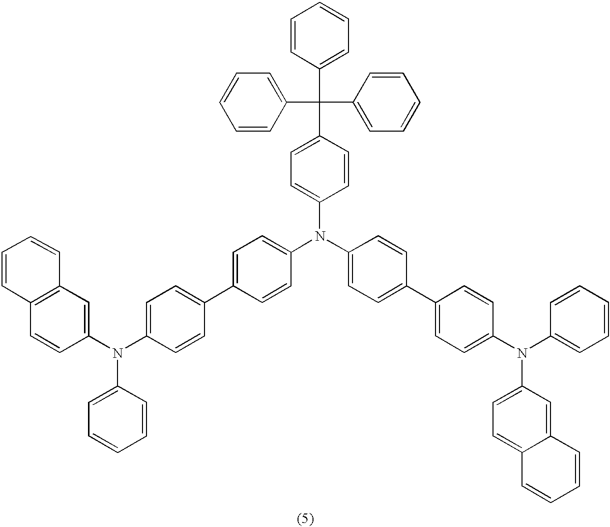

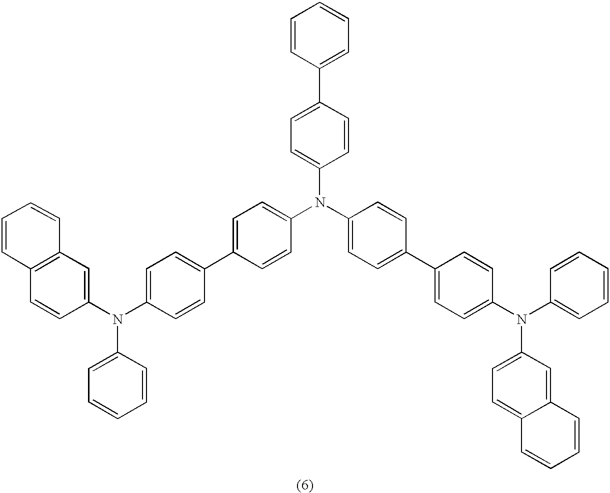

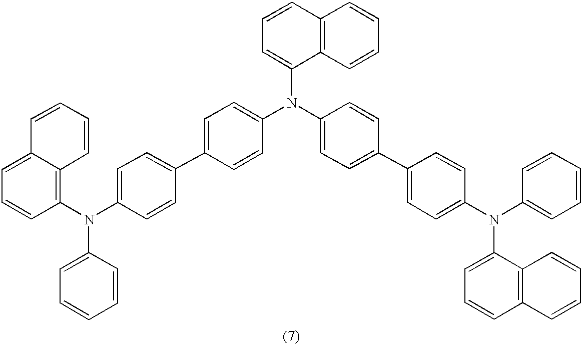

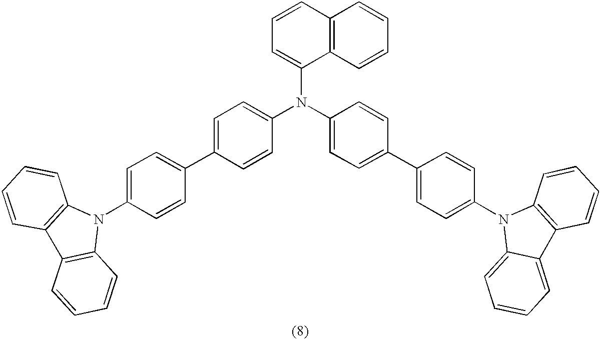

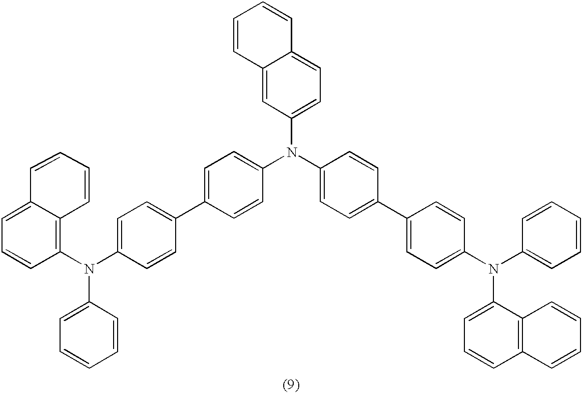

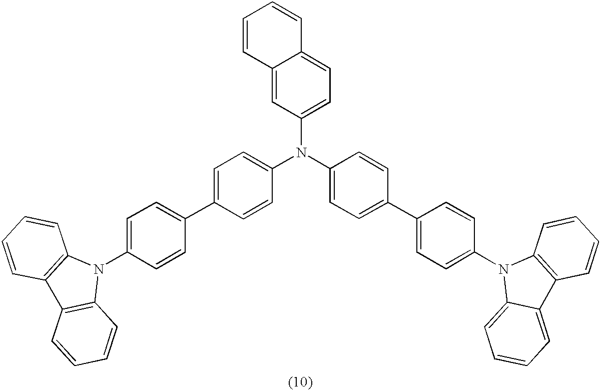

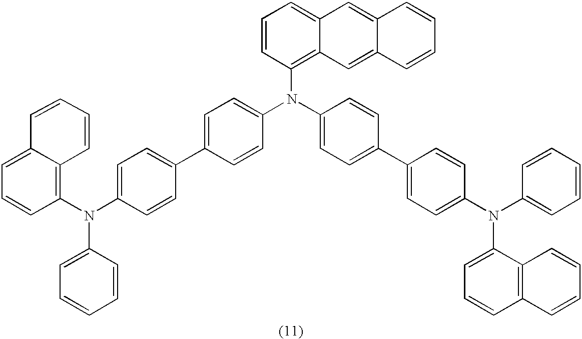

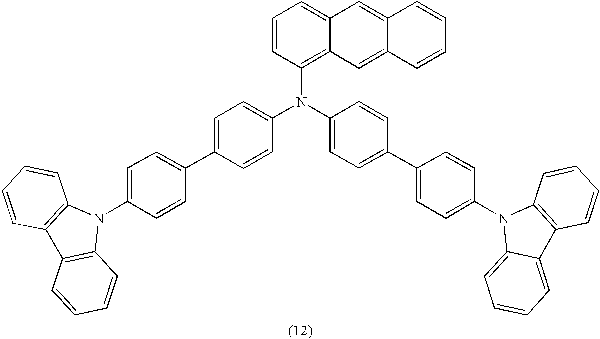

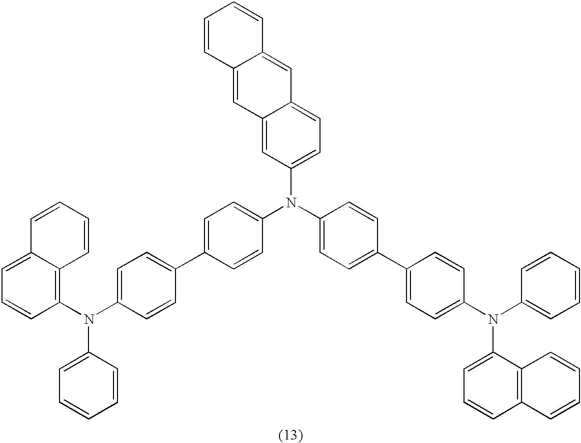

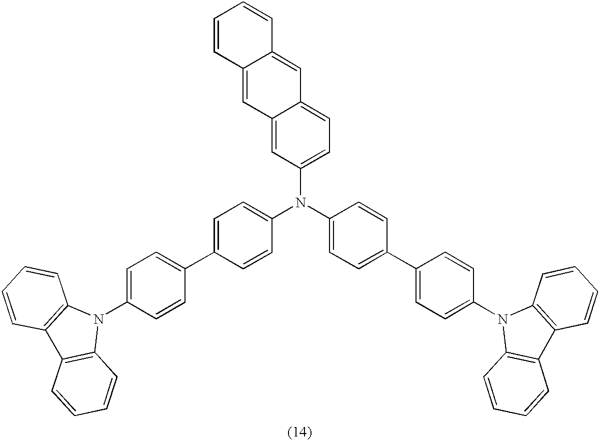

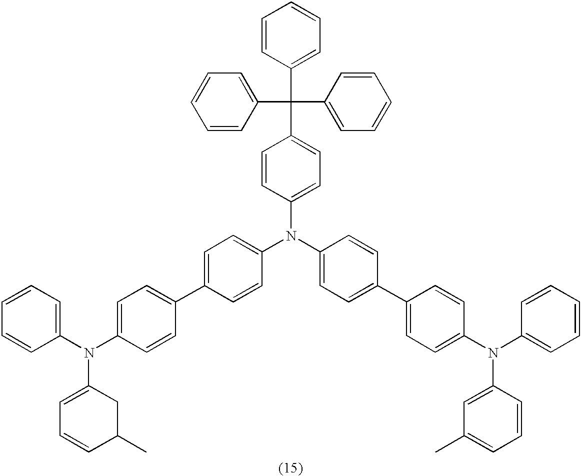

Particularly preferred among the compounds of Table II are numbers 1, 2, 5 and 15.

### SYNTHESIS OF HOLE-INJECTION AND HOLE-TRANSPORT COMPOUNDS

Hole-injection and hole-transport compounds of the present invention can be synthesized as shown in Scheme I.

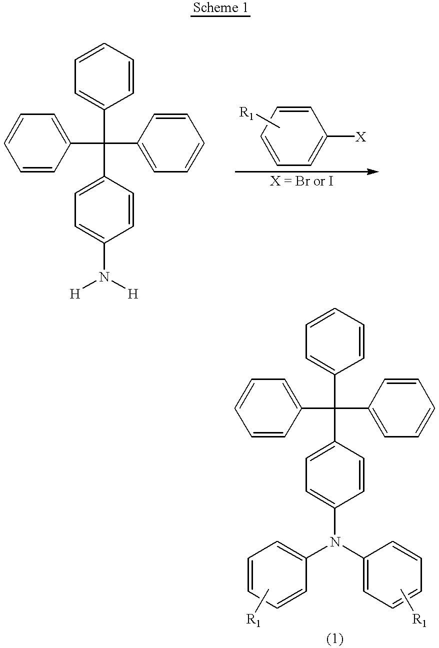

In this reaction, in an inert atmosphere dry box, about 0.5 equivalents of diphenylphosphino ferrocene (DPPF) and about 0.35 equivalents of tris(dibenzylideneacetone) dipalladium (Pd₂(dba)₃) are added to a solution comprising about 10 equivalents of trityl aniline, about 25 equivalents of a substituted or unsubstituted 4-bromophenyl or 4-iodophenyl compound and about 22 equivalents of sodium tert-butoxide in anhydrous toluene. The reaction mixture is then heated to about 95° C. for about 10 hours. Upon completion of the reaction, the solution is cooled to room temperature, organic solvent is removed by rotary evaporation and a compound of formula 1 is isolated by silica gel chromatography using gel of 230-400 mesh and hexane as the eluant. Reaction yield is about 85% using trityl aniline as the primary aromatic amine building block. Mass spectroscopic analysis may be used to confirm the formation of the compound of formula 1. The thermal properties and glass transition temperatures of compounds of formula 1 may be determined using differential scanning calorimetry (DSC) and thermo gravimetric analysis (TGA).

Where each R₁ is independently

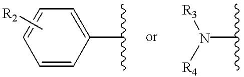

and R₂ is

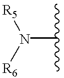

the compounds of formula 1 can be synthesized as shown in Scheme 2.

[chemical structure]

In Scheme 2, a first step is the formation of a compound of formula 6 or formula 7 or formula 8, which can be accomplished under similar conditions to those described for the formation of a compound of formula 1 in Scheme 1.

In Scheme 2, a compound of formula 1 is made in a second step from the compound of formula 6 or 7 or 8. In this step in an inert atmosphere dry box, catalytic amounts of DPPF and Pd₂(dba)₃ are added to a solution of the compound of formula 6 or 7 or 8 and sodium tert-butoxide dissolved in anhydrous toluene. To this solution is added 4 equivalents of a secondary aromatic amine dissolved in toluene. The reaction mixture is heated to about 95° C. for about 10 hours. Upon completion of the reaction, the solution is cooled to room temperature, organic solvent is removed by rotary evaporation and a compound of formula 1 is isolated by silica gel chromatography. Reaction yields range from 75% to 95%. The product may be further purified by sublimation. Elemental and mass spectroscopic analyses may be used to confirm the formation of the compounds of formula 1.

In one embodiment, a compound of formula 1 can be made from a compound of formula 8 in one step by mixing NH(R₃)(R₄) and NH(R₅)(R₆) with a compound of formula 8. In another embodiment, a compound of formula 1 can be made from a compound of formula 8 in a step-wise fashion by making a secondary amine with NH(R₃)(R₄) first and then coupling the secondary amine with NH(R₅)(R₆) in a subsequent step.

The thermal properties and glass transition temperatures of compounds of formula 1 may be determined using differential scanning calorimetry (DSC) and thermo gravimetric analysis (TGA).

The compounds of formulas 2, 3 and 4 may also be synthesized according to Scheme 2.

The compounds of formula 5 may be synthesized according to Scheme 3 using the same two steps as discussed for the synthesis of the compounds of formula 1 in Scheme 2.

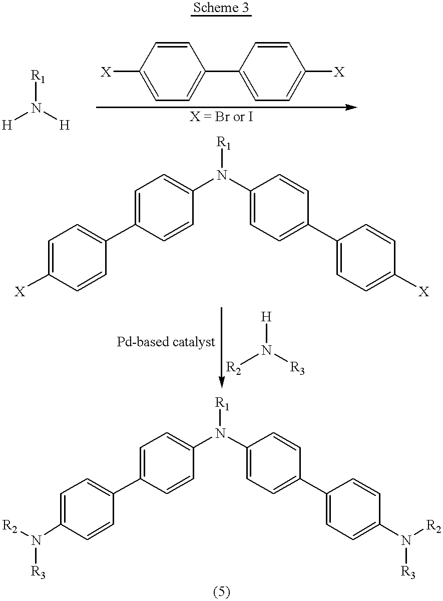

The synthesis and purification of the compounds of formulas 1-5 as shown in Schemes 1, 2 and 3 may be easily scaled-up in order to provide adequate amounts of hole-injection and hole-transport materials for OLED device fabrication and for other potential applications.

Syntheses of the compounds of formulas 1-4 employ the primary aromatic amine trityl aniline as a starting material. Trityl aniline is a primary amine attached to a highly symmetrical tetrahedral-shaped trityl group. The primary amine group at one of the four para positions of the phenyl groups can be selectively functionalized to allow for a wide variety of organic units to be assembled asymmetrically on one side of the tetrahedral core. The rigid tetrahedral geometry of the trityl group may reduce the possibility of formation of intermolecular x-stacked complexes of the compounds of formulas 1-4. The presence of aromatic amine moieties in these newly designed molecules assures that the materials will possess suitable ionization potentials and good hole-injection/hole-transport properties.

Two examples of compounds of formula 1 are TTA-DNPB and TTA-BCA. In the synthesis of TTA-DNPB, as shown below in Example 1, two NPB-like motifs were attached to the nitrogen atom of trityl aniline. As shown below, 4,4′-bis-halogenated-biphenyl molecules, such as 4,4′-dibromobiphenyl or 4,4′-diiodobiphenyl, can be used as linkers in the syntheses of these molecules in order to prepare the molecular intermediates having a tertiary amine center covalently connected to two 4-bromo- or 4-iodo-biphenyl groups. The subsequent coupling reaction of any selected secondary aryl amine with the intermediate at the two halogen atom positions yields the desired aromatic amines as new optoelectronic active materials. Both steps of the coupling reactions may be accomplished using a palladium-based catalyst.

In one embodiment, the compounds of formula 1 comprise three nitrogen atom centers, which may be separated from each other by phenyl or biphenyl groups. In TTA-DNPB and TTA-BCA, and in the compounds of formulas 2 and 5, the three nitrogen atoms are separated from each other by two biphenyl groups, which decreases the effective charge transfer between the different nitrogen atom centers and increases the band gap of the molecules. Some research results have indicated that more thermally stable hole-injection/hole-transport materials can be obtained by using a biphenyl link between nitrogen atoms in an aromatic amine molecule. Thus, these new hole-injection/hole-transport molecules are predicted to have good thermal stability, relatively higher glass transition temperatures, and suitable ionization potentials.

Synthesis of the compounds of formula 5 employs a variety of primary aromatic amine starting materials. These primary aromatic amines may include unsubstituted amines, such as aniline, or substituted primary amines, such as mono-, bis- or tri-substituted aniline. These primary aromatic amines may also include primary amines with fused aromatic rings, such as naphthyl amine and fluorene amines. Thus, functional groups on the primary amine starting material may be electron donating groups, such as alkyl groups and phenyl groups, or they may be electron withdrawing groups, comprising fluorine atoms and trifluoromethyl groups.

T_{g} and ionization potential values of the compounds of formulas 1-5 may be adjusted by varying the nature of the functional groups at each position. Thus, a large number of hole-injection and hole-transport compounds can be made. For example, the total number of hole transport materials that are compounds of formula 5 is equal to R₁×R₂ (or R₃) (=R₁R₂ or R₁R₃).

### EXAMPLES EXAMPLE 1: SYNTHESIS OF BIS(N,N′-1-NAPHTHYL-PHENYL-AMINO-BIPHENYL)-TRITYL ANILINE (TTA-DNPB)

### MATERIALS

Silica gel having average particle size of 230-400 mesh from Whatman was used in a 20 cm column for purification. Compounds were eluted using 5% CH₂Cl₂ in hexane as the mobile phase.

Sublimation was performed using a train sublimation apparatus designed in the laboratory at a pressure of 1.0×10⁶ torr and at temperature of 350° C.

Mass spectroscopy was performed on a SFNNIGAN 4500 instrument from Sfnnigan Corporation using direct ionization with methane as the gas at a pressure of 0.4 millitorr.

TGA was performed on a TGA-50 instrument from Shimadzu.

DSC was performed using a DSC-50 instrument from Shimadzu.

All starting materials and solvents for the syntheses were of pure grade and were used without further purification.

### METHODS

In an inert atmosphere box, catalytic amounts of diphenylphosphino ferrocene (DPPF) (0.5 eq) and tris(dibenzylideneacetone) dipalladium (Pd₂(dba)₃) (0.35 eq) were added to a solution of 10 eq of trityl amine, 25 eq of 4,4′-dibromobiphenyl and 10.5 g (22 eq) of sodium tert-butoxide in anhydrous toluene. The reaction mixture was heated at 95° C. for 10 hours. The reaction solution was cooled to room temperature, organic solvent was removed by rotary evaporation, and tritylaniline-bis-biphenyl bromide (TTA-BPBBr) (9) was isolated by silica gel chromatography (see Materials, above). The reaction yield was 83.5%. Mass spectroscopic analysis (see Materials, above) confirmed the formation of title compound, TTA-BPBBr (9) (calculated molecular weight=797.6 g/mol, measured molecular weight=797.1 g/mol).

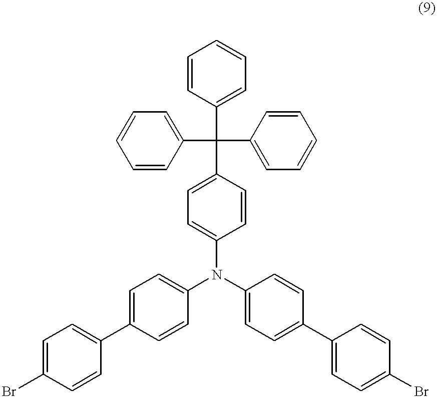

In an inert atmosphere box, 0.35 mmol (320 mg) of Pd₂(dba)₃ and 0.5 mmol (280 mg) of DPPF were added to a solution of 2 mmol (1.60 g) of TTA-BPBBr (9) and 5.5 mmol (0.85 g) of sodium tert-butoxide dissolved in 30 mL of anhydrous toluene. Five mmol (1.10 g) of phenylnaphthyl dissolved in 10 mL of toluene were added to this solution. The reaction mixture was heated at 95° C. for 30 hours. Upon completion of the reaction, the solution was cooled to room temperature, organic solvent was removed by rotary evaporation, and the product TTA-DNPB (10) was isolated by silica gel chromatography (see Materials, above). The reaction yield was 83%. The product was further purified by sublimation. Mass spectroscopic analysis (see Materials, above) confirmed the formation of TTA-DNPB (10) (calculated molecular weight=1074.3 g/mol, measured molecular weight=1073.4 g/mol). Differential scanning calorimetry analysis (see Materials, above) from 50 to 320° C. revealed a glass transition temperature of about 155° C. (one of the highest reported T_{g} values for a hole-injection/hole-transport molecule).

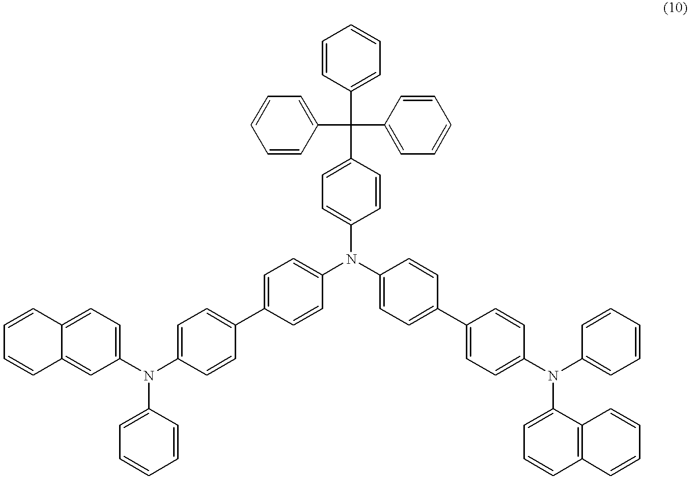

### EXAMPLE 2: SYNTHESIS OF BIS(CARBAZOL-N-BIPHENYL)-1-TRITYL ANILINE (TTA-BCA)

TTA-BPBBr was synthesized as described above in Example 1.

In an inert atmosphere dry box, 0.35 mmol (320 mg) of Pd₂(dba)₃ and 0.5 mmol (280 mg) of DPPF were added to a solution of 2 mmol (1.60 g) of TTA-BPBBr (9) and 5.5 mmol (0.85 g) of sodium tert-butoxide dissolved in 30 mL of anhydrous toluene. Five mmol (0.85 g) of carbazole dissolved in 10 mL of toluene were added to this solution. The reaction mixture was heated at 95° C. for 30 hours. Upon completion of the reaction, the solution was cooled to room temperature, organic solvent was removed by rotary evaporation, and the product TTA-BCA (11) was isolated by silica gel chromatography (see Materials, Example 1, above). The reaction yield was about 80%. The product was further purified by sublimation. Mass spectroscopic analysis (see Materials, Example 1, above) confirmed the formation of TTA-BCA (11) (calculated molecular weight=970.2 g/mol, measured molecular weight=970.5 g/mol). The T_{g} value was determined by differential scanning calorimetry (see Materials, Example 1, above) from 50 to 320° C. to be about 171° C., also one of the highest T_{g} values reported for a hole-injection/hole-transport material.

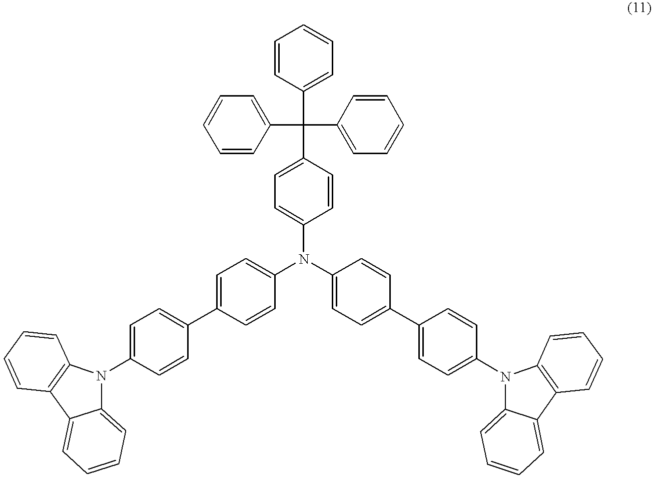

### EXAMPLE 3: SYNTHESIS OF BIS(CARBAZOL-N-BIPHENYL)-BIPHENYL AMINE (BPA-BCA)

Due to the extremely high T_{g} values of TTA-DNPB and TTA-BCA, it was more difficult to form thin films of these materials by vacuum deposition. Therefore, 4-aminobiphenyl was also used instead of trityl aniline as the primary amine building block to generate BPA-BCA, which has a lower T_{g}.

### METHODS

In an inert atmosphere box, catalytic amounts of diphenylphosphino ferrocene (DPPF) (285 mg) and tris(dibenzylideneacetone) dipalladium (Pd₂(dba)₃) (312 mg) were added to a solution of 0.85 g (1 eq) of diphenylamine, 4.70 g (3 eq) of 4,4′-dibromobiphenyl and 1.05 g (2.2 eq) of sodium tert-butoxide in anhydrous toluene. The reaction mixture was heated at 95° C. for 30 hours. The reaction solution was cooled to room temperature, organic solvent was removed by rotary evaporation, and biphenylamino-bis-biphenyl bromide (BPA-BPBBr) (12) was isolated by silica gel chromatography (see Materials, above). 2.80 g (0.89 eq) of BPA-BPBBr was obtained after separation, giving a reaction yield of 89%. Mass spectroscopic analysis (see Materials, above) confirmed the formation of BPA-BPBBr (12).

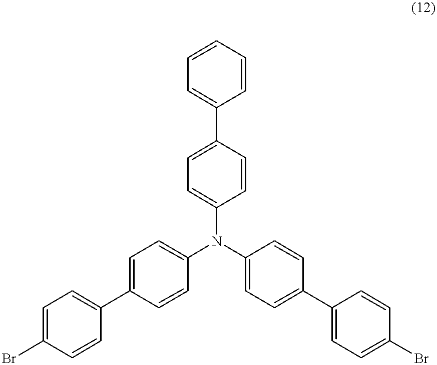

In an inert atmosphere box, 0.35 mmol (320 mg) of Pd₂(dba)₃ and 0.5 mmol (280 mg) of DPPF were added to a solution of 2 mmol (1.26 g) of BPA-BPBBr (12) and 5.5 mmol (0.55 g) of sodium tert-butoxide dissolved in 25 mL of anhydrous toluene. 5.0 mmol (0.85 g) of carbazole dissolved in 20 mL of toluene were added to this solution. The reaction mixture was heated at 95° C. for 30 hours. Upon completion of the reaction, the solution was cooled to room temperature, organic solvent was removed by rotary evaporation, and the product BPA-BCA (13) was isolated by silica gel chromatography (see Materials, above). 1.29 g of crude product was obtained (80% yield). BPA-BCA was further purified by sublimation (see Materials, above). Mass spectroscopic (see Materials, above) analysis confirmed the formation of BPA-BCA (calculated molecular weight=804.0 g/mol, measured molecular weight=804.0 g/mol). The glass transition temperature (T_{g}) was determined by DSC (see Materials, above) to be about 162° C. Thus, BPA-BCA should be more easily sublimated than TTA-BCA during fabrication of OLED devices.

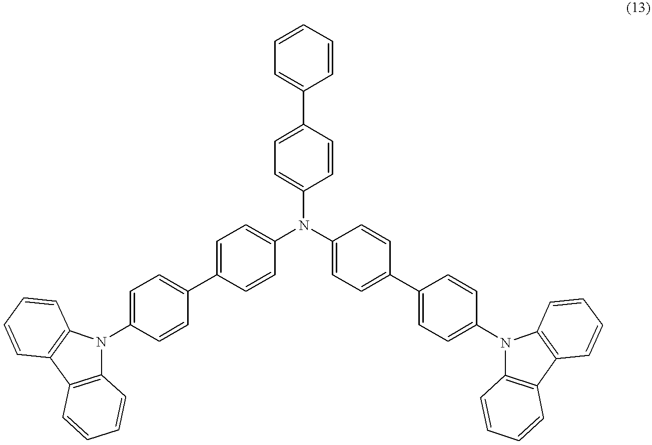

### REFERENCES CITED

All references cited herein are incorporated herein by reference in their entirety and for all purposes to the same extent as if each individual publication or patent or patent application was specifically and individually indicated to be incorporated by reference in its entirety for all purposes as fully set forth.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A hole-injection or hole-transport compound of formula 1: 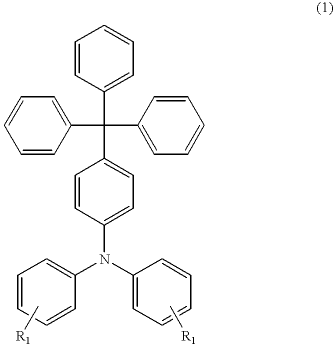
wherein each R₁ is independently selected from the group consisting of hydrogen, C₁-C₆ straight or branched chain alkyl, alkoxy, —CN, —Cl, —F, —CF₃, —SR, —SiR, vinyl that is unsubstituted or substituted at terminal carbon position(s) with C₁-C₆ alkyl or an aromatic group, aryl that is unsubstituted or substituted at para-, meta- or ortho- positions with a C₁-C₆ alkyl, C₁-C₈ alkoxy or —SR, 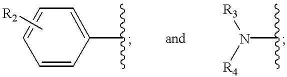
R₂ is selected from the group consisting Of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C8 alkoxy or —SR, and 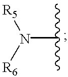
R₃, R_{4,} R₅ and R₆ are each independently selected from the group consisting of: 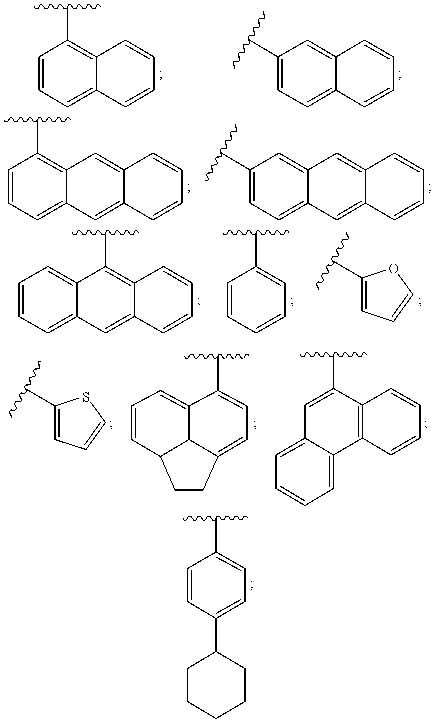
or R₃ and R₄ taken together with the nitrogen atom to which they are attached or R₅ and R₆ taken together with the nitrogen atom to which they are attached are independently selected from the group consisting of: 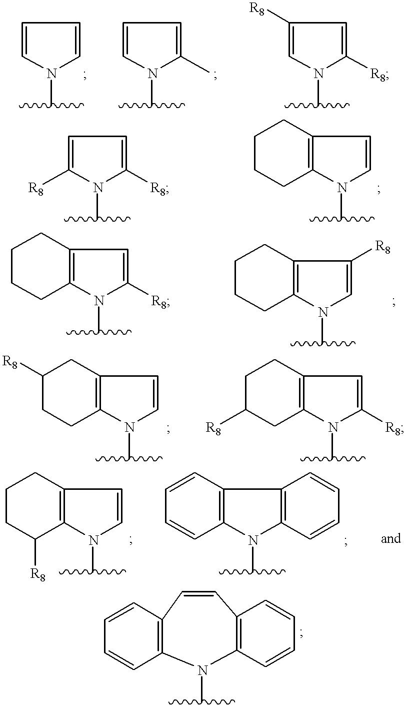
R₇ and R₈ are each independently selected from the group consisting of —-OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

2. The compound of claim 1, wherein each R₁ is 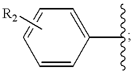
; and
R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C₈ alkoxy or —SR, and 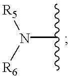
R₅ and R₆ are each independently selected from the group consisting of 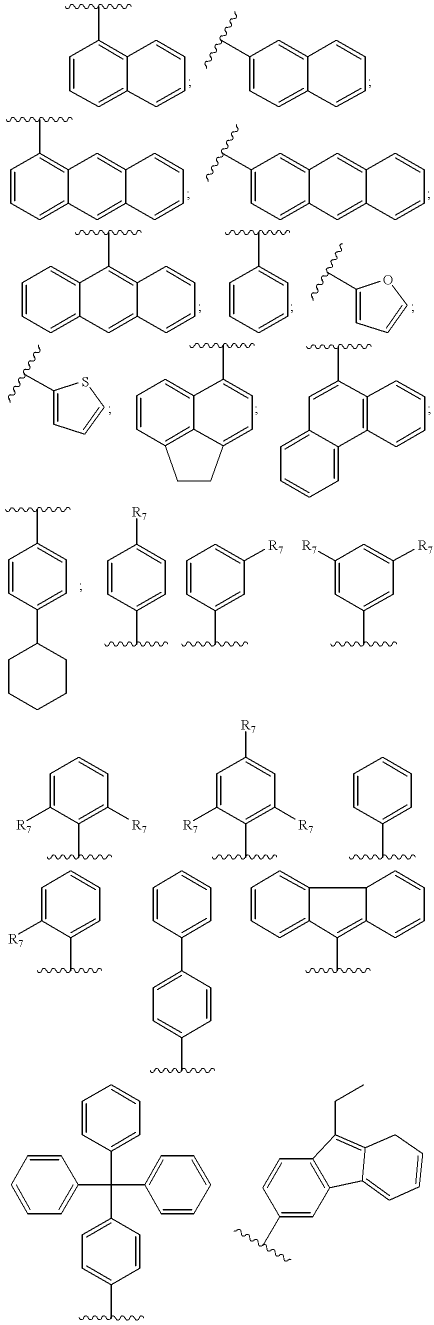
or R₅ and R₆ taken together with the nitrogen atom to which they are attached are independently selected from the group consisting of: 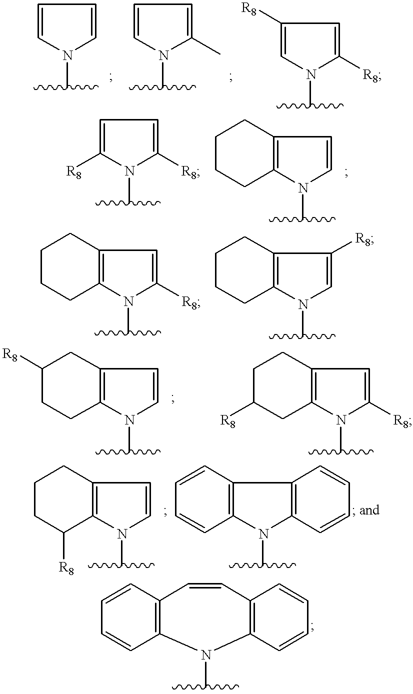
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

3. The compound of claim 2, wherein each R₂ is 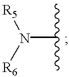
R₅ and R₆ are each independently selected from the group consisting of 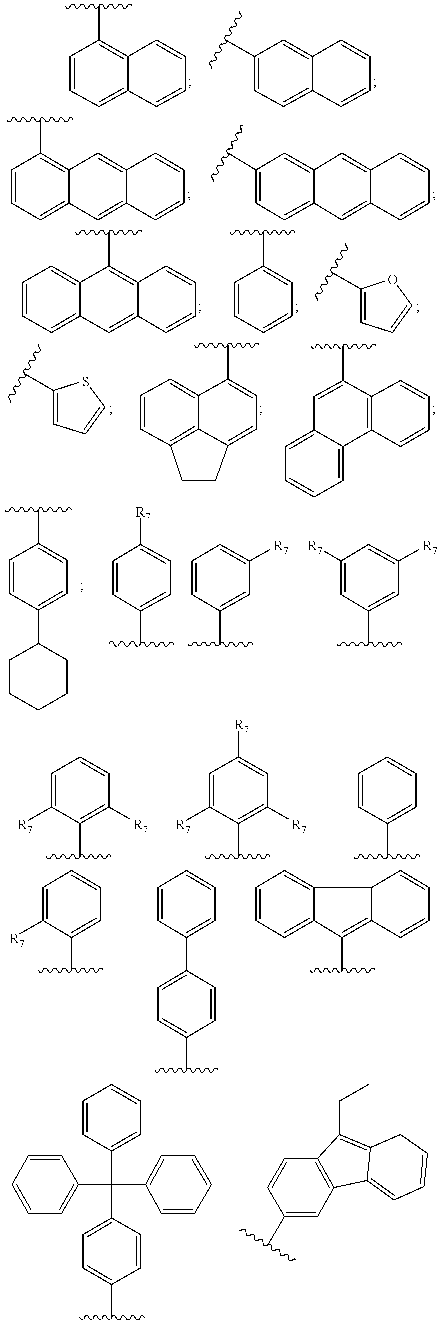
or R₅ and R₆ taken together with the nitrogen atom to which they are attached are independently selected from the group consisting of: 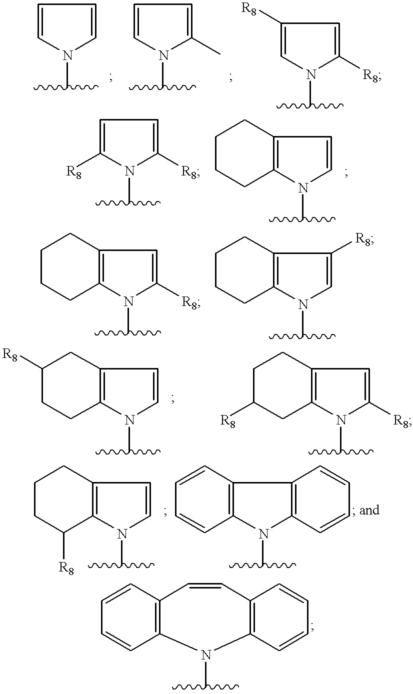
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

4. The compound of claim 3, wherein R₅ and R₆ are each independently selected from the group consisting of 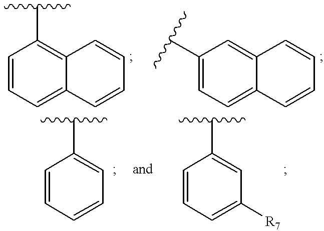
or R₅ and R₆ taken together with the nitrogen atom to which they are attached are independently selected from the group consisting of: 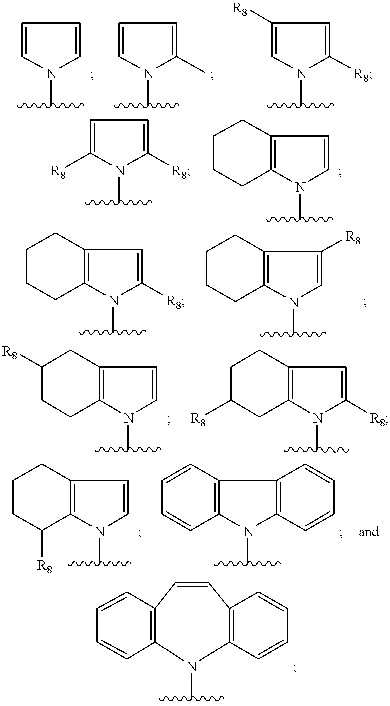
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

5. A hole-injection or hole-transport compound of formula 2: 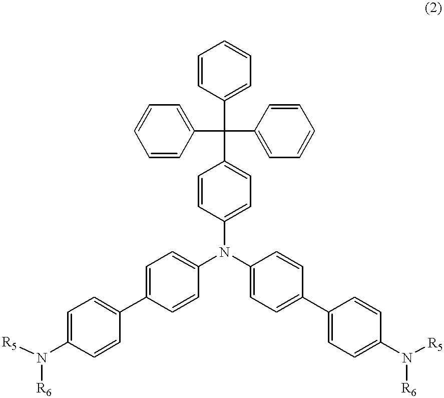
wherein R₅ and R₆ are each independently selected from the group consisting of 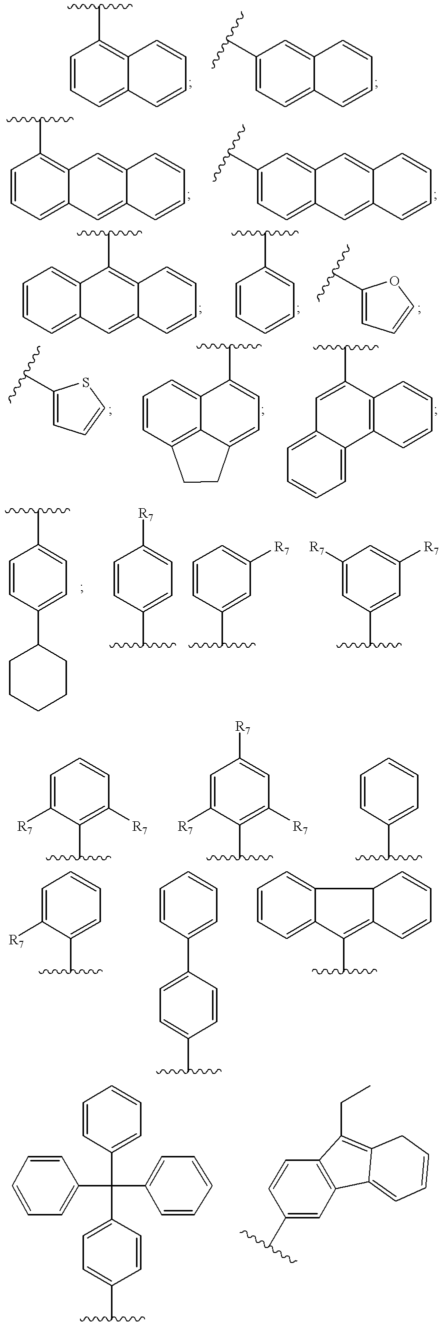
or R₅ and R₆ taken together with the nitrogen atom to which they are attached are independently selected from the group consisting of: 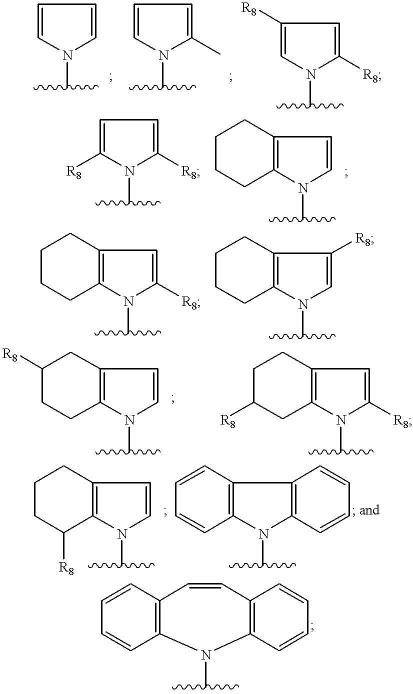
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

6. The compound of claim 5 having the structure:

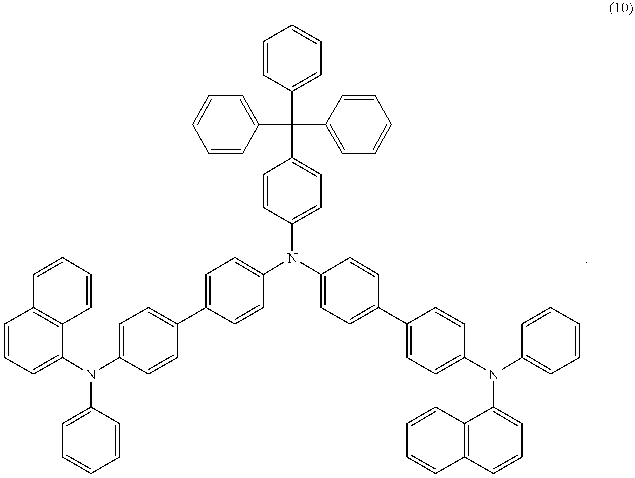

7. The compound of claim 5 having the structure:

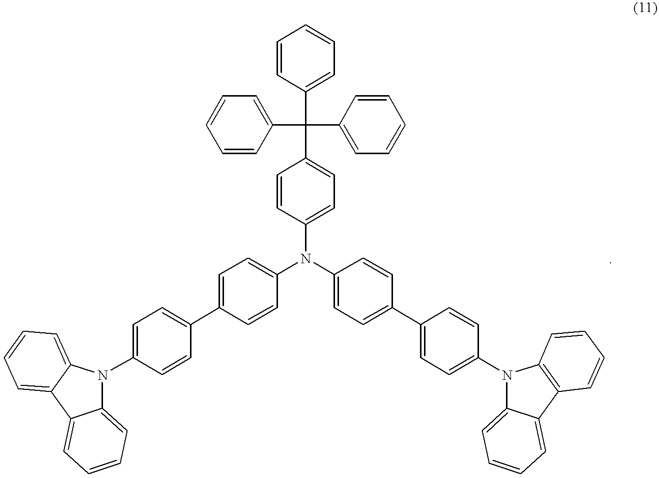

8. The compound of claim 1, wherein each R₁ is 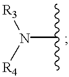
R₃ and R₄ are each independently selected from the group consisting of: 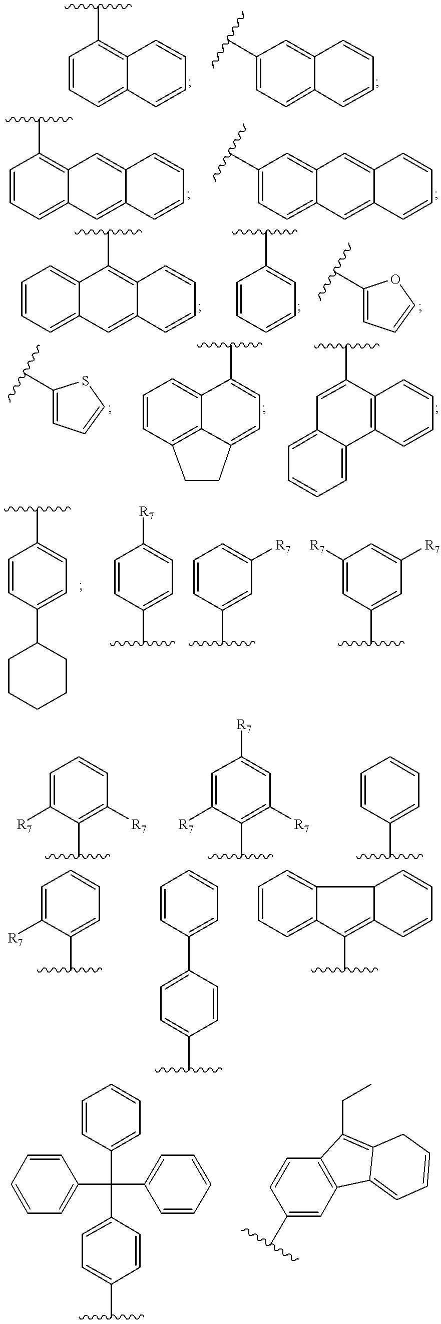
or R₃ and R₄ taken together with the nitrogen atom to which they are attached are independently selected from the group consisting of: 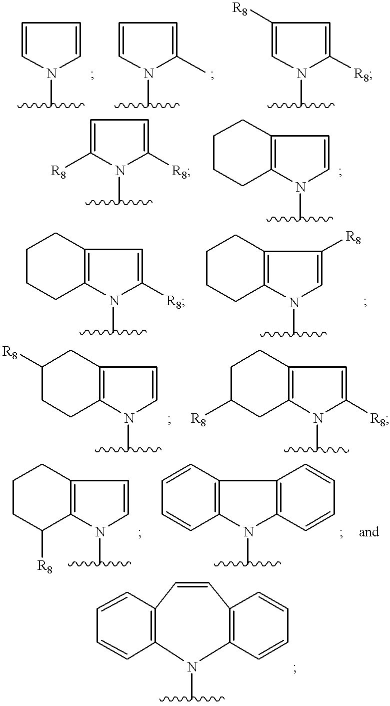
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

9. A hole-injection or hole-transport compound of formula 3: 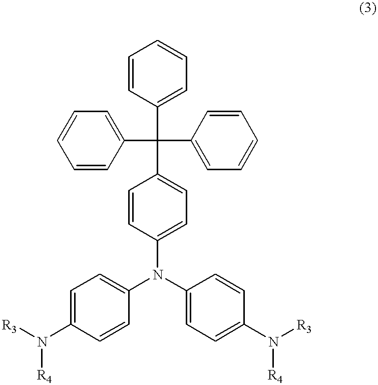
wherein R₃ and R₄ are each independently selected from the group consisting of: 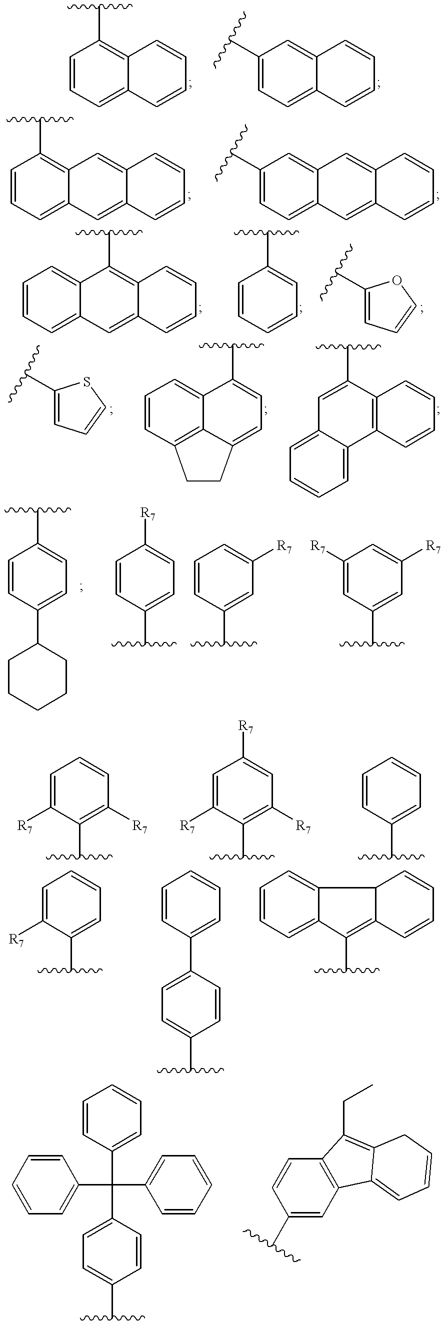
or R₃ and R₄ taken together with the nitrogen atom to which they are attached are independently selected from the group consisting of: 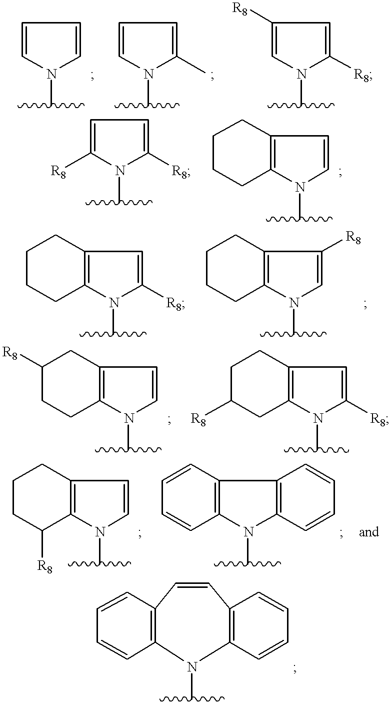
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

10. The compound of claim 1, wherein one of R₁ is: 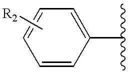
and the other of R₁ is: 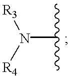
R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C₈ alkoxy or —SR, and 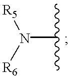
R₃, R_{4,} R₅ and R₆ are each independently selected from the group consisting of: 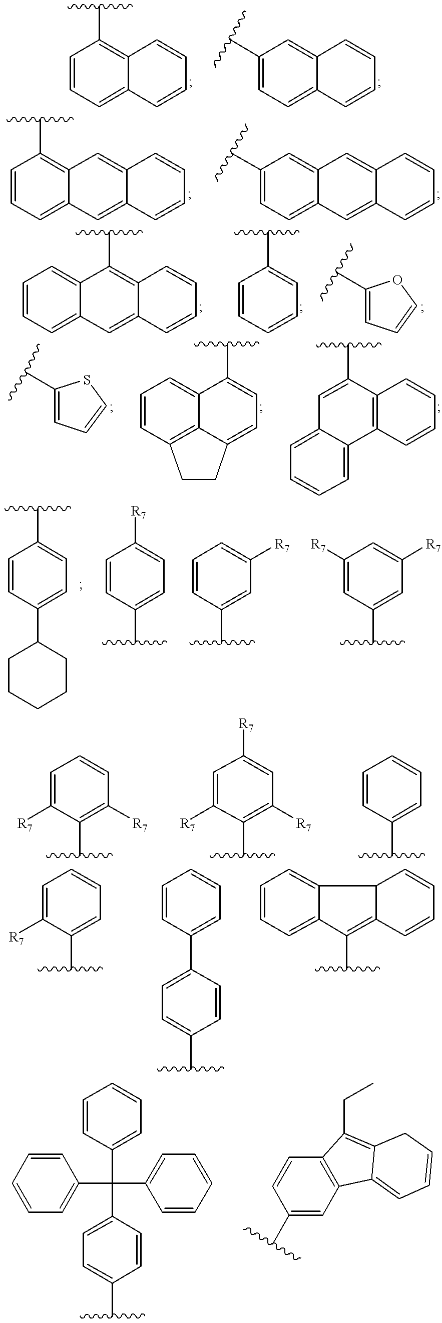
or R₃ and R₄ taken together with the nitrogen atom to which they are attached, or R₅ and R₆ taken together with the nitrogen atom to which they are attached are independently selected from the group consisting of:
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

11. The compound of claim 10, wherein R₂ is 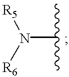
R₅ and R₆ are each independently selected from the group consisting of 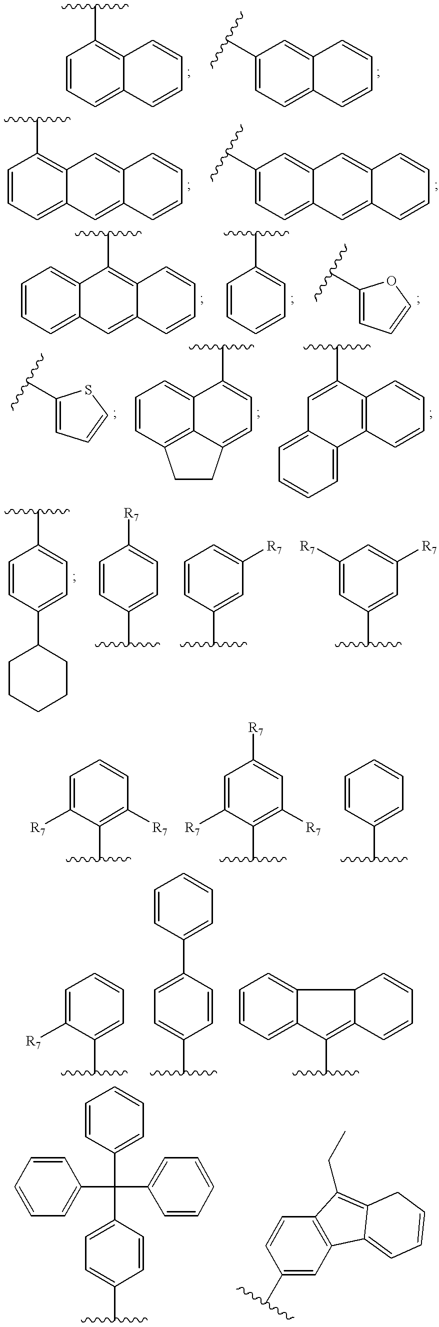
or R₅ and R₆ taken together with the nitrogen atom to which they are attached are independently selected from the group consisting of: 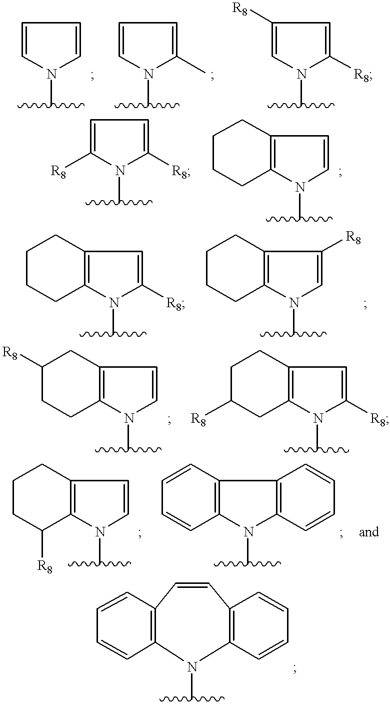
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

12. The compound of claim 11, wherein R₅ and R₆ are each independently selected from the group consisting of 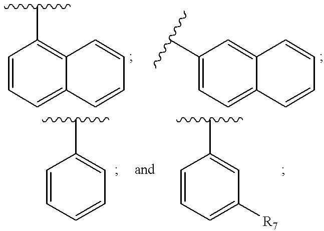
or R₅ and R₆ taken together with the nitrogen atom to which they are attached are selected from the group consisting of: 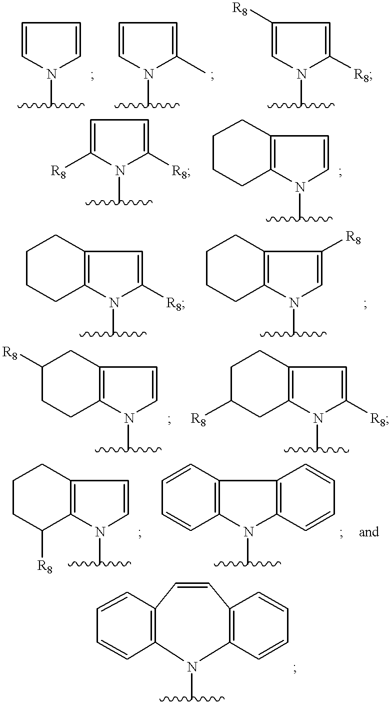
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

13. A hole-injection or hole-transport compound of formula 4: 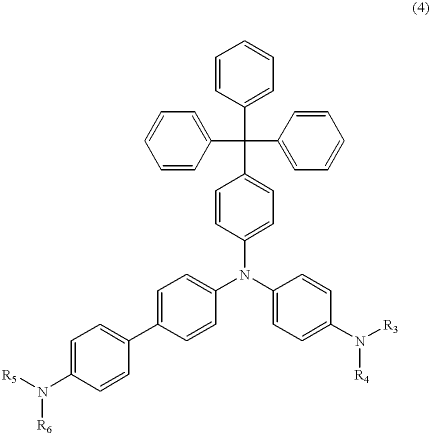
wherein R₃, R_{4,} R₅ and R₆ are each independently selected from the group consisting of 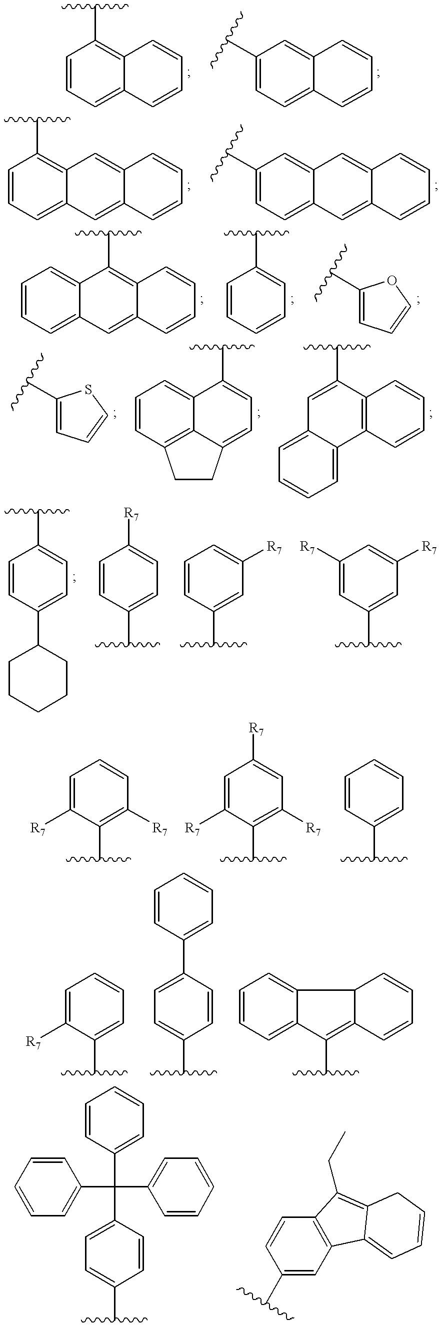
or R₃ and R₄ taken together with the nitrogen to which they are attached or R₅ and R₆ taken together with the nitrogen atom to which they are attached are selected from the group consisting of: 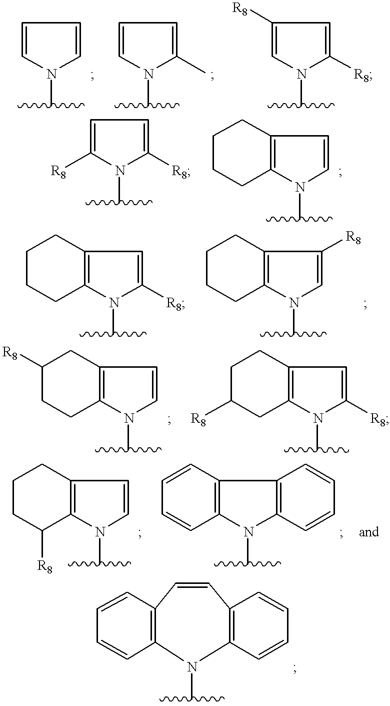
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

14. The compound of claim 13, wherein R₃, R_{4,} R₅ and R₆ are each independently selected from the group consisting of 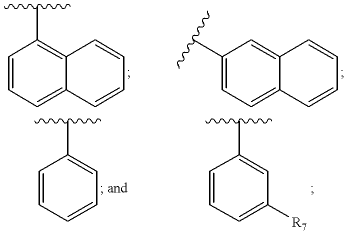
or R₃ and R₄ taken together with the nitrogen atom to which they are attached, or R₅ an d R₆ taken together with the nitrogen atom to which they are attached are independently selected from the group consisting of: 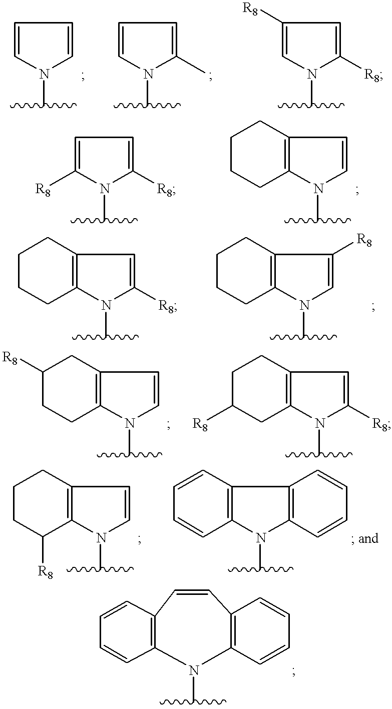
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

15. The compound of claim 1 having the structure: 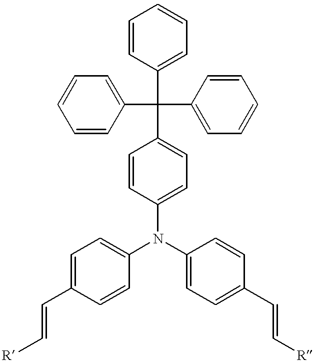
wherein each R′ and R″ is independently selected from the group consisting of hydrogen, C₁-C₆ alkyl, unsubstituted C₆-C₁₈ aryl, C₆-C,₈ aryl that is substituted with C₁-C₆ alkyl, C₁-C₆ alkoxy or C₁-C₆ dialkyl amine, and C₅—C₁₈ aromatic or non-aromatic nitrogen-, oxygen- or sulfur-containing heterocyclic group.

16. An organic light emitting diode device comprising:
(a) a cathode;
(b) an anode; and
(c) at least two organic layers between said anode and said cathode, wherein said at least two organic layers comprise a first organic layer formed from at least one electron-injection/electron-transport material and a second organic layer formed from at least one hole-injection/hole-transport material, wherein said electron-injection/electron-transport material is adjacent to said cathode and said hole-injection/hole-transport material is adjacent to said anode, said at least one hole-injection/hole-transport material comprising a compound of formula 1: 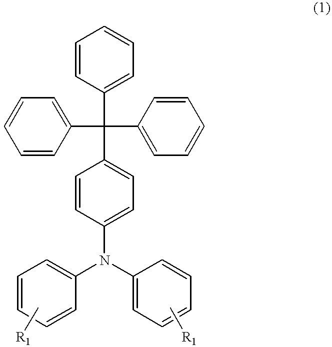
wherein each R₁ is independently selected from the group consisting of hydrogen, C₁-C₆ straight or branched chain alkyl, alkoxy, —CN, —Cl, —F, —CF₃, —SR, —SiR, vinyl that is unsubstituted or substituted at terminal carbon position(s) with C₁-C₆ alkyl or an aromatic group, aryl that is unsubstituted or substituted at para-, meta- or ortho- positions with a C₁-C₆ alkyl, C₁-C₈ alkoxy or —SR, 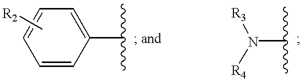
R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C8 alkoxy or —SR, and 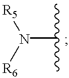
R₃, R_{4,} R₅ and R₆ are each independently selected from the group consisting of 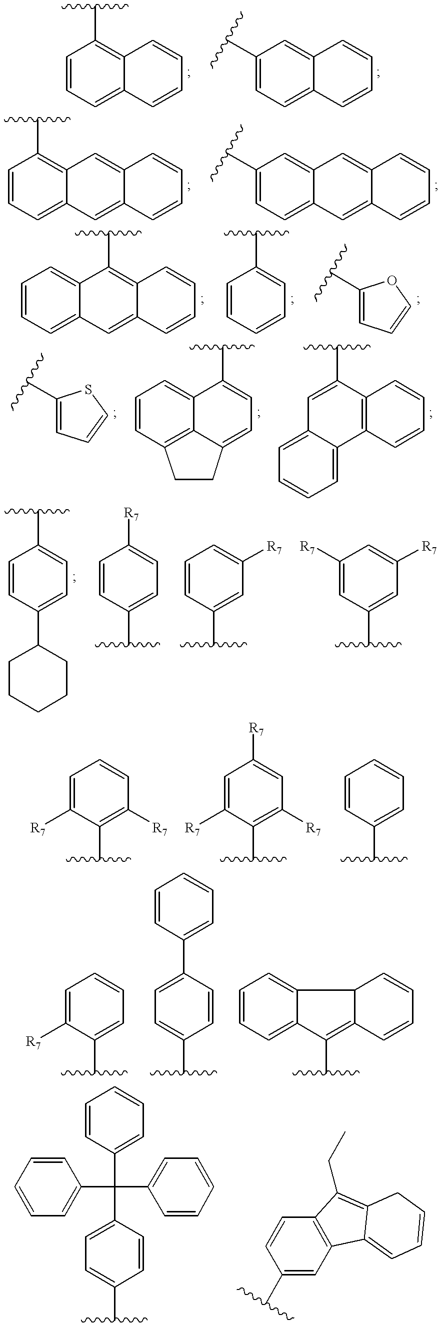
R₃ and R₄ taken together with the nitrogen atom to which they are attached or R₅ and R₆ taken together with the nitrogen atom to which they are attached are independently selected from the group consisting of: 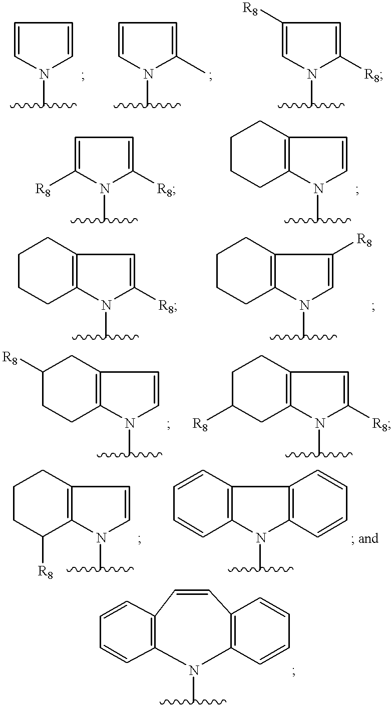
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

17. The device of claim 16, wherein each R₁ is

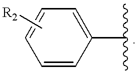

18. The device of claim 17, wherein each R₂ is

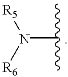

19. The device of claim 16, wherein each R₁ is

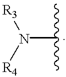

20. The device of claim 16, wherein one of R₁ is: 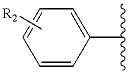
and the other of R₁ is:

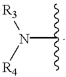

21. The device of claim 16, wherein said anode is a bottom electrode and said cathode is a top electrode.

22. The device of claim 16, wherein said cathode is a bottom electrode and said anode is a top electrode.

23. The device of claim 16, wherein said anode is semi-transparent.

24. The device of claim 16, wherein said cathode is semi-transparent.

25. The device of claim 21, wherein said anode comprises a metal having a high work function, a metal oxide or mixtures thereof.

26. The device of claim 25, wherein said anode comprises a material selected from the group consisting of indium tin oxide, indium zinc tin oxide, indium zinc oxide, ruthenium dioxide, molybdenum oxide, nickel oxide and mixtures thereof.

27. The device of claim 26, wherein said anode comprises indium tin oxide.

28. The device of claim 21, wherein said anode further comprises a layer of dielectric material adjacent to said second organic layer.

29. The device of claim 28, wherein said dielectric material is selected from the group consisting of lithium fluoride, cesium fluoride, silicon oxide and silicon dioxide.

30. The device of claim 21, wherein said anode further comprises a layer of organic conducting material adjacent to said second organic layer.

31. The device of claim 30, wherein said organic conducting material is selected from the group consisting of polyaniline, PEDOT-PSS, and a conducting or semi-conducting organic salt thereof.

32. The device of claim 21, wherein said cathode comprises a material having a low work function.

33. The device of claim 32, wherein the material having a low work function is selected from the group consisting of aluminum, magnesium, calcium, samarium, lithium, cesium, and mixtures thereof.

34. The device of claim 33, wherein said cathode comprises lithium and aluminum.

35. The device of claim 33, wherein said cathode further comprises a layer of dielectric material adjacent to said first organic layer formed from at least one electron-injection/electron-transport material.

36. The device of claim 35, wherein said dielectric material is selected from the group consisting of lithium fluoride, cesium fluoride, lithium chloride and cesium chloride.

37. The device of claim 36, wherein the cathode comprises magnesium and lithium fluoride and further comprises silver.

38. The device of claim 36, wherein the cathode comprises aluminum and lithium fluoride.

39. The device of claim 21, further comprising an emitter layer between said first organic layer and said second organic layer.

40. The device of claim 39, wherein said emitter layer comprises a host compound.

41. The device of claim 40, wherein said host compound is selected from the group consisting of ALQ and IDE-102.

42. The device of claim 39, wherein said emitter layer further comprises a dopant compound.

43. The device of claim 42, wherein said dopant compound is selected from the group consisting of Coumarin 6, Coumarin 485, Coumarin, 487, Coumarin 490, Coumarin 498, Coumarin 500, Coumarin 503, Coumarin 504, Coumarin 504T, Coumarin 510, Coumarin 515, Coumarin 519, Coumarin 521, Coumarin 521T, Coumarin 522B, Coumarin 523, Coumarin 525, Coumarin 535, Coumarin 540A, Coumarin 545, a quinacridone derivative, a distyrylamine derivative, IDE-102, rubrene, DCJTB, pyrromethane 546, and mixtures thereof.

44. The device of claim 21, wherein said at least one electron-injection/electron-transport material comprises a compound selected from the group consisting of ALQ, and an oxadiazole derivative.

45. The device of claim 44, wherein said at least one electron-injection/electron-transport material is ALQ.

46. The device of claim 21, wherein said device is a microdisplay device.

47. An organic light emitting diode device comprising:
(a) a cathode;
(b) an anode;
(c) a layer formed from at least one electron-injection/electron-transport material that is adjacent to said cathode;
(d) a hole-injection layer that is adjacent to said anode; and
(e) at least one hole-transport layer that is adjacent to said hole-injection layer, wherein at least one of said hole-injection and hole-transport layers comprises a compound of formula 1: 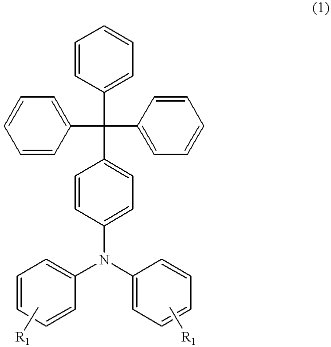
wherein each R₁ is independently selected from the group consisting of hydrogen, C₁-C₆ straight or branched chain alkyl, alkoxy, —CN, —Cl, —F, —CF₃, —SR, —SiR, vinyl that is unsubstituted or substituted at terminal carbon position(s) with C₁-C₆ alkyl or an aromatic group, aryl that is unsubstituted or substituted at para-, meta- or ortho- positions with a C₁-C₆ alkyl, C₁-C₈ alkoxy or —SR, 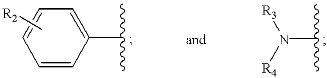
R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C₈ alkoxy or —SR, and 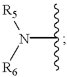
R₃, R₄, R₅ and R₆ are each independently selected from the group consisting of 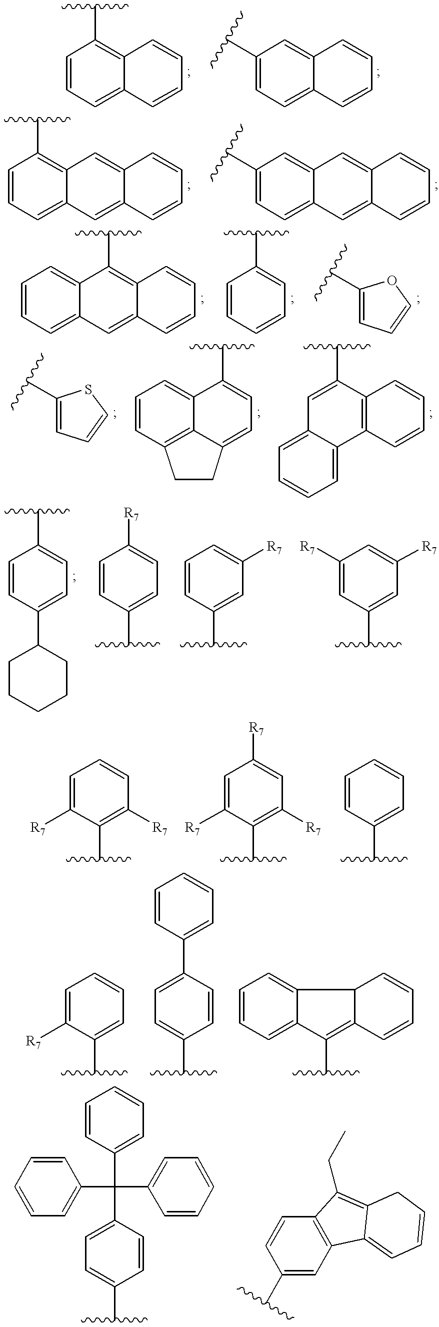
or R₃ and R₄ taken together with the nitrogen atom to which they are attached or R₅ and R₆ taken together with the nitrogen atom to which they are attached are independently selected from the group consisting of: 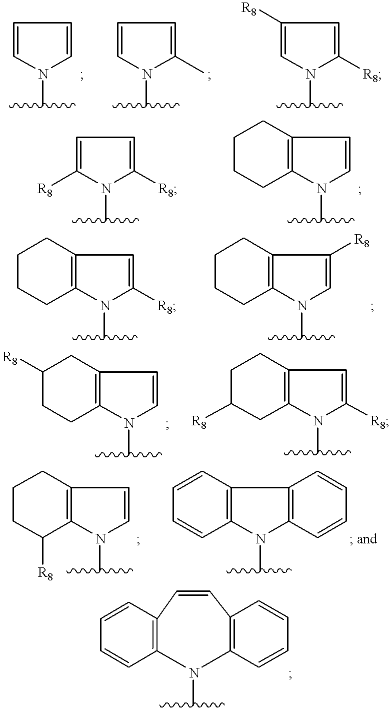
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

48. The device of claim 47, wherein each R₁ is

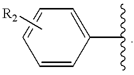

49. The device of claim 48, wherein each R₂ is

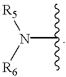

50. The device of claim 47, wherein each R₁ is

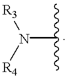

51. The device of claim 47, wherein one of R₁ is: 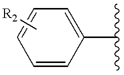
and the other of R₁ is:

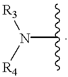

52. The device of claim 47, wherein said anode is a bottom electrode and said cathode is a top electrode.

53. The device of claim 47, wherein said cathode is a bottom electrode and said anode is a top electrode.

54. The device of claim 47, wherein said anode is semi-transparent.

55. The device of claim 47, wherein said cathode is semi-transparent.

56. The device of claim 52, wherein said anode comprises a metal having a high work function, a metal oxide or mixtures thereof.

57. The device of claim 56, wherein said anode comprises a material selected from the group consisting of indium tin oxide, indium zinc tin oxide, indium zinc oxide, ruthenium dioxide, molybdenum oxide, nickel oxide and mixtures thereof.

58. The device of claim 57, wherein said anode comprises indium tin oxide.

59. The device of claim 52, wherein said anode further comprises a layer of dielectric material adjacent to said second hole-injection layer.

60. The device of claim 59, wherein said dielectric material is selected from the group consisting of lithium fluoride, cesium fluoride, silicon oxide and silicon dioxide.

61. The device of claim 52, wherein said anode further comprises a layer of organic conducting material adjacent to said hole-injection layer.

62. The device of claim 61, wherein said organic conducting material is selected from the group consisting of polyaniline, PEDOT-PSS, and a conducting or semi-conducting organic salt thereof.

63. The device of claim 52, wherein said cathode comprises a material having a low work function.

64. The device of claim 63, wherein the material having a low work function is selected from the group consisting of aluminum, magnesium, calcium, samarium, lithium, cesium, and mixtures thereof.

65. The device of claim 64, wherein said cathode comprises lithium and aluminum.

66. The device of claim 52, wherein said cathode further comprises a layer of dielectric material adjacent to said layer formed from at least one electron-injection/electron-transport material.

67. The device of claim 66, wherein said dielectric material is selected from the group consisting of lithium fluoride, cesium fluoride, lithium chloride and cesium chloride.

68. The device of claim 67, wherein the cathode comprises magnesium and lithium fluoride and further comprises silver.

69. The device of claim 67, wherein the cathode comprises aluminum and lithium fluoride.

70. The device of claim 52, further comprising an emitter layer between said organic layer formed from at least one electron-injection/electron-transport material and said at least one hole-transport layer.

71. The device of claim 70, wherein said emitter layer comprises a host compound.

72. The device of claim 71, wherein said host compound is selected from the group consisting of ALQ and IDE-102.

73. The device of claim 71, wherein said emitter layer further comprises a dopant compound.

74. The device of claim 73, wherein said dopant compound is selected from the group consisting of Coumarin 6, Coumarin 485, Coumarin, 487, Coumarin 490, Coumarin 498, Coumarin 500, Coumarin 503, Coumarin 504, Coumarin 504T, Coumarin 510, Coumarin 515, Coumarin 519, Coumarin 521, Coumarin 521T, Coumarin 522B, Coumarin 523, Coumarin 525, Coumarin 535, Coumarin 540A, Coumarin 545, a quinacridone derivative, a distyrylamine derivative, IDE-102, rubrene, DCJTB, pyrromethane 546, and mixtures thereof.

75. The device of claim 52, wherein said at least one electron-injection/electron-transport material comprises a compound selected from the group consisting of ALQ, and an oxadiazole derivative.

76. The device of claim 75, wherein said at least one electron-injection/electron-transport material is ALQ.

77. The device of claim 52, further comprising a first hole-transport layer and a second hole-transport layer.

78. The device of claim 77, wherein at least two of the first hole-transport layer, the second hole-transport layer and the hole-injection layer are inter-deposited.

79. The device of claim 52, wherein said device is a microdisplay device.

80. A microdisplay device, comprising:
(a) at least one bottom electrode that is an anode;
(b) at least one top electrode that is a cathode; and
(c) at least two organic layers between said at least one bottom electrode and said at least one top electrode, wherein said at least two organic layers comprise a first organic layer formed from at least one electron-injection/electron-transport material that is adjacent to said at least one cathode and a second organic layer formed from at least one hole-injection/hole-transport material that is adjacent to said at least one anode, said at least one hole-injection/hole-transport material comprising a compound of formula 1: 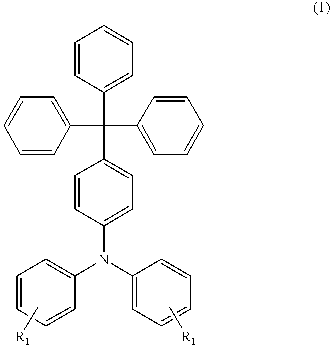
wherein each R₁ is independently selected from the group consisting of hydrogen, C₁-C₆ straight or branched chain alkyl, alkoxy, —CN, —Cl, —F, —CF₃, —SR, —SiR, vinyl that is unsubstituted or substituted at terminal carbon position(s) with C₁-C₆ alkyl or an aromatic group, aryl that is unsubstituted or substituted at para-, meta- or ortho- positions with a C₁-C₆ alkyl, C₁-C₈ alkoxy or —SR, 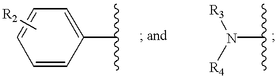
R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C₈ alkoxy or —SR, and 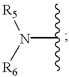
R₃, R₄, R₅ and R₆ are each independently selected from the group consisting of 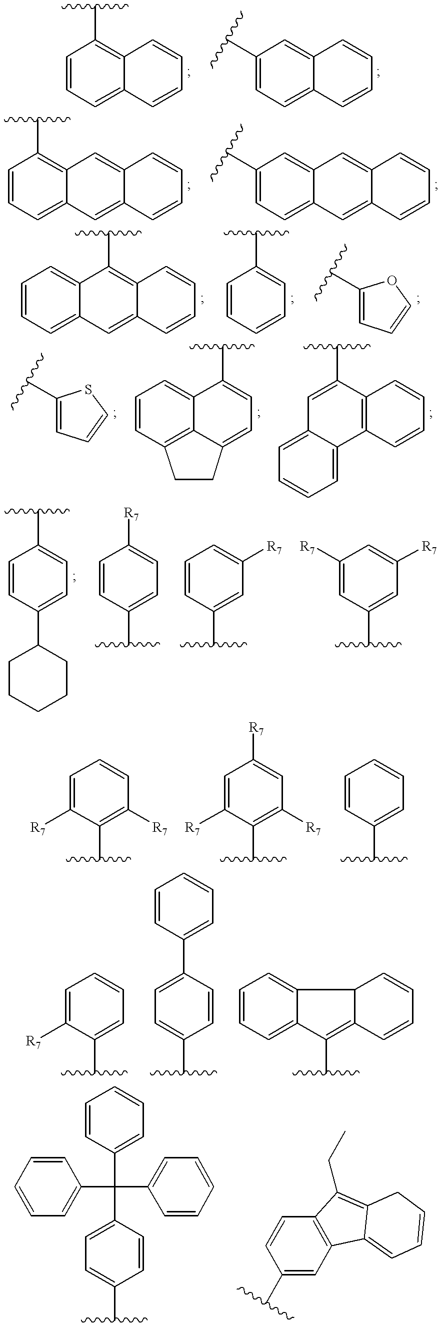
or R₃ and R₄ taken together with the nitrogen atom to which they are attached or R₅ and R₆ taken together with the nitrogen atom to which they are attached are independently selected from the group consisting of: 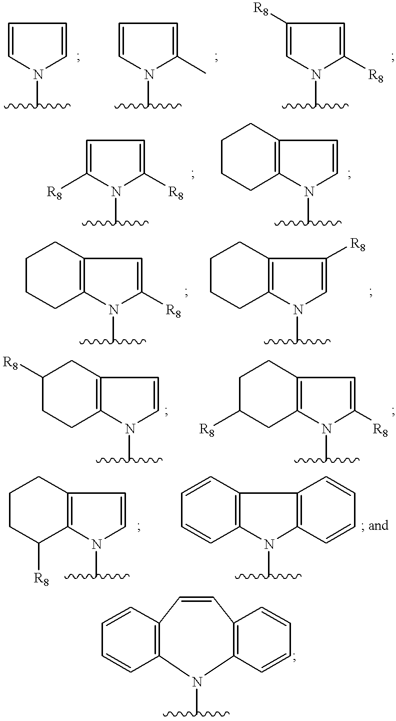
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

81. The device of claim 80, wherein each R₁ is

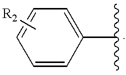

82. The device of claim 81, wherein each R₂ is

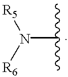

83. The device of claim 82, wherein each R₅ and R₆ is independently selected from the group consisting of: 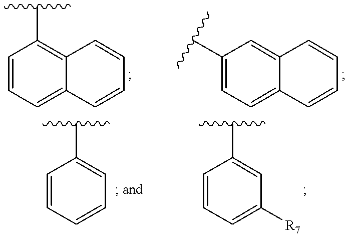
or R₅ and R₆ taken together with the nitrogen atom to which they are attached are selected from the group consisting of: 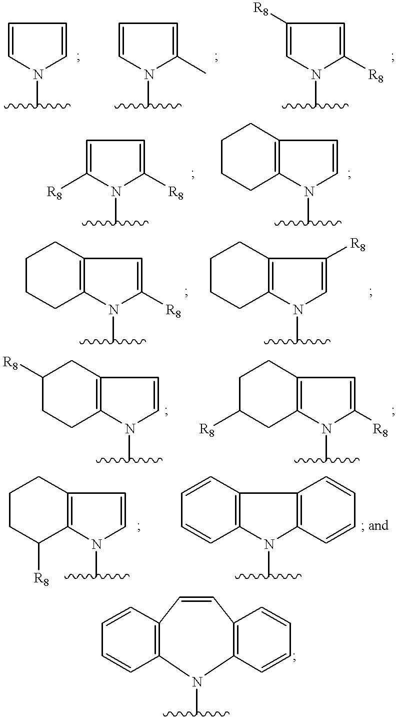
R₇ and R₉ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

84. The device of claim 80, wherein each R₁ is

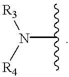

85. The device of claim 80, wherein one of R₁ is: 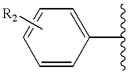
and the other of R₁ is:

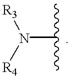

86. An organic light emitting diode device comprising:
(a) a cathode;
(b) an anode; and
(c) at least two organic layers between said anode and said cathode, wherein said at least two organic layers comprise a first organic layer formed from at least one electron-injection/electron-transport material and a second organic layer formed from at least one hole-injection/hole-transport material, wherein said electron-injection/electron-transport material is adjacent to said cathode and said hole-injection/hole-transport material is adjacent to said anode, said at least one hole-injection/hole-transport material comprising a compound of formula 2: 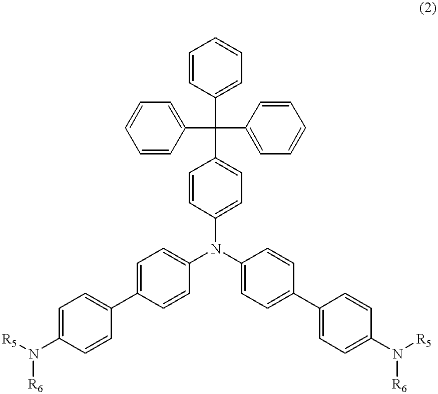
wherein R₅ and R₆ are each independently selected from the group consisting of 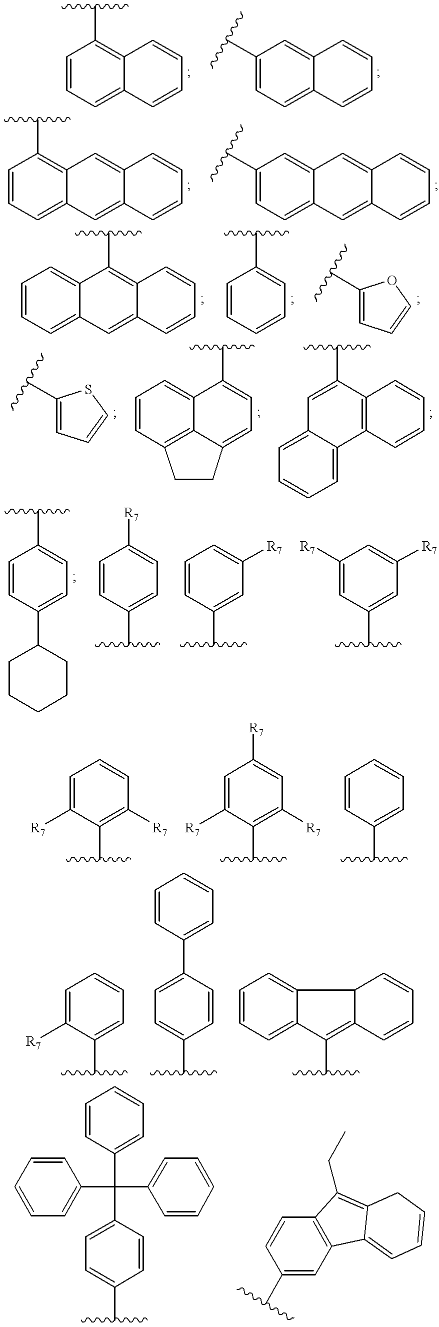
or R₅ and R₆ taken together with the nitrogen atom to which they are attached are independently selected from the group consisting of: 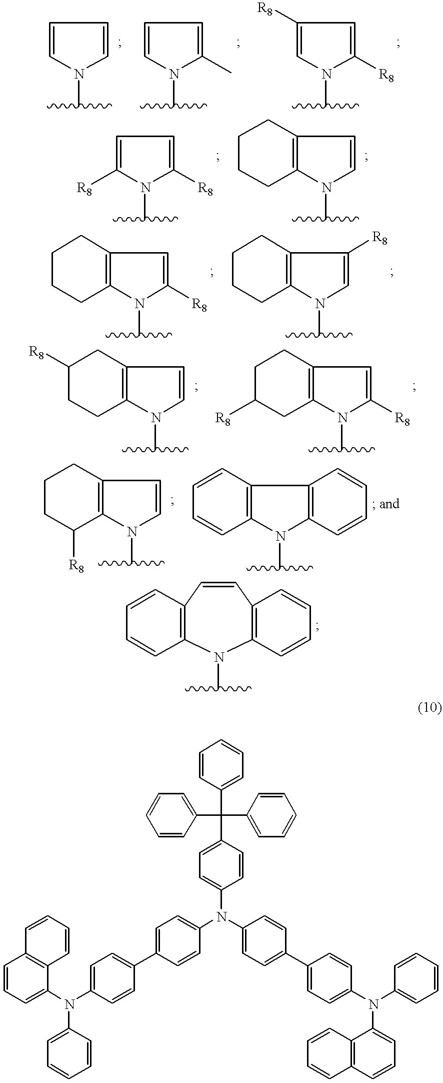
R₇ and R₈ are each independently selected from the group consisting of —OR₉, C₁-C₄ alkyl, aryl, —SCH₃, —CF₃, —Cl, —Br, —NO₂, —SR, —SiR and COOR₉;
R is C₁-C₆ straight or branched chain alkyl; and
R₉ is selected from the group consisting of C₁-C₆ alkyl and aryl.

87. The device of claim 86, wherein said at least one hole-injection/hole-transport material comprises the compound.

88. The device of claim 86, wherein said at least one hole-injection/hole-transport material comprises the compound

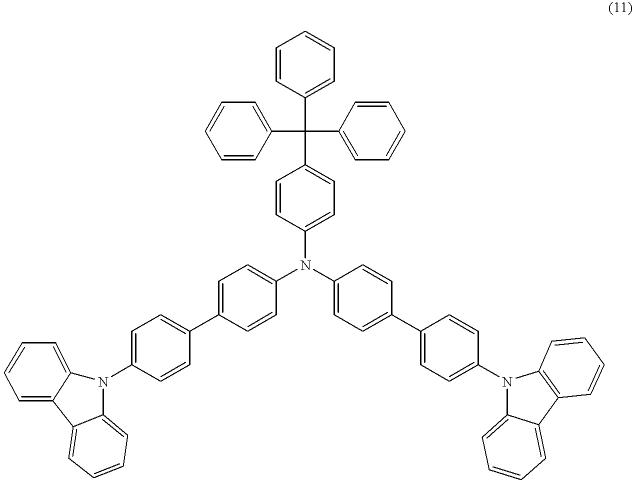

89. The device of claim 86, wherein said at least one hole-injection/hole-transport material comprises the compound 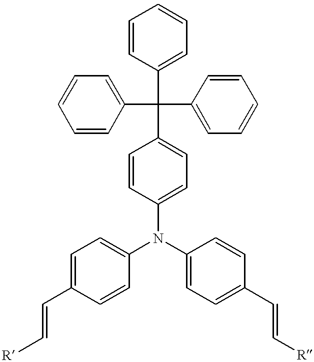
wherein each R′ and R″ is independently selected from the group consisting of hydrogen, C₁-C₆ alkyl, unsubstituted C₆-C₁₈ aryl, C₆-C₁₈ aryl that is substituted with C₁-C₆ alkyl, C₁-C₆ alkoxy or C₁-C₆ dialkyl amine, and C₅-C₁₈ aromatic or non-aromatic nitrogen-, oxygen- or sulfur-containing heterocyclic group.

90. An organic light emitting diode device comprising:
(a) a cathode;
(b) an anode; and
(c) at least two organic layers between said anode and said cathode, wherein said at least two organic layers comprise a first organic layer formed from at least one electron-injection/electron-transport material and a second organic layer formed from at least one hole-injection/hole-transport material, wherein said electron-injection/electron-transport material is adjacent to said cathode and said hole-injection/hole-transport material is adjacent to said anode, said at least one hole-injection/hole-transport material comprising a compound of formula 3: 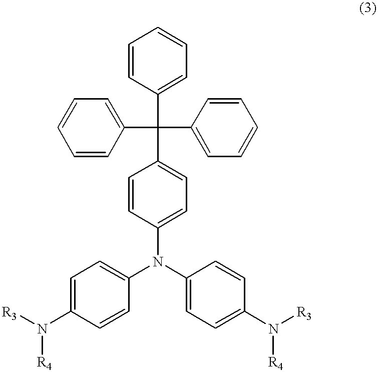
wherein R₃ and R₄ are each independently selected from the group consisting of: 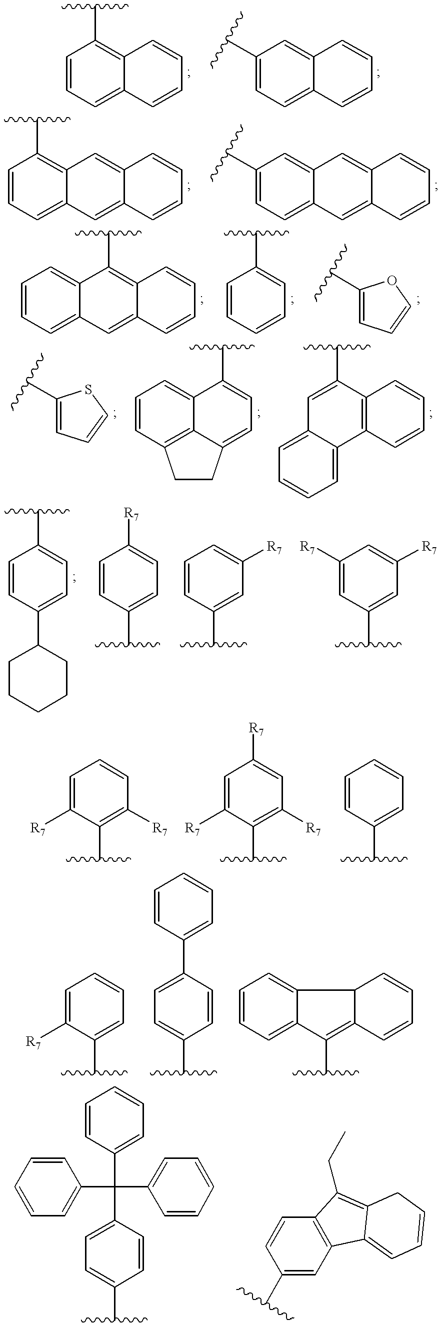
or R₃ and R₄ taken together with the nitrogen atom to which they are attached are independently selected from the group consisting of: 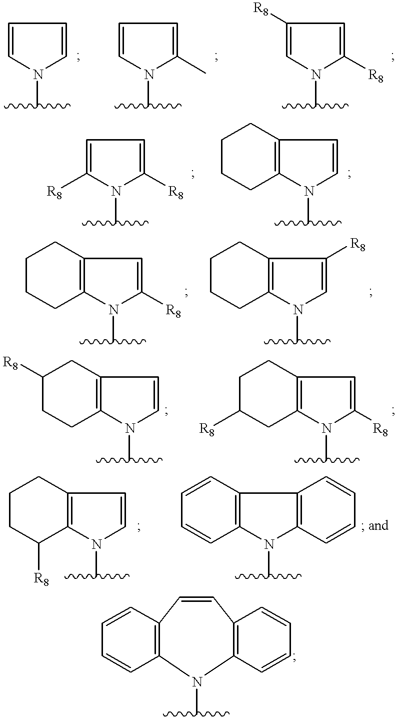
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

91. An organic light emitting diode device comprising:
(a) a cathode;
(b) an anode; and
(c) at least two organic layers between said anode and said cathode, wherein said at least two organic layers comprise a first organic layer formed from at least one electron-injection/electron-transport material and a second organic layer formed from at least one hole-injection/hole-transport material, wherein said electron-injection/electron-transport material is adjacent to said cathode and said hole-injection/hole-transport material is adjacent to said anode, said at least one hole-injection/hole-transport material comprising a compound of formula 4: 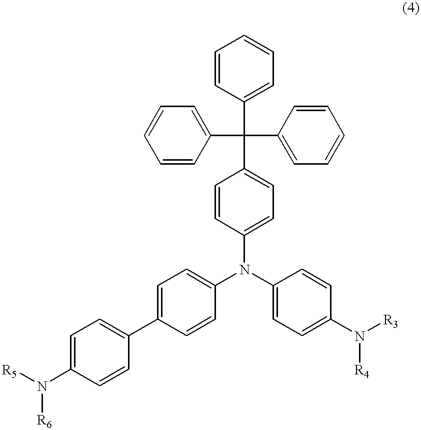
wherein R₃, R₄, R₅ and R₆ are each independently selected from the group consisting of 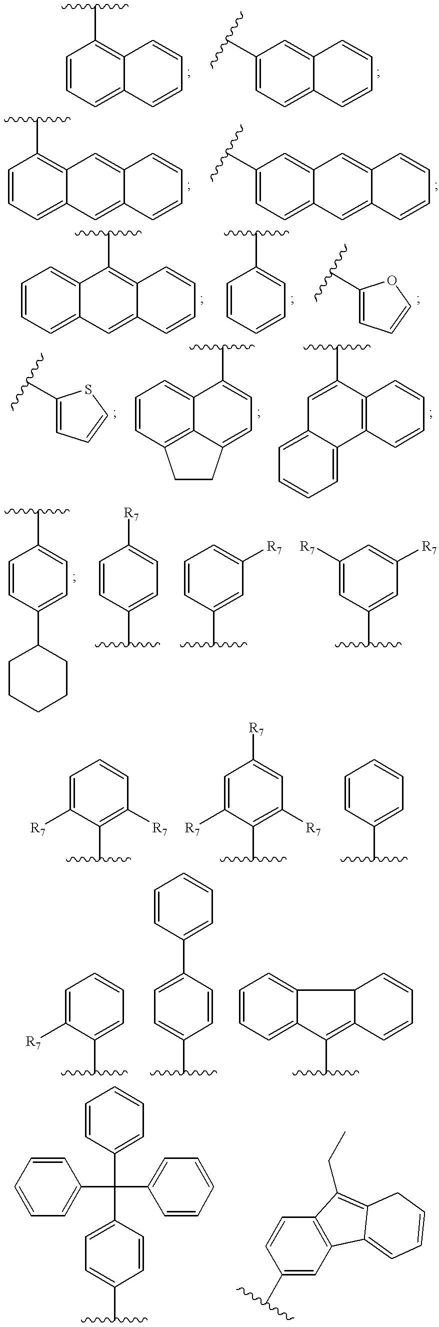
or R₃ and R₄ taken together with the nitrogen atom to which they are attached or R₅ and R₆ taken together with the nitrogen atom to which they are attached are independently selected from the group consisting of: 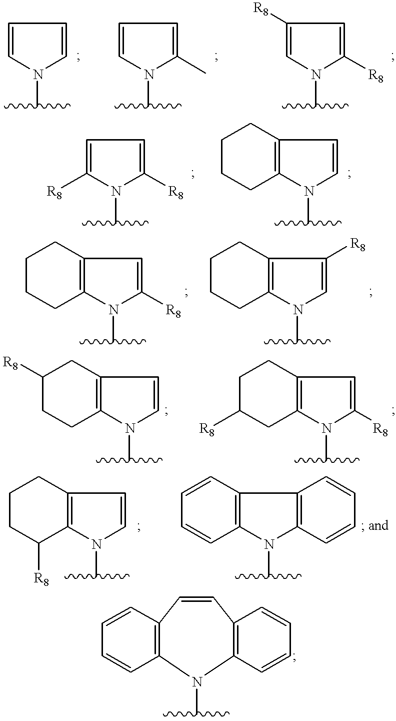
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

92. The device of claim 91, wherein R₃, R₄, R₅ and R₆ are each independently selected from the group consisting of 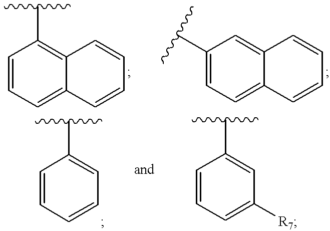
or R₃ and R₄ taken together with the nitrogen atom to which they are attached or R₅ and R₆ taken together with the nitrogen atom to which they are attached are independently selected from the group consisting of: 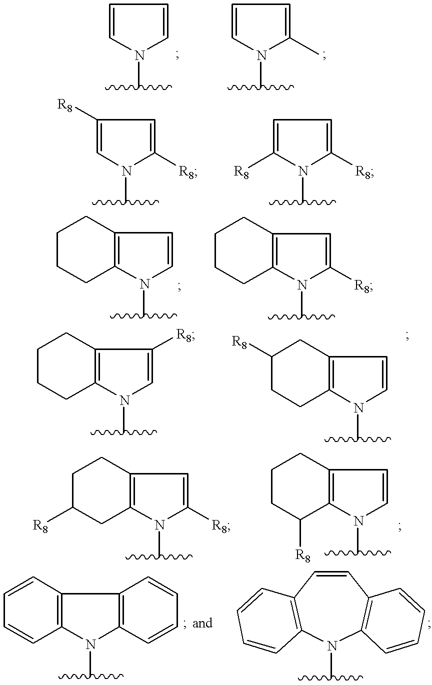
R₇ and R₈ are each independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

93. The compound of claim 1, wherein R₃, R₄, R₅ and R₆ are each independently selected from the group consisting of: 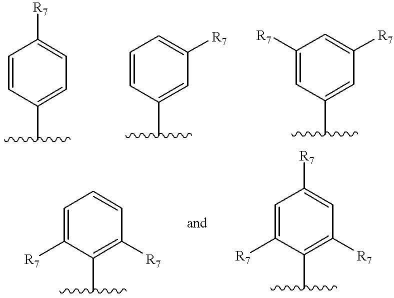
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

94. The compound of claim 1, wherein R₃, R₄, R₅ and R₆ are each independently selected from the group consisting of: 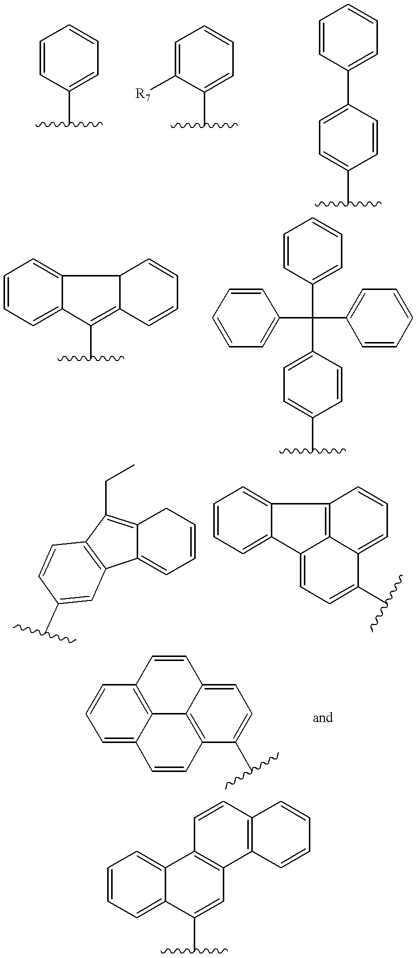
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

95. The compound of claim 5, wherein R₅ and R₆ are each independently selected from the group consisting of: 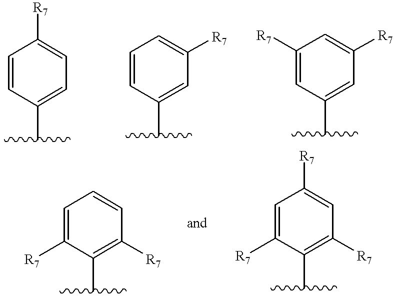
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

96. The compound of claim 5, wherein R₅ and R₆ are each independently selected from the group consisting of: 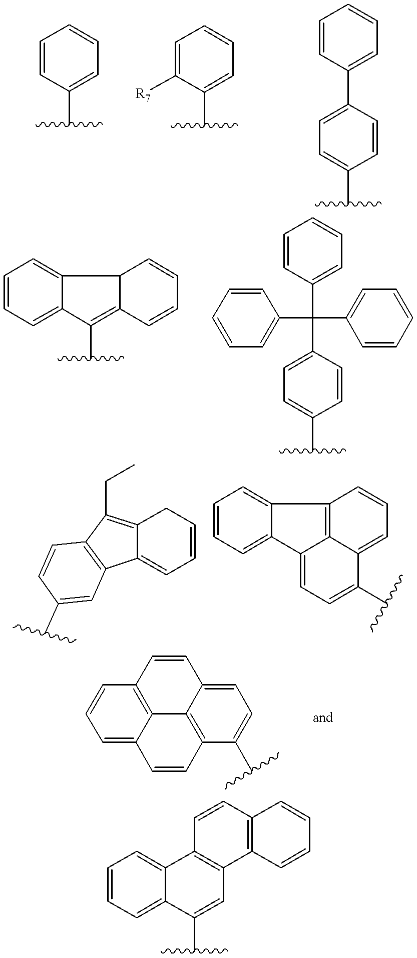
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

97. The compound of claim 9, wherein R₃ and R₄ are each independently selected from the group consisting of: 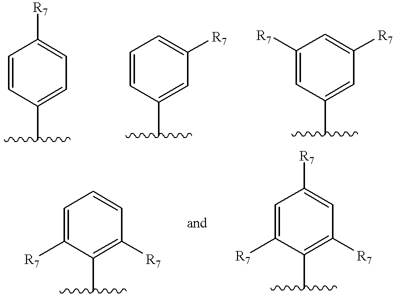
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

98. The compound of claim 9, wherein R₃ and R₄ are each independently selected from the group consisting of: 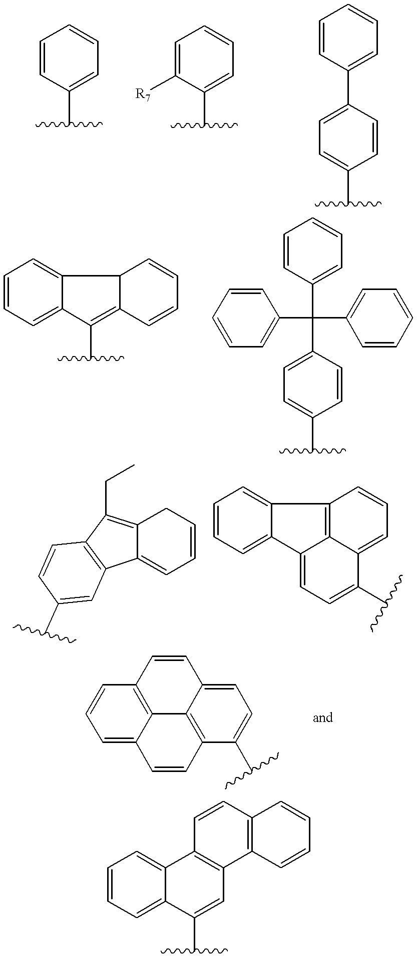
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

99. The compound of claim 13, wherein R₃, R_{4,} R₅ and R₆ are each independently selected from the group consisting of: 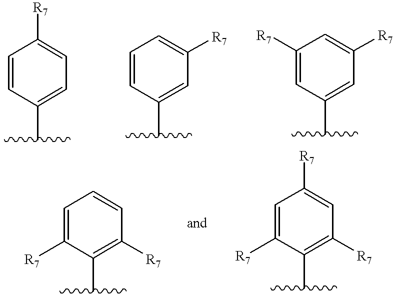
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

100. The compound of claim 13, wherein R₃, R₄, R₅ and R₆ are each independently selected from the group consisting of: 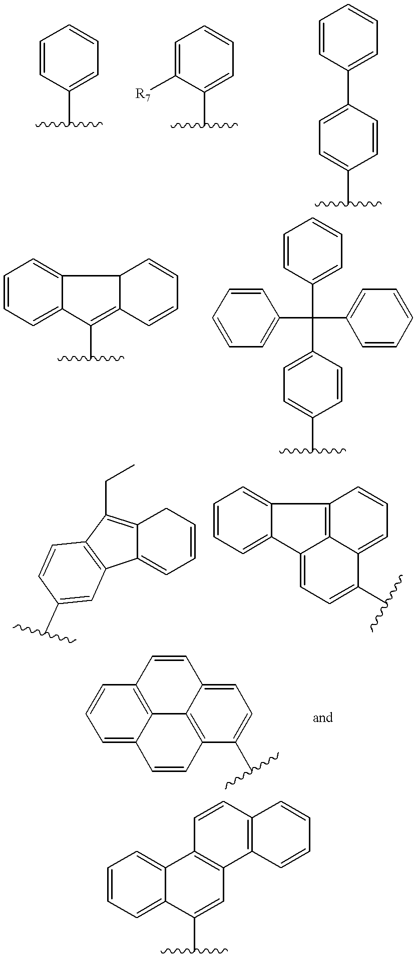
wherein R₇ is selected from the group consisting of —OR9, C₁-C₄ alkyl, aryl, —SCH₃, —CF₃, —C₁, —Br, —NO₂, —SR, —SiR and —COOR₉;
R is C₁-C₆ straight or branched chain alkyl; and
R₉ is selected from the group consisting of C₁-C₆ alkyl and aryl.

101. The device of claim 16, wherein R₃, R₄, R₅ and R₆ are each independently selected from the group consisting of: 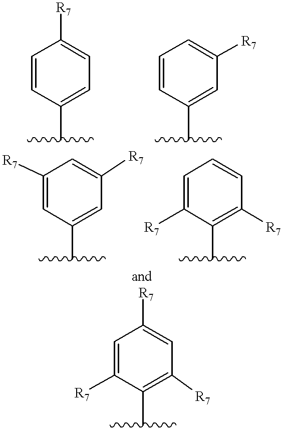
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

102. The device of claim 16, wherein R₃, R₄, R₅ and R₆ are each independently selected from the group consisting of: 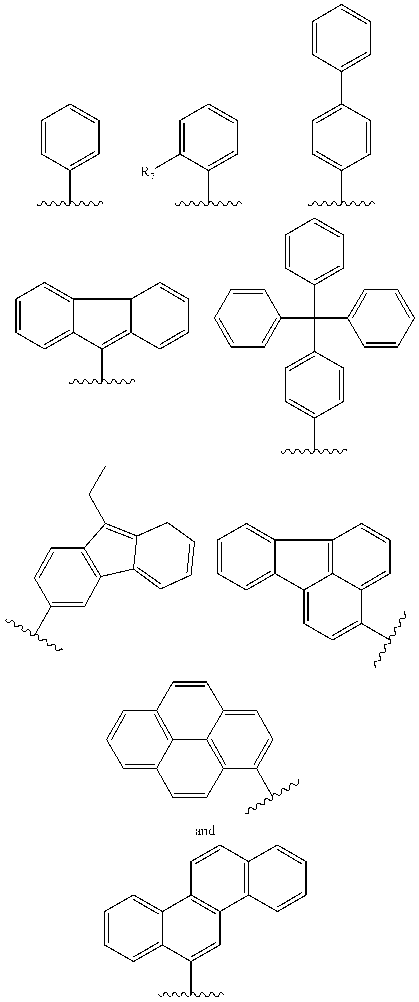
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

103. The device of claim 47, wherein R₃, R₄, R₅ and R₆ are each independently selected from the group consisting of: 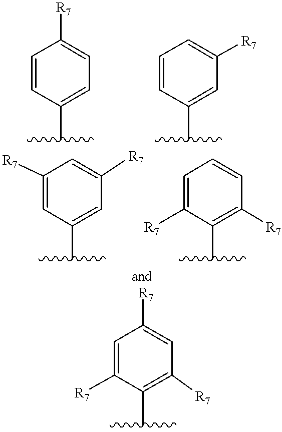
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

104. The device of claim 47, wherein R₃, R₄, R₅ and R₆ are each independently selected from the group consisting of: 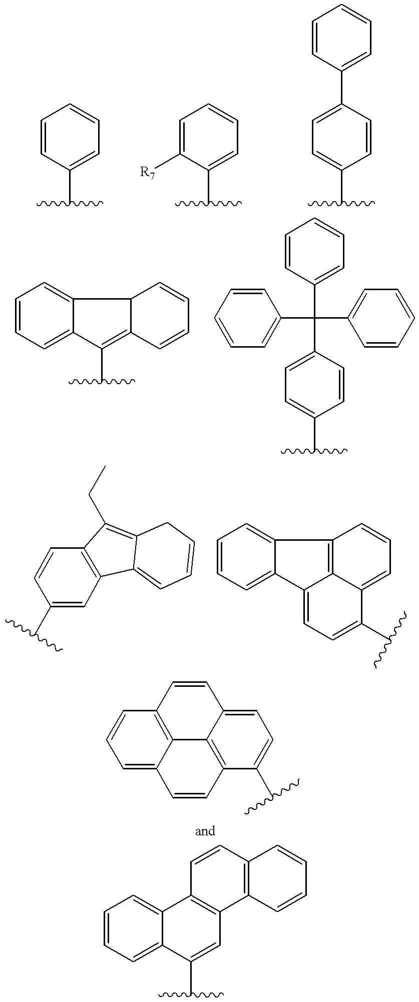
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

105. The device of claim 80, wherein R₃, R_{4,} R₅ and R₆ are each independently selected from the group consisting of: 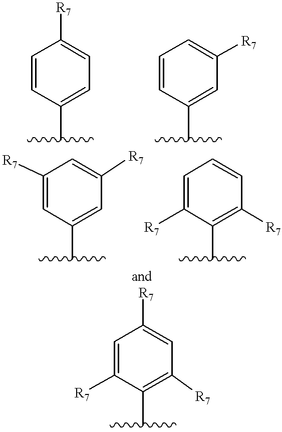
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

106. The device of claim 80, wherein R₃, R_{4,} R₅ and R₆ are each independently selected from the group consisting of: 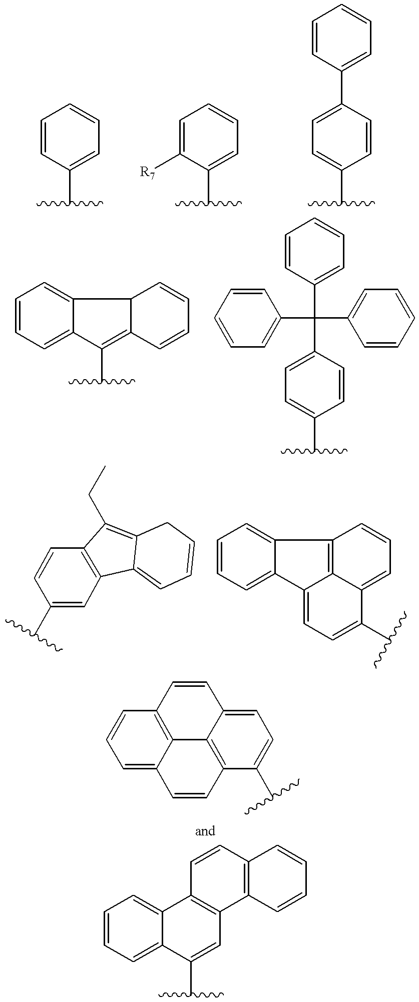
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

107. The device of claim 86, wherein R₅ and R₆ are each independently selected from the group consisting of: 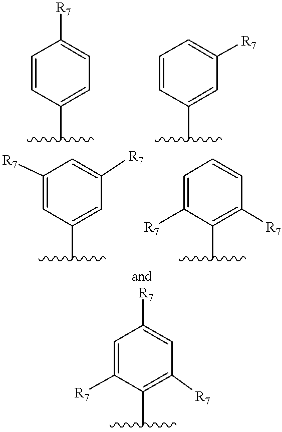
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

108. The device of claim 86, wherein R₅ and R₆ are each independently selected from the group consisting of: 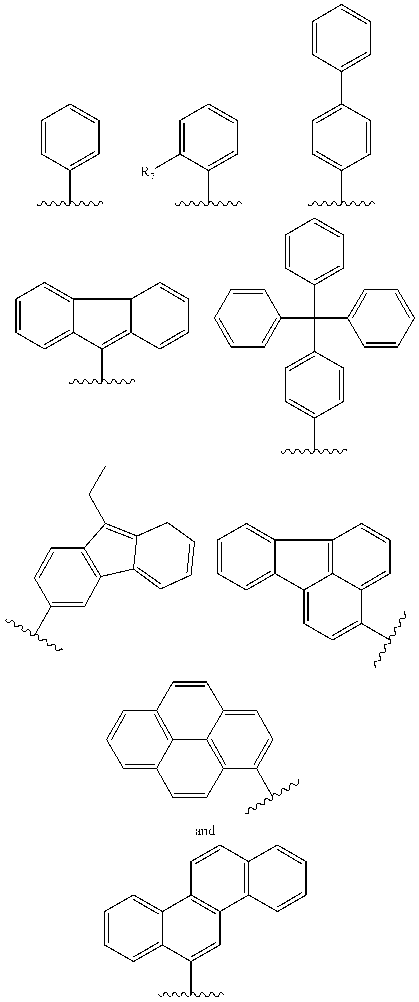
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

109. The device of claim 90, wherein R₃ and R₄ are each independently selected from the group consisting of: 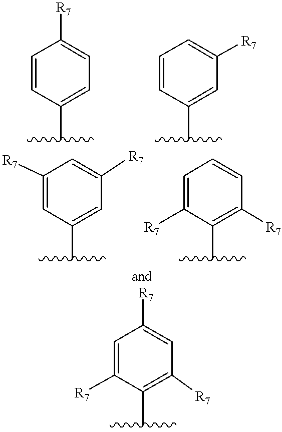
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

110. The device of claim 90, wherein R₃ and R₄ are each independently selected from the group consisting of: 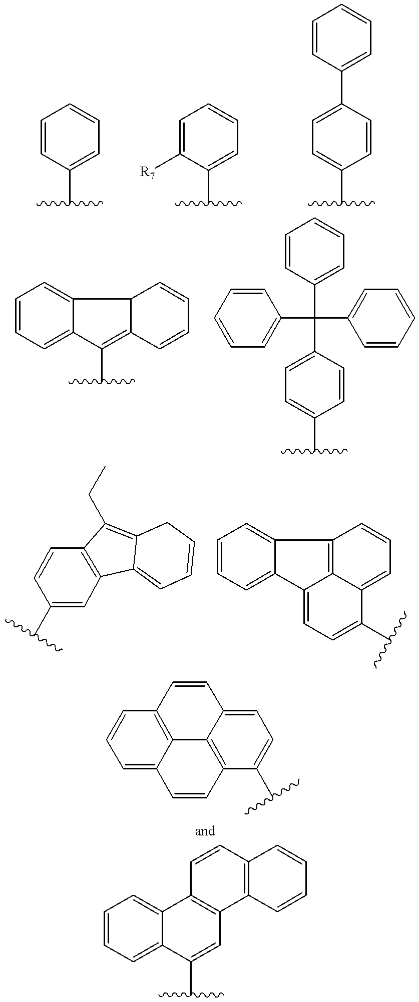
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

111. The device of claim 91, wherein R₃, R_{4,} R₅ and R₆ are each independently selected from the group consisting of: 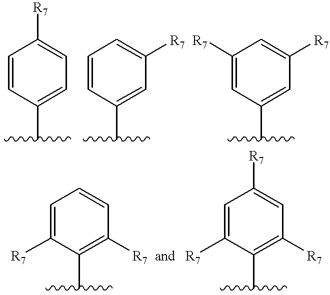
wherein each R₇ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

112. The device of claim 91, wherein R₃, R₄, R₅ and R₆ are each independently selected from the group consisting of: 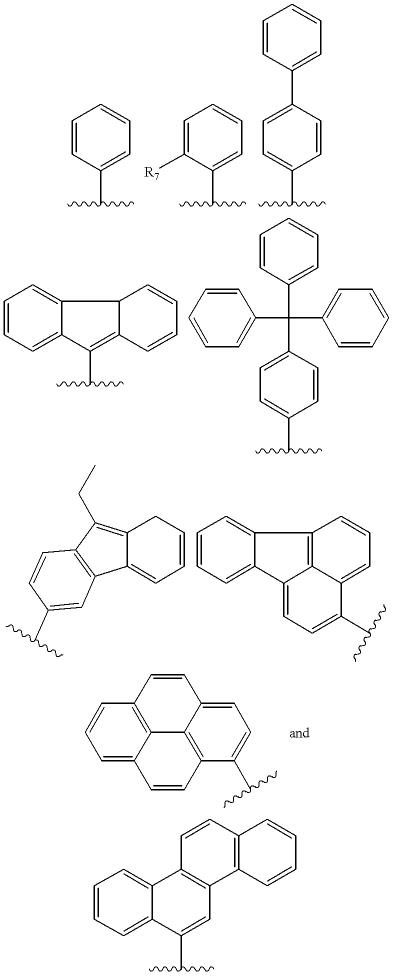
wherein R₇ is selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

113. The compound of claim 1, wherein R₃ and R₄ taken together with the nitrogen atom to which they are attached or R₅ and R₆ taken together with the nitrogen atom to which they are attached are independently selected from the group consisting of:

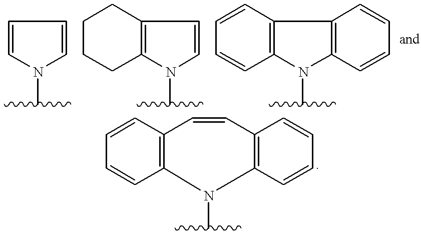

114. The compound of claim 5, wherein R₅ and R₆ taken together with the nitrogen atom to which they are attached are independently selected from the group consisting of:

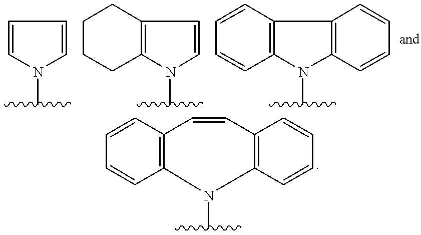

115. The compound of claim 9, wherein R₃ and R₄ taken together with the nitrogen atom to which they are attached are independently selected from the group consisting of:

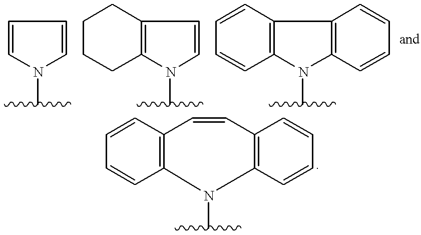

116. The compound of claim 13, wherein R₃ and R₄ taken together with the nitrogen atom to which they are attached or R₅ and R₆ taken together with the nitrogen atom to which they are attached are independently selected from the group consisting of:

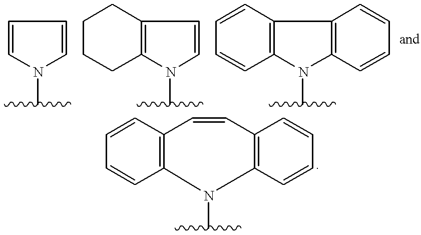

117. The device of claim 16, wherein R₃ and R₄ taken together with the nitrogen atom to which they are attached or R₅ and R₆ taken together with the nitrogen atom to which they are attached are independently selected from the group consisting of:

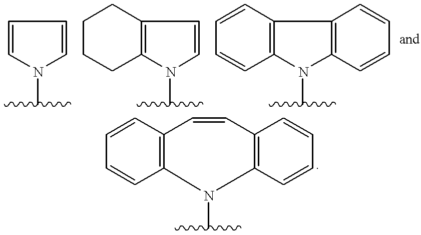

118. The device of claim 47, wherein R₃ and R₄ taken together with the nitrogen atom to which they are attached or R₅ and R₆ taken together with the nitrogen atom to which they are attached are independently selected from the group consisting of:

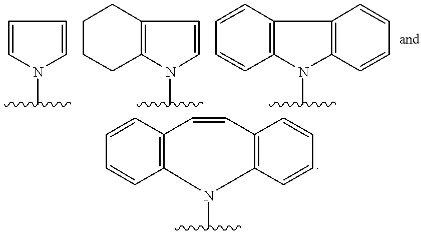

119. The device of claim 80, wherein R₃ and R₄ taken together with the nitrogen atom to which they are attached or R₅ and R₆ taken together with the nitrogen atom to which they are attached are independently selected from the group consisting of:

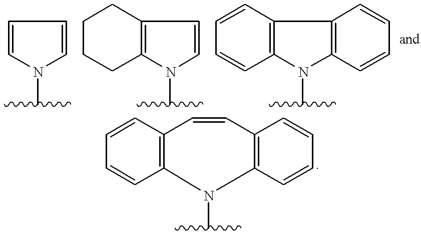

120. The device of claim 90, wherein R₃ and R₄ taken together with the nitrogen atom to which they are attached are independently selected from the group consisting of:

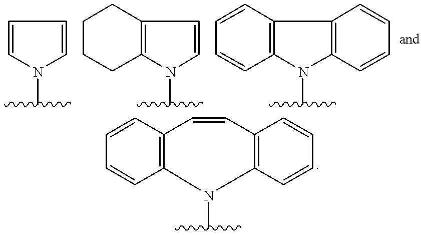

121. The device of claim 91, wherein R₃ and R₄ taken together with the nitrogen atom to which they are attached or R₅ and R₆ taken together with the nitrogen atom to which they are attached are independently selected from the group consisting of:

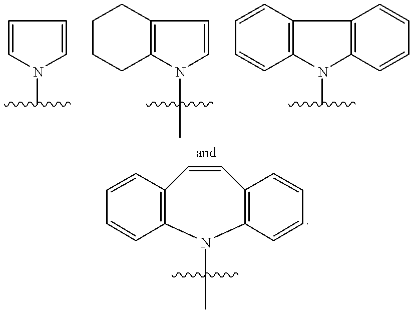

122. A hole-injection or hole-transport compound of formula 1: 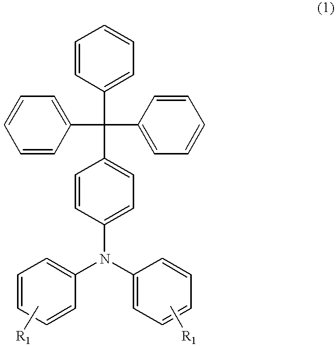
wherein each R₁ is independently selected from the group consisting of hydrogen, C₁-C₆ straight or branched chain alkyl, alkoxy, —CN, —Cl, —F, —CF₃, —SR, —SiR, vinyl that is unsubstituted or substituted at terminal carbon position(s) with C₁-C₆ alkyl or an aromatic group, aryl that is unsubstituted or substituted at para-, meta- or ortho- positions with a C₁-C₆ alkyl, C₁-C₈ alkoxy or —SR, 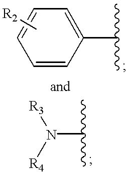
R₂ is selected from the group consisting of C₁-C₆ straight or branched chain alkyl, alkoxy, —CN, —Cl, —F, —CF₃, —SR, —SiR, aryl that is unsubstituted or substituted at para-, meta- or ortho positions with a C₁-C₆ alkyl, C₁-C₈ alkoxy or —SR, and 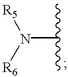
R₃, R_{4,} R₅ and R₆ are each independently selected from the group consisting of: 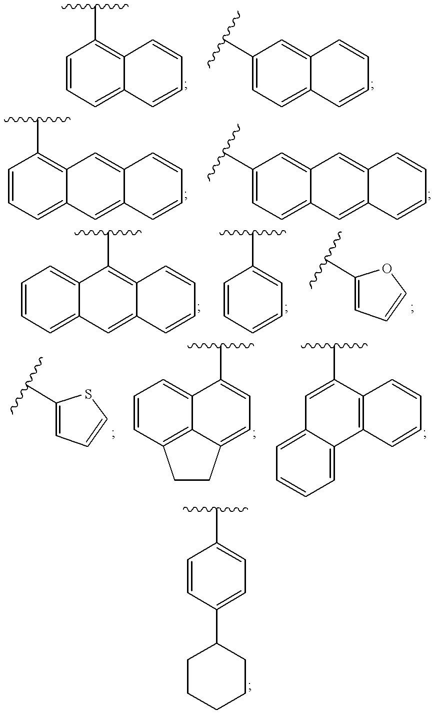
or R₃ and R₄ taken together with the nitrogen atom to which they are attached or R₅ and R₆ taken together with the nitrogen atom to which they are attached are independently selected from the group consisting of: 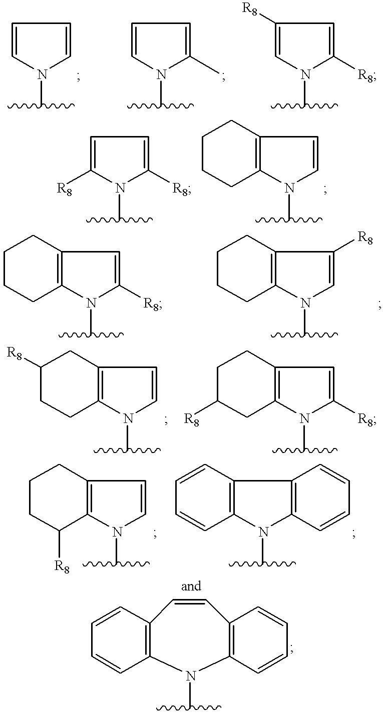
each R₈ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

123. A hole-injection or hole-transport compound of formula 2: 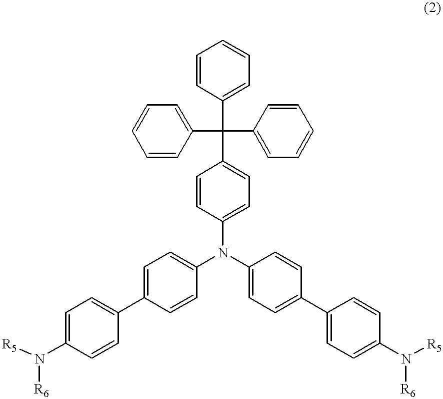
wherein R₅ and R₆ are each independently selected from the group consisting of 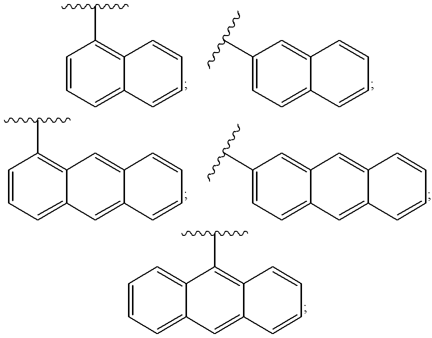 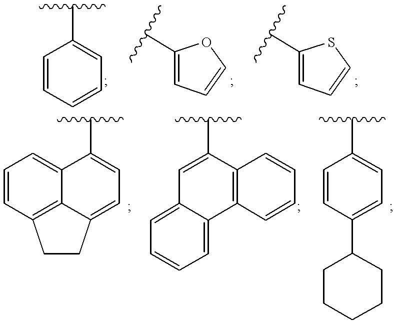
or R₅ and R₆ taken together with the nitrogen atom to which they are attached are independently selected from the group consisting of: 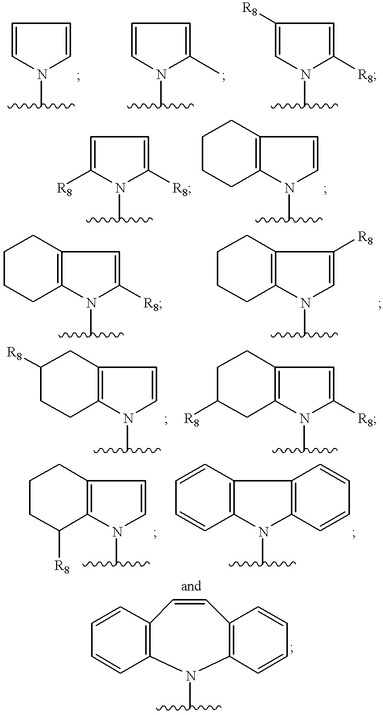
each R₈ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

124. A hole-injection or hole-transport compound of formula 3: 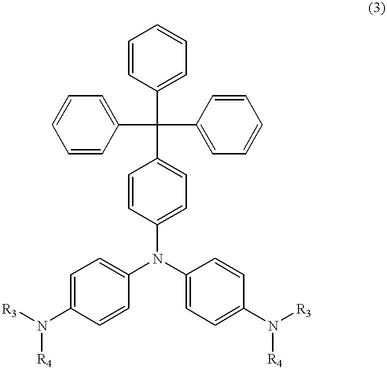
wherein R₃ and R₄ are each independently selected from the group consisting of: 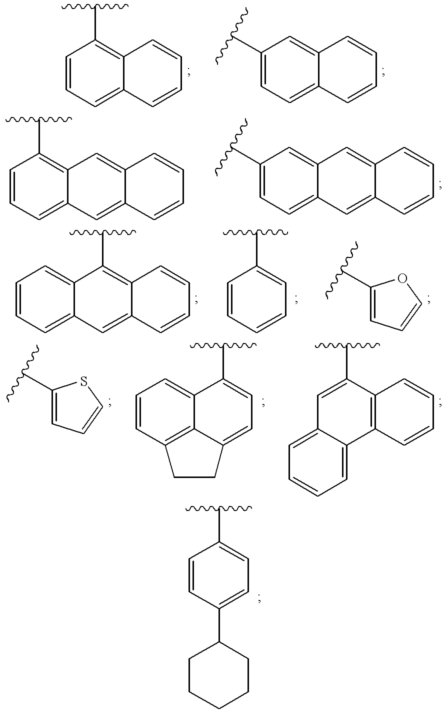
or R₃ and R₄ taken together with the nitrogen atom to which they are attached are independently selected from the group consisting of: 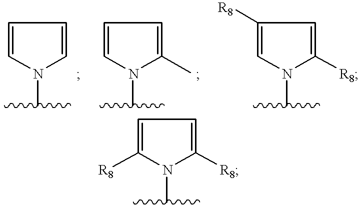 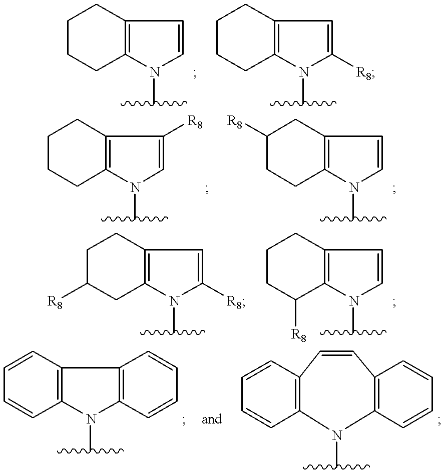
each R₈ is independently selected from the group consisting of —OR₉, C₁-C₄ alkyl, aryl, —SCH₃, —CF₃, —Cl, —Br, —NO₂, —SR, —SiR and —COOR₉;
R is C₁-C₆ straight or branched chain alkyl; and
R₉ is selected from the group consisting of C₁-C₆ alkyl and aryl.

125. A hole-injection or hole-transport compound of formula 4: 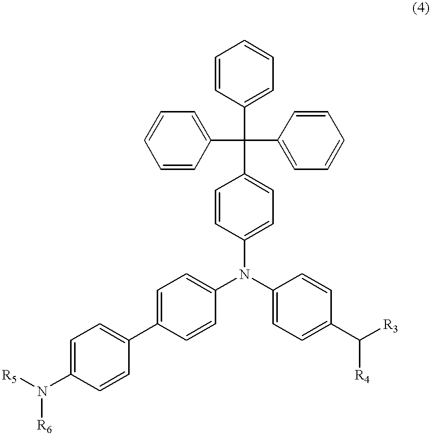
wherein R₃, R_{4,} R₅ and R₆ are each independently selected from the group consisting of 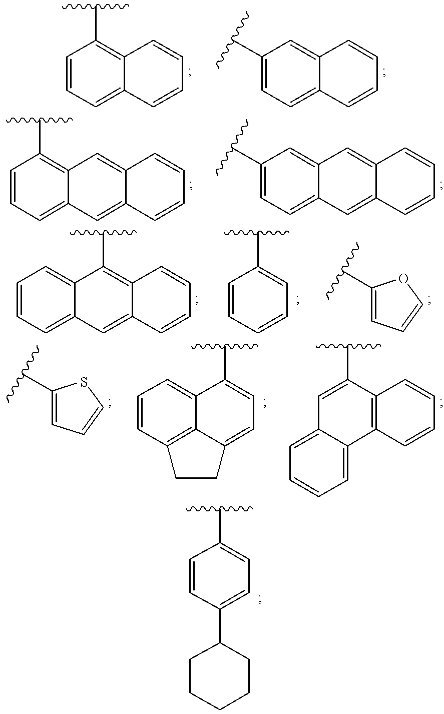
or R₃ and R₄ taken together with the nitrogen to which they are attached or R₅ and R₆ taken together with the nitrogen atom to which they are attached are selected from the group consisting of: 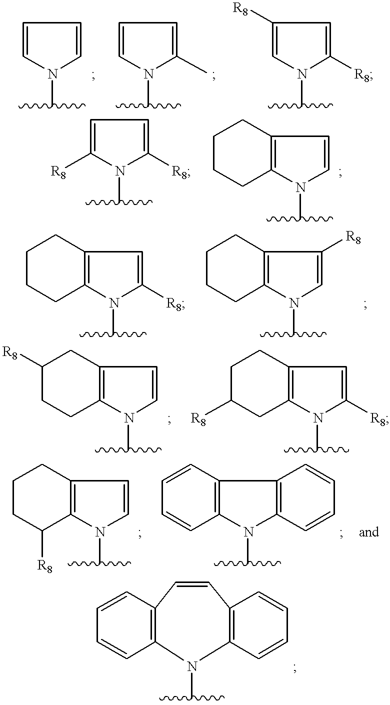
each R₉ is independently selected from the group consisting of —OR₉, C₁-C₄ alkyl, aryl, —SCH₃ , —CF₃ , —Cl, —Br, —NO₂, —SR, —SiR and —COOR₉;
R is C₁-C₆ straight or branched chain alkyl; and
R₉ is selected from the group consisting of C₁-C₆ alkyl and aryl.
